(19) United States
(12) Patent Application Publication
(10) Pub. No.: US 2025/0314671 A1
(43) Pub. Date: Oct. 9, 2025

(54) Diagnostic signature

(71) Applicant: BCAL Diagnostics Limited, Sydney, New South Wales (AU)

(72) Inventors: Amani BATARSEH, Sydney, New South Wales (AU); Dana PASCOVICI, Sydney, New South Wales (AU); Aruna BANSAL, Sydney, New South Wales (AU)

(73) Assignee: BCAL Diagnostics Limited, Sydney, New South Wales (AU)

(21) Appl. No.: 18/864,129

(22) PCT Filed: May 10, 2023
(86) PCT No.: PCT/AU2023/050389

§ 371 Date: Nov. 8, 2024

(30) Foreign Application Priority Data
May 10, 2022 (AU) 2022901245
Oct. 20, 2022 (AU) 2022903097

(51) Int. Cl. G01N 33/92

(52) U.S. Cl. CPC G01N 33/92; G01N 2405/02; G01N 2405/06; G01N 2405/10; G01N 2570/00; G01N 2800/7028

(57) ABSTRACT

The present disclosure relates to methods of diagnosing and treating breast cancer in a subject that include determining a level of one or more lipid biomarkers in a biological sample obtained from the subject. Systems and kits for use in such methods are also provided.

### CROSS-REFERENCE TO RELATED APPLICATIONS

The present application claims priority from Australian Provisional Patent Application No. 2022901245 filed on 10 May 2022 and Australian Provisional Patent Application No. 2022903097 filed on 20 Oct. 2022, the contents of which are incorporated herein by reference in their entirety.

### TECHNICAL FIELD

The present disclosure relates to methods of diagnosing and treating breast cancer.

### BACKGROUND

In 2020, 2.3 million women were diagnosed with breast cancer and there were 685 000 deaths globally. At the end of 2020, there were 7.8 million women alive who had been diagnosed with breast cancer in the preceding 5 years, making it the world's most prevalent cancer (World Health Organisation). Although breast cancer is mostly a disease of females, 1 in 1100 males may also develop the disease (Society, 2016).

The key to surviving breast cancer is early detection and treatment. The current gold standard for detection is via mammogram, however, it is known to be less effective at younger ages.

Accordingly, there remains a need for a more accurate screening test for breast cancer for women of all ages, such as to detect the cancer at a cellular level and before metastasis (Mistry and French, 2016).

### SUMMARY

The present disclosure is based on the surprising discovery of a number of lipid biomarkers, which can readily be detected in order to diagnose breast cancer in women. By extension, these lipid biomarkers (or lipidomic signatures) demonstrate promise in identifying patients that require treatment for breast cancer.

In a first aspect, the present disclosure provides a method of diagnosing a subject with a breast cancer, said method including the step of measuring a level of one or more lipid biomarkers in a biological sample from the subject, wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof, and wherein the level of the one or more lipid biomarkers is diagnostic or indicative of the subject having the breast cancer.

In some examples of the present method, an increased level of Cer(d36:1), Cer(d42:0), DG(34:2), Hex2Cer(d34:1), Hex2Cer(d42:2), LPE(22:6), LPI(20:4), PC(32:1), PC(34:1), PC(35:4), PC(36:2), PC(36:3), PC(37:4), PC(38:5), PE(34:1), PI(32:1), PI(34:1), PS(38:4), PS(40:6), SM(d32:2), SM(d33:1), SM(d35:1), SM(d36:1), SM(d36:2), SM(d38:4), SM(d40:3), SM(d41:2), SM(d41:3), SM(d42:1), SM(d42:4) and/or SM(d44:4), or a fragment, variant or derivative thereof, and/or a decreased level of Cer(42:1), LPC(14:0), LPC(16:0e), LPC(17:1), LPC(18:3), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and/or TG(62:2), or a fragment, variant or derivative thereof, is diagnostic or indicative of the subject having the breast cancer.

Suitably, the present method further includes the step of administering a treatment for the breast cancer to the subject.

In a second aspect, the present disclosure provides a method for measuring a level of one or more lipid biomarkers in a biological sample from a subject, said method including the steps of:
(a) providing the biological sample; and
(b) measuring the level of the one or more lipid biomarkers in the biological sample.

wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1). LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

Suitably, the subject is suspected of having a breast cancer or has been previously diagnosed with a breast cancer.

In some examples, the measuring step includes determining the presence or absence of: (i) an increased level of Cer(d36:1), Cer(d42:0), DG(34:2), Hex2Cer(d34:1), Hex2Cer(d42:2), LPE(22:6), LPI(20:4), PC(32:1), PC(34:1), PC(35:4), PC(36:2), PC(36:3), PC(37:4), PC(38:5), PE(34:1), PI(32:1), PI(34:1), PS(38:4), PS(40:6), SM(d32:2), SM(d33:1), SM(d35:1), SM(d36:1), SM(d36:2), SM(d38:4), SM(d40:3), SM(d41:2), SM(d41:3), SM(d42:1), SM(d42:4) and/or SM(d44:4), or a fragment, variant or derivative thereof, and/or (ii) a decreased level of Cer(42:1), LPC(14:0), LPC(16:0e), LPC(17:1), LPC(18:3), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and/or TG(62:2), or a fragment, variant or derivative thereof, in the biological sample of the subject.

In a third aspect, the present disclosure provides a method of treating a breast cancer in a subject, said method including the step of performing a treatment in respect of the subject in which a level of one or more lipid biomarkers has been measured in a biological sample therefrom that is diagnostic or indicative of the subject having the breast cancer, wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

Suitably, the treatment includes administering a therapeutically effective amount of an anti-cancer treatment to the subject.

In some examples of the present method, an increased level of Cer(d36:1), Cer(d42:0), DG(34:2), Hex2Cer(d34:1), Hex2Cer(d42:2), LPE(22:6), LPI(20:4), PC(32:1), PC(34:1), PC(35:4), PC(36:2), PC(36:3), PC(37:4), PC(38:5), PE(34:1), PI(32:1), PI(34:1), PS(38:4), PS(40:6), SM(d32:2), SM(d33:1), SM(d35:1), SM(d36:1), SM(d36:2), SM(d38:4), SM(d40:3), SM(d41:2), SM(d41:3), SM(d42:1), SM(d42:4) and/or SM(d44:4), or a fragment, variant or derivative thereof, and/or a decreased level of Cer(42:1), LPC(14:0), LPC(16:0e), LPC(17:1), LPC(18:3), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and/or TG(62:2), or a fragment, variant or derivative thereof, was measured from the biological sample of the subject.

Referring to the methods of the aforementioned aspects, the one or more lipid biomarkers suitably comprise LPC(14:0), or a fragment, variant or derivative thereof, and one or more other lipid biomarkers selected from the group consisting of Cer(d36:1), Cer(d42:0), Cer(d42:1), DG(34:2), Hex2Cer(d34:1), Hex2Cer(d42:2), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

For the above aspects, the one or more lipid biomarkers suitably comprise PI(38:6), or a fragment, variant or derivative thereof, and one or more other lipid biomarkers selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3). SM(d42:1), SM(d42:4), SM(d44:4), TG(52:3e), TG(53:4), TG(54:3), TG(54:4). TG(54:5). TG(54:6), TG(56:1), TG(57:1), TG(58:1). TG(58:2), TG(58:3), TG(59:2), TG(60:1) and TG(62:2), or a fragment, variant or derivative thereof.

In some examples of the methods of the aforementioned aspects, the one or more lipid biomarkers comprise LPC(14:0) and PI(38:6), or a fragment, variant or derivative thereof, and optionally one or more other lipid biomarkers selected from the group consisting of Cer(d36:1), Cer(d42:0), Cer(d42:1), DG(34:2), Hex2Cer(d34:1), Hex2Cer(d42:2), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

In certain examples of the methods of the above aspects, the one or more lipid biomarkers are selected from the group consisting of LPC(14:0), PC(32:1), PC(38:5), PE(34:1), PS(38:4), SM(d36:2), SM(d38:4), TG(54:4), TG(56:1) and TG(58:2), or a fragment, variant or derivative thereof.

In other examples of the methods of the above aspects, the one or more lipid biomarkers are selected from the group consisting of LPC(14:0), PC(32:1), PC(36:2), PC(38:5), PE(34:1), PI(34:1), PS(38:4), SM(d36:2), SM(d38:4), SM(d44:4), TG(52:3e), TG(53:4), TG(54:4), TG(54:5), TG(54:6), TG(56:1), TG(58:2) and TG(58:3), or a fragment, variant or derivative thereof.

In various examples of the methods of the above aspects, the one or more lipid biomarkers are selected from the group consisting of LPC(14:0), PE(34:1), PS(38:4), SM(d36:2), TG(52:3e) and TG(58:3), or a fragment, variant or derivative thereof.

In certain examples of the methods of the above aspects, the one or more lipid biomarkers are selected from the group consisting of LPC(14:0), LPC(16:0e), LPE(22:6), PE(34:2p), PE(38:3p), PI(36:1), PI(38:6), PS(38:4), PS(40:6), SM(d33:1), SM(d35:1) and TG(57:1), or a fragment, variant or derivative thereof. To this end, the one or more lipid biomarkers may comprise:
(a) LPC(14:0), PS(38:4) and TG(57:1), or a fragment, variant or derivative thereof:
(b) LPC(16:0e), LPE(22:6) and PI(38:6), or a fragment, variant or derivative thereof;
(c) PE(34:2p), PE(38:3p), PI(36:1), PI(38:6) and PS(40:6), or a fragment, variant or derivative thereof;
(d) LPC(14:0), PI(38:6), and SM(d33:1), or a fragment, variant or derivative thereof; or
(e) LPC(14:0), PI(38:6), and SM(d35:1), or a fragment, variant or derivative thereof;
and optionally one or more other lipid biomarkers selected from the group consisting of LPC(14:0), LPC(16:0e), LPE(22:6), PE(34:2p), PE(38:3p), PI(36:1), PI(38:6), PS(38:4), PS(40:6), SM(d33:1), SM(d35:1) and TG(57:1), or a fragment, variant or derivative thereof.

In various examples of the methods of the above aspects, the one or more lipid biomarkers described herein are selected from the group consisting of LPC(14:0), LPE(22:6), PI(38:6), PE(34:2p), SM(d35:1), PS(38:4) and PS(38:4), or a fragment, variant or derivative thereof.

Suitably, for the above aspects, the level of the one or more lipid biomarkers is or has been measured, at least in part, by mass spectrometry.

Suitably, the predictive accuracy of the methods of the aforementioned aspects, as determined by an ROC AUC value, is at least about 0.65, at least about 0.70, at least about 0.75 or at least about 0.80.

In a fourth aspect, the present disclosure provides a system for determining the presence or absence of a breast cancer in a subject, the system comprising:
a mass spectrometry unit configured for determining a level of one or more lipid biomarkers in a biological sample obtained from the subject, wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof; and
a processing unit configured for using or analysing the level of the one or more lipid biomarkers to determine the presence or absence of the breast cancer in the subject.

In a fifth aspect, the present disclosure provides a kit for determining the presence or absence of a breast cancer in a subject, the kit comprising one or more reagents for determining a level of one or more lipid biomarkers in a biological sample obtained from the subject, wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

Suitably, the one or more reagents comprise one or more probes, each probe being specific or selective for one of the one or more lipid biomarkers.

Referring to the above aspects, the one or more lipid biomarkers suitably comprises two or more lipid biomarkers, three or more lipid biomarkers, four or more lipid biomarkers or five or more lipid biomarkers selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

In some examples of the above aspects, the one or more lipid biomarkers comprises two or more lipid biomarkers, three or more lipid biomarkers, four or more lipid biomarkers or five or more lipid biomarkers selected from the group consisting of LPC(14:0), PC(32:1), PC(38:5), PE(34:1), PS(38:4), SM(d36:2), SM(d38:4), TG(54:4), TG(56:1) and TG(58:2), or a fragment, variant or derivative thereof.

In other examples of the above aspects, the one or more lipid biomarkers comprises two or more lipid biomarkers, three or more lipid biomarkers, four or more lipid biomarkers or five or more lipid biomarkers selected from the group consisting of LPC(14:0), PC(32:1), PC(36:2), PC(38:5), PE(34:1), PI(34:1), PS(38:4), SM(d36:2), SM(d38:4), SM(d44:4), TG(52:3e), TG(53:4), TG(54:4), TG(54:5), TG(54:6), TG(56:1), TG(58:2) and TG(58:3), or a fragment, variant or derivative thereof.

In particular examples of the above aspects, the one or more lipid biomarkers comprises two or more lipid biomarkers, three or more lipid biomarkers, four or more lipid biomarkers or five or more lipid biomarkers selected from the group consisting of LPC(14:0), PE(34:1), PS(38:4), SM(d36:2), TG(52:3e) and TG(58:3), or a fragment, variant or derivative thereof.

In various examples of the above aspects, the one or more lipid biomarkers comprises two or more lipid biomarkers, three or more lipid biomarkers, four or more lipid biomarkers or five or more lipid biomarkers selected from the group consisting of LPC(14:0), LPC(16:0e), LPE(22:6), PE(34:2p), PE(38:3p), PI(36:1), PI(38:6), PS(38:4), PS(40:6), SM(d33:1), SM(d35:1) and TG(57:1), or a fragment, variant or derivative thereof.

In regards to the aforementioned aspects, the biological sample suitably is or comprises a blood sample, a plasma sample and/or a serum sample.

Suitably, the system of the fourth aspect or the kit of the fifth aspect are for use in the method of the first, second or third aspects.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein. It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

FIG. 1. Violin Plots of lipids with feature importance >0.05 for (a) LPC(14:0); (b) TG(16:0_18:1_23:0); (c) LPC(18:3); and (d) PS(18:0_20:4).

FIG. 2. Violin Plots of lipids with feature importance >0.05 for (a) PI(18:2_20:4); (b) LPE(22:6); (c) LPC(16:0e): (d) Cer(d18:1_24:0); and (e) PE(16:0_20:4).

FIG. 3. Violin Plots of lipids with feature importance >0.05 for (a) PI(18:2_20:4); (b) PE(16:0p_18:2); (c) PI(18:0_18:1); (d) PS(18:0_22:6); and (e) PE(20:1p_18:2).

FIG. 4. Violin Plots of lipids with feature importance >0.05 for: (a) LPC(14:0); (b) SM(d18:1_15:0); (c) LPC(17:1); (d) PI(18:2_20:4); and (e) SM(d41:2).

FIG. 5. Violin Plots of lipids with feature importance >0.05 for: (a) PI(18:2_20:4); (b) LPC(14:0); and (c) SM(d35:1).

FIG. 6. Overlaid ROC for candidate signature arising from dataset 1

FIG. 7. Overlaid ROC for candidate signature arising from dataset 2

FIG. 8. Overlaid ROC for candidate signature arising from dataset 3

FIG. 9. Overlaid ROC for candidate signature arising from dataset 4

FIG. 10. Overlaid ROC for candidate signature arising from dataset 234 (Validation)

FIG. 11. Volcano plot showing fold change and log p-value-more significant changes are at the top. The initial set of markers was selected based on significance value (p-value <0.05) after an initial filtering: lipid ions shown since full identifier not available in earlier stages.

FIG. 12. Filtering based on fold change and p-values was used to select an additional set of 18 markers from Cohort 3, and the combined sets were then condensed using stepwise regression and significance in logistic models.

FIG. 13. In this approach Cohort 4 was used predominantly for validation as set out in Example 1. Repeating the process of identifying differentially expressed lipids for Cohort 4 shows four of the biomarkers identified in Example 1 were present in the top 10 selections; these were included in a small panel of 6 common markers to both approaches, “Common 6-ML_GBM”.

FIG. 14. Initial panel of 18 established using Cohort 1 and 2-performance estimated on next available cohort (Cohort3) via leave one out cross-validation.

FIG. 15. Second panel of 18 established using Cohorts 1-3-performance estimated on next available cohort (Cohort4) via leave one out cross-validation.

FIG. 16. Panel of six-lipid 1 boxplot (order of each dataset pair from left to right: control, breast cancer)

FIG. 17. Panel of six-lipid 2 boxplot (order of each dataset pair from left to right: control, breast cancer)

FIG. 18. Panel of six-lipid 2 boxplot, same m/z (order of each dataset pair from left to right: control, breast cancer)

FIG. 19. Panel of six-lipid 3 boxplot (order of each dataset pair from left to right: control, breast cancer)

FIG. 20. Panel of six-lipid 3 boxplot; same m/z (order of each dataset pair from left to right: control, breast cancer)

FIG. 21. Panel of six-lipid 4 boxplot (order of each dataset pair from left to right: control, breast cancer)

FIG. 22. Panel of six-lipid 5 boxplot (order of each dataset pair from left to right: control, breast cancer)

FIG. 23. Panel of six-lipid 6 boxplot (order of each dataset pair from left to right: control, breast cancer)

FIG. 24. ROC curve performance of panel of 18 markers estimated on Cohort 2 via leave one out cross-validation.

FIG. 25. ROC curve performance of panel of 18 markers estimated on Cohort 3 via leave one out cross-validation.

FIG. 26. ROC curve performance of panel 400 reoptimised estimated on Cohort 4 via leave one out cross-validation.

FIG. 27. ROC curve performance of panel 400 reoptimised estimated on Cohorts 3 and 4 via leave one out cross-validation.

FIG. 28. ROC curve performance of restricted 9 lipid panel estimated on Cohort 2 via leave one out cross-validation.

FIG. 29. ROC curve performance of panel of top 10 optimised estimated on Cohorts 2 and 3 via leave one out cross-validation.

FIG. 30. ROC curve performance of panel of 6 four markers estimated on Cohorts 2 to 4 via leave one out cross-validation.

FIG. 31. ROC curve performance of panel of 6 four markers estimated on Cohorts 3 and 4 via leave one out cross-validation.

FIG. 32. ROC curve performance of panel of common 6 ML-GBM estimated on Cohorts 3 and 4 via leave one out cross-validation.

FIG. 33. ROC curve performance of panel of Cohort2Optimised estimated on Cohort 2 via leave one out cross-validation.

FIG. 34. The lipids from the three BCAL panels are overlaid on the volcano plots (fold change is fasted/fed). Some of the markers are differentially expressed between fed and fasted states. None of the ML-GBM panel lipids (red) are differentially expressed (i.e. present at significantly different concentrations) between fasted/fed.

FIG. 35. Correlation of ML-GBM signature scores obtained on various combinations of sample types.

FIG. 36. ML-GBM scores boxplots showing median and inter-quartile range for all sample categories: NEV=non-fasted EV, FEV=fasted EV, FP=fasted plasma, NV=non-fasted plasma. Low scores represent a correct “control” classification.

### DETAILED DESCRIPTION

### General Techniques and Definitions

Unless specifically defined otherwise, all technical and scientific terms used herein shall be taken to have the same meaning as commonly understood by one of ordinary skill in the art (e.g. in genomics, immunology, molecular biology, immunohistochemistry, biochemistry, oncology, and pharmacology).

The present disclosure is performed using, unless otherwise indicated, conventional techniques of molecular biology, microbiology, recombinant DNA technology and immunology. Such procedures are described, for example in Sambrook, Fritsch & Maniatis, Molecular Cloning: A Laboratory Manual, Cold Spring Harbor Laboratories, New York, Fourth Edition (2012), whole of Vols I, II, and III: DNA Cloning: A Practical Approach, Vols. I and II (D. N. Glover, Second Edition., 1995), IRL Press, Oxford, whole of text: Oligonucleotide Synthesis: A Practical Approach (M. J. Gait, ed, 1984) IRL Press, Oxford, whole of text, and particularly the papers therein by Gait, ppl-22; Atkinson et al, pp 35-81; Sproat et al, pp 83-115; and Wu et al, pp 135-151:4. Nucleic Acid Hybridization: A Practical Approach (B. D. Hames & S. J. Higgins, eds., 1985) IRL Press, Oxford, whole of text: Immobilized Cells and Enzymes: A Practical Approach (1986) IRL Press, Oxford, whole of text: Perbal, B., A Practical Guide to Molecular Cloning (1984) and Methods In Enzymology (S. Colowick and N. Kaplan, eds., Academic Press, Inc.), whole of series.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment of the present disclosure may be applied mutatis mutandis to any other aspect or embodiment of the present disclosure.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, the singular forms of “a”, “and” and “the” include plural forms of these words, unless the context clearly dictates otherwise.

The term “and/or”, e.g., “X and/or Y” shall be understood to mean either “X and Y” or “X or Y” and shall be taken to provide explicit support for both meanings or for either meaning.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### Methods of Diagnosis

The inventors have surprisingly shown that the concentration level of particular lipids from a lipidomic signature in biological samples, such as blood samples, can be used to diagnose subjects as having breast cancer. Advantageously, such a method may allow a physician to make appropriate, informed, and timely follow-up and treatment decisions based on this information.

Accordingly, the inventors have developed methods of diagnosing breast cancer.

As such, in one broad form, the present disclosure provides a method for measuring a level of one or more lipid biomarkers in a biological sample from a subject, said method including the steps of:
(a) providing the biological sample; and
(b) measuring the level of the one or more lipid biomarkers in the biological sample, wherein the one or more lipid biomarkers are selected from the group consisting of Cer(d36:1), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

In a related broad form, the present disclosure provides a method of diagnosing a subject with a breast cancer, said method including the step of measuring a level of one or more lipid biomarkers in a biological sample from the subject, wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof, and wherein the level of the one or more lipid biomarkers is diagnostic or indicative of the subject having the breast cancer.

With respect to the aspects described herein, the term “subject” includes, but is not limited to, mammals, inclusive of humans, performance animals (such as horses, camels, greyhounds), livestock (such as cows, sheep, horses) and companion animals (such as cats and dogs). In one example, the subject is a human. In certain examples, the subject is a female human. In other examples, the subject is a male human.

As generally used herein, the terms “cancer”, “tumour”, “malignant” and “malignancy” refer to diseases or conditions, or to cells or tissues associated with the diseases or conditions, characterized by aberrant or abnormal cell proliferation, differentiation and/or migration often accompanied by an aberrant or abnormal molecular phenotype that includes one or more genetic mutations or other genetic changes associated with oncogenesis, expression of tumour markers, loss of tumour suppressor expression or activity and/or aberrant or abnormal cell surface marker expression.

The term “breast cancer” refers to a condition characterized by abnormally rapid growth of abnormal cells in one or both breasts of a subject. Breast cancer can include, but is not limited to, ductal carcinoma in situ (DCIS), invasive breast cancer (e.g., an invasive carcinoma), inflammatory breast cancer, angiosarcoma of the breast, Phyllodes tumours of the breast, and/or Paget's disease of the nipple. As used herein, “invasive carcinoma” or “invasive breast cancer” refers to a type of cancer that can include, but is not limited to, invasive ductal carcinoma (IDC), infiltrating ductal carcinoma, invasive lobular carcinoma (ILC), adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, micropapillary carcinoma, and/or mixed carcinoma having features of both invasive ductal and lobular. Suitably, the breast cancer to be diagnosed in a subject is selected from IDC, DCIS and ILC. In particular examples, the breast cancer to be diagnosed in a subject is IDC. In certain examples, the breast cancer to be diagnosed in a subject is DCIS. In other examples, the breast cancer to be diagnosed in a subject is ILC.

The skilled person will further appreciate that the breast cancer may include any aggressive breast cancers and cancer subtypes known in the art, such as triple negative breast cancer, lymph node positive (LN+) breast cancer, HER2 positive (HER2+) breast cancer, PR negative (PR⁻) breast cancer, PR positive (PR) breast cancer, ER negative (ER⁻) breast cancer and ER positive (ER+) breast cancer.

The breast cancer also may be of any stage or grade (e.g., Stages I, II, III or IV) and as such can include metastatic breast cancer. Suitably, the breast cancer to be diagnosed is an early stage cancer (e.g., Stage 1 or Stage 2 breast cancer). In particular examples, the breast cancer to be diagnosed is Stage 1 breast cancer. In other examples, the breast cancer to be diagnosed is Stage 2 breast cancer. Alternatively, the breast cancer to be diagnosed can be a late stage cancer (e.g., Stage 3 or Stage 4 breast cancer). In particular examples, the breast cancer to be diagnosed is Stage 3 breast cancer. In other examples, the breast cancer to be diagnosed is Stage 4 breast cancer.

As used herein, the terms “diagnosis” and “diagnosing” refers to a method by which one of ordinary skill in the art can assess and/or determine whether a patient or subject is suffering from a given disease or condition, such as determining the presence or absence of a breast cancer. Those skilled in the art often make a diagnosis based on one or more diagnostic indicators or markers whose presence, absence, or amount indicates the presence or absence of the disease, disorder or condition. It will further be appreciated that these terms do not indicate the ability to determine the presence or absence of a particular disease with 100% accuracy, nor do they indicate that a given course or outcome is more likely to occur. Rather, one of ordinary skill in the art will understand that the terms “diagnosis” and “diagnosing” refer to an increased probability that a subject will have a certain disease, disorder or condition, such as a breast cancer.

Suitably, the methods described herein are performed in conjunction (e.g., before and/or after) with one or more further diagnostic tests as are known in the art (e.g., breast exam: breast imaging, such as mammogram, ultrasound and MRI: biopsy). In this regard, the present method may be utilised as a preliminary screening test to identify subjects who may benefit from further diagnostic testing. Additionally, or alternatively, the present method may be utilised to confirm the presence or absence of breast cancer as indicated by a previous diagnostic test. Accordingly, in some examples, the present method may include the initial or earlier step and/or subsequent step of performing one or more further diagnostic tests on the subject in question. In alternative examples, the methods described herein are performed without any further diagnostic testing as a primary diagnostic test for breast cancer.

Suitably, if the level, such as a concentration level or an expression level, of the one or more lipid biomarkers is altered or modulated in the biological sample from a subject, this can be diagnostic of breast cancer in the subject. In one example, an increased level of expression or concentration of a first subset of the one or more lipid biomarkers and/or a decreased level of expression or concentration of a second subset of the one or more lipid biomarkers is diagnostic or indicative of the subject having the breast cancer. As demonstrated in the Examples herein, certain lipid biomarkers (e.g., PE(34:1), PI(32:1), PI(34:1), PS(38:4) and PS(40:6)) may demonstrate an increased expression or concentration level in a first cohort of subjects that is diagnostic of breast cancer, whilst demonstrating a decreased expression or concentration level in a second cohort of subjects that is also diagnostic of breast cancer. Accordingly, a decreased or an increased level of such lipid biomarkers, such as relative to a threshold or reference level, may be indicative or diagnostic of breast cancer in a subject.

In particular examples, an increased level of Cer(d36:1), Cer(d42:0), DG(34:2), Hex2Cer(d34:1), Hex2Cer(d42:2), LPE(22:6), LPI(20:4), PC(32:1), PC(34:1), PC(35:4), PC(36:2), PC(36:3), PC(37:4), PC(38:5), PE(34:1), PI(32:1), PI(34:1), PS(38:4), PS(40:6), SM(d32:2), SM(d33:1), SM(d35:1), SM(d36:1), SM(d36:2), SM(d38:4), SM(d40:3), SM(d41:2), SM(d41:3), SM(d42:1), SM(d42:4) and/or SM(d44:4), or a fragment, variant or derivative thereof, and/or a decreased level of Cer(42:1), LPC(14:0), LPC(16:0e), LPC(17:1), LPC(18:3), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and/or TG(62:2), or a fragment, variant or derivative thereof, is diagnostic or indicative of the subject having the breast cancer.

In certain examples, the measuring step includes determining the presence or absence of: (i) an increased level of Cer(d36:1), Cer(d42:0), DG(34:2), Hex2Cer(d34:1), Hex2Cer(d42:2), LPE(22:6), LPI(20:4), PC(32:1), PC(34:1), PC(35:4), PC(36:2), PC(36:3), PC(37:4), PC(38:5), PE(34:1), PI(32:1), PI(34:1), PS(38:4), PS(40:6), SM(d32:2), SM(d33:1), SM(d35:1), SM(d36:1), SM(d36:2), SM(d38:4), SM(d40:3), SM(d41:2), SM(d41:3), SM(d42:1), SM(d42:4) and/or SM(d44:4), or a fragment, variant or derivative thereof, and/or (ii) a decreased level of Cer(42:1), LPC(14:0), LPC(16:0e), LPC(17:1), LPC(18:3), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and/or TG(62:2), or a fragment, variant or derivative thereof, in the biological sample of the subject.

According to particular examples, the measuring step includes determining the presence or absence of a decreased level of LPC(14:0) and/or PI(38:6), or a fragment, variant or derivative thereof, in the biological sample of the subject. More particularly, the measuring step may include determining the presence or absence of a decreased level of LPC(14:0), or a fragment, variant or derivative thereof, in the biological sample of the subject. Similarly, the measuring step may include determining the presence or absence of a decreased level of PI(38:6), or a fragment, variant or derivative thereof, in the biological sample of the subject. Even more particularly, the measuring step may include determining the presence or absence of a decreased level of LPC(14:0) and PI(38:6), or a fragment, variant or derivative thereof, in the biological sample of the subject.

Suitably, the present methods further include the step of determining or calculating a risk or diagnostic score from the level of the one or more lipid biomarkers, such as by methods described herein. In some examples, a diagnostic score of 55%, 60%, 65%, 70%, 75%, 80%, 85%, 90%, 95%, 96%, 97%, 98%, 99% or higher is determined for the subject.

Suitably, the diagnostic methods described herein may include the step of administering a treatment to the subject. By way of example, this can include administering to the subject a therapeutically effective amount of the treatment, such as those anti-cancer treatments described herein, when the level of the one or more lipid biomarkers (and/or a risk or diagnostic score derived therefrom) is diagnostic or indicative of the subject having the breast cancer.

### Methods of Treatment

Further to the above, the methods described herein may improve patient outcomes by diagnosing subjects with breast cancer, who could potentially benefit from a treatment thereof.

Accordingly, the inventors have developed methods of treating a breast cancer in a subject.

In one broad form, the present disclosure provides a method of treating a breast cancer in a subject, said method including the step of performing a treatment in respect of the subject, such as surgery and/or administering a therapeutically effective amount of an anti-cancer treatment, in which a level of one or more lipid biomarkers has been measured in a biological sample therefrom that is diagnostic or indicative of the subject having the breast cancer, wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

Suitably, the present method includes the initial step of measuring the level of the one or more lipid biomarkers in the biological sample from the subject.

Suitably, for the present method, a risk or diagnostic score has been determined using the level, such as a concentration level or an expression level, of the one or more lipid biomarkers. The risk or diagnostic score can be diagnostic or indicative of the subject having the breast cancer. In certain examples, the diagnostic score is generated at least in part via a logistic model. To this end, the diagnostic score can be in the form of a probability of the subject having the breast cancer, such that in the absence of additional information a score of 50% or above provides that the subject has a higher probability of having breast cancer than not having breast cancer. In some examples, a diagnostic score of 55%, 60%, 65%, 70%, 75%, 80%, 85%, 90%, 95%, 96%, 97%, 98%, 99% or higher was determined for the subject.

As used herein, the term “therapeutically effective amount” describes a quantity of a specified agent (e.g., an anti-cancer agent) or treatment, such as chemotherapy, radiation therapy, a molecularly targeted therapy and immunotherapy, sufficient to achieve a desired effect in a subject being treated with that agent. For example, this can be the amount of a composition comprising one or more agents that are necessary to reduce, alleviate and/or prevent a cancer or cancer associated disease, disorder or condition. In some examples, a “therapeutically effective amount” is sufficient to reduce or eliminate a symptom of a cancer. In other examples, a “therapeutically effective amount” is an amount sufficient to achieve a desired biological effect, for example an amount that is effective to decrease or prevent cancer growth and/or metastasis.

Ideally, a therapeutically effective amount of an agent is an amount sufficient to induce the desired result without causing a substantial cytotoxic effect in the subject. The effective amount of an agent useful for reducing, alleviating and/or preventing a breast cancer will be dependent on the subject being treated, the type and severity of any associated disease, disorder and/or condition (e.g., the number and location of any associated metastases), and the manner of administration of the therapeutic composition.

Suitably, the various agents, anti-cancer agents or cancer treatments described herein are administered to a subject as a pharmaceutical composition comprising a pharmaceutically-acceptable carrier, diluent or excipient. In this regard, any dosage form and route of administration, such as those provided therein, may be employed for providing a subject with the composition of the present disclosure.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, liposomes and other lipid-based carriers, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991), which is incorporated herein by reference.

Any safe route of administration may be employed for providing a patient with the composition of the present disclosure. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present disclosure suitable for oral or parenteral administration may be presented as discrete units such as capsules, sachets or tablets each containing a pre-determined amount of one or more therapeutic agents of the present disclosure, as a powder or granules or as a solution or a suspension in an aqueous liquid, a non-aqueous liquid, an oil-in-water emulsion or a water-in-oil liquid emulsion. Such compositions may be prepared by any of the methods of pharmacy, which may include the step of bringing into association one or more agents as described above with the carrier which constitutes one or more necessary ingredients. In general, the compositions are prepared by uniformly and intimately admixing the agents of the present disclosure with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present disclosure, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

It is envisaged that the various agents, anti-cancer agents or cancer treatments described herein can be formulated as discrete doses, such as in the form of a kit. Such a kit may further comprise a package insert comprising printed instructions for simultaneous, concurrent, sequential, successive, alternate or separate use of the agents in the treatment, amelioration and/or prevention of cancer, as described herein, in a patient in need thereof. Accordingly, the aforementioned kits are suitably for use in a method of treating, ameliorating and/or preventing breast cancer, inclusive of one or more symptoms, consequences, sequelae or complications thereof, as described herein.

Alternatively, the various therapeutic agents described herein can be formulated together in a composition that optionally includes a pharmaceutically acceptable carrier, excipient or diluent.

Methods of treating breast cancer may be prophylactic, preventative or therapeutic and suitable for the treatment of breast cancer in mammals, particularly humans. As used herein, “treating”, “treat” or “treatment” refers to a therapeutic intervention, course of action or protocol that at least ameliorates a symptom of cancer after the cancer and/or its symptoms have at least started to develop. As used herein, “preventing”, “prevent” or “prevention” refers to therapeutic intervention, course of action or protocol initiated prior to the onset of cancer and/or a symptom of cancer so as to prevent, inhibit or delay or development or progression of the cancer or the symptom.

### Anti-Cancer Treatments

The skilled person will appreciate that cancer treatments for use in the methods described herein may include drug therapy, chemotherapy, antibody, nucleic acid and other biomolecular therapies, radiation therapy, surgery, nutritional therapy, relaxation or meditational therapy and other natural or holistic therapies, although without limitation thereto. Generally, drugs, biomolecules (e.g., antibodies, inhibitory nucleic acids such as siRNA) or chemotherapeutic agents are referred to herein as “anti-cancer therapeutic agents” or “anti-cancer agents”.

Suitably, the treatment is or comprises one or more of surgery (e.g., lumpectomy or mastectomy), chemotherapy, radiation therapy, molecularly targeted therapy and immunotherapy.

As generally used herein, the term “chemotherapy” or “chemotherapeutic agent” broadly refers to a treatment or agent with a cytostatic or cytotoxic agent (i.e., a compound) to reduce or eliminate the growth or proliferation of undesirable cells, such as cancer cells. Accordingly, the terms can refer to a cytotoxic or cytostatic agent used to treat a proliferative disorder, for example cancer. The cytotoxic effect of the agent can be, but is not required to be, the result of one or more of nucleic acid intercalation or binding. DNA or RNA alkylation, inhibition of RNA or DNA synthesis, the inhibition of another nucleic acid-related activity (e.g., protein synthesis), or any other cytotoxic effect.

Exemplary chemotherapeutic agents include, but are not limited to, alkylating agents (e.g., nitrogen mustards, such as chlorambucil, cyclophosphamide, isofamide, mechlorethamine, melphalan, and uracil mustard: aziridines, such as thiotepa: methanesulphonate esters, such as busulfan: nitroso ureas, such as carmustine, lomustine, and streptozocin: platinum complexes, such as cisplatin and carboplatin, oxaliplatin, nedaplatin, triplatin tetranitrate, phenanthriplatin, picoplatin, satraplatin and lipoplatin: bioreductive alkylators, such as mitomycin, procarbazine, dacarbazine and altretamine): DNA strand-breakage agents (e.g., bleomycin): topoisomerase II inhibitors (e.g., amsacrine, dactinomycin, daunorubicin, idarubicin, mitoxantrone, doxorubicin, etoposide, and teniposide): DNA minor groove binding agents (e.g., plicamydin); antimetabolites (e.g., folate antagonists such as methotrexate and trimetrexate: pyrimidine antagonists such as fluorouracil, fluorodeoxyuridine, CB3717, azacitidine, cytarabine, and floxuridine: purine antagonists, such as mercaptopurine, 6-thioguanine, fludarabine, pentostatin: asparginase; and ribonucleotide reductase inhibitors such as hydroxyurea): tubulin interactive agents (e.g., vincristine, vinblastine, and paclitaxel (Taxol)): hormonal agents (e.g., estrogens: conjugated estrogens: ethinyl estradiol: diethylstilbesterol: chlortrianisen: idenestrol: progestins, such as hydroxyprogesterone caproate, medroxyprogesterone, and megestrol; and androgens, such as testosterone, testosterone propionate, fluoxymesterone, and methyltestosterone): adrenal corticosteroids (e.g., prednisone, dexamethasone, methylprednisolone, and prednisolone); leutinizing hormone releasing agents or gonadotropin-releasing hormone agonists (e.g., leuprolide acetate and goserelin acetate); and anti-hormonal agents (e.g., tamoxifen, antiandrogen agents, such as flutamide: aromatase inhibitors, such as anastrozole, exemestane, and letrozole; and anti-adrenal agents, such as mitotane and aminoglutethimide).

The term “radiation therapy” or “radiotherapy” used herein refers to the medical use of ionizing radiation, generally as part of cancer treatment, to control or destroy malignant cells. It can also be used as part of adjuvant therapy to prevent tumour recurrence after surgery to remove a primary malignant tumour.

Radiation therapy may be delivered by a device placed outside the patient's body (external radiation therapy) or a source placed inside the patient's body (internal radiation therapy or brachytherapy), or intravenously or orally. It may also be delivered by a systemically delivered radioisotope. Radiation therapy can be planned and administered in conjunction with imaging based techniques, such as computed tomography (CT) or magnetic resonance imaging (MRI) to accurately determine the dose and location of radiation to be administered. In various embodiments, radiation therapy includes total body radiation therapy, conventional external beam radiation therapy, stereotactic radiosurgery, stereotactic radiation therapy, three-dimensional conformal radiation therapy, intensity modulated radiation therapy (IMRT), image-guided radiation therapy, tomotherapy and/or brachytherapy. In some examples, the radiation therapy includes stereotactic radiation therapy or intensity modulated radiation therapy (IMRT).

As used herein, “molecularly targeted therapy” or “molecularly targeted therapeutic agent” refers to a therapy that targets a particular class of proteins involved in cancer growth or signalling. In some examples, the further anti-cancer agent described herein is or comprises an inhibitor of a tyrosine kinase. The term “tyrosine kinase” refers to enzymes which are capable of transferring a phosphate group from ATP to a tyrosine residue in a protein. Phosphorylation of proteins by tyrosine kinases is an important mechanism in signal transduction for regulation of enzyme activity and cellular events such as cell survival or proliferation. In particular examples, the molecularly targeted therapy comprises one or more of a Human epidermal growth factor receptor 2 (HER2; also referred to as ErbB-2, NEU, HER-2 and CD340) inhibitor (e.g., trastuzumab, pertuzumab, neratinib, tucatinib), a PARP inhibitor (e.g., olaparib, talazoparib), a CDK4/6 inhibitor (e.g., abemaciclib), a PI3K inhibitor (e.g., alpelisib), a dual HER2/EGFR inhibitor (e.g., lapatinib), and a neurotrophic T receptor kinase (NTRK) inhibitor (e.g., entrectinib, larotrectinib).

Insofar as they relate to cancer, immunotherapy or immunotherapeutic agents use or modify the 30) immune mechanisms of a subject so as to promote or facilitate treatment of a cancer. In this regard, immunotherapy or immunotherapeutic agents used to treat cancer include cell-based therapies, antibody therapies (e.g., anti-PD1, anti-PDL1 or anti-CTLA4 antibodies) and cytokine therapies. These therapies all exploit the phenomenon that cancer cells often have subtly different molecules termed cancer antigens on their surface that can be detected by the immune system of the cancer subject. Accordingly, immunotherapy is used to provoke the immune system of a cancer patient into attacking the cancer's cells by using these cancer antigens as targets.

Non-limiting examples of immunotherapy or immunotherapeutic agents include adalimumab, alemtuzumab, basiliximab, belimumab, bevacizumab, BMS-936559, brentuximab, certolizumab, cituximab, daclizumab, eculizumab, ibritumomab, infliximab, ipilimumab, lambrolkizumab, mepolizumab, MPDL3280A muromonab, natalizumab, nivolumab, ofatumumab, omalizumab, pembrolizumab, pexelizumab, pidilizumab, rituximab, tocilizumab, tositumomab, trastuzumab, ustekinumab, abatacept, alefacept and denileukin diftitox. In particular examples, the immunotherapeutic agent is an immune checkpoint inhibitor, such as an anti-PD1 antibody (e.g., pidilizumab, nivolumab, lambrolkizumab, pembrolizumab), an anti-PDL1 antibody (e.g., BMS-936559, MPDL3280A) and/or an anti-CTLA4 antibody (e.g., ipilimumab).

### Lipid Biomarkers

As described herein, the inventors have found that the concentration levels of particular lipid biomarkers in blood or plasma samples from subjects can be diagnostic of breast cancer. Without being bound by any theory, it is believed that the lipid biomarkers are derived from extracellular vesicles, such as exosomes, present within the biological sample. It is also possible that other sources of lipid biomarkers that co-isolate with extracellular vesicles may contribute, such as apolipoproteins or lipid droplets.

As used herein, the term “lipid” refers to a group of organic compounds that has lipophilic or amphiphilic properties, including, but not limited to, bis(monoacylglycero)phosphates (BMP), cholesterol esters (CE), ceramides (Cer), diacylglycerols (DG or DAG), dihydroleukotriene B4 (DH-LTB4), fatty acids (FA), gangliosides A2 (GA2), gangliosides M3 (GM3), hexose ceramides (HexCer), dihexosylceramide (Hex2Cer), hexosyl dihydroceramide (HexDHCer), lactosylceramide (LacCer), lysophosphatidic acid (LysoPA or LPA), lysophosphatidylcholines (LysoPC or LPC), lysophosphatidylcholines-plasmalogens (LysoPC-pmg), lysophosphatidylethanolamines (LysoPE or LPE), lysophosphatidylethanolamines-plasmalogens (LysoPE-pmg), lysophosphatidylserines (LysoPS or LPS), lysophosphatidylinositols (LPI), monoacylglycerols (MAG), phosphatidylcholines (PC), phosphatidylcholines-plasmalogens (PC-pmg), phosphatidylethanolamines (PE), phosphatidylethanolamines-plasmalogens (PE-pmg), prostaglandin A1 (PGA1), prostaglandin B1 (PGB1), phosphatidylinositols (PI), phosphatidylserines (PS), sphingomyelins (SM), sphingosine, triacylglycerols (TG or TAG) and tetrahydro-12-keto-leukotriene B4 (TH-12-keto-LTB4).

The term “biomarker” as used herein refers to a lipid molecule whose levels are indicative or diagnostic of a subject having breast cancer. It will be appreciated that the term “biomarker” is intended to encompass all classes, forms (e.g., phosphorylated or oxidised forms), fragments (e.g., a lipid head group, a fatty acyl chain) and variants of a lipid biomarker, as are known in the art, such as those provided herein. It is also envisaged that the ether-linked lipids described herein (e.g., PC, PE, PS etc) encompass both alkyl-ether and alkenyl-ether forms thereof unless stated otherwise (e.g., the alkyl-ether and alkenyl-ether of PE(40:7e) include PE(O-40:7) and PE(P-40:6) respectively). To this end, the ‘O-’ prefix is used to indicate the presence of an alkyl ether substituent, whereas the “P-” prefix or “p” suffix is used for the alkenyl ether substituent.

For the methods, systems and kits described herein, the one or more lipid biomarkers described herein can be selected from one or more (e.g., 1, 2, 3, 4, 5, 6, 7, 8, 9, 10, 11, 12 etc) classes of lipids, such as Cer, DG, Hex2Cer, LPC, LPE, LPI, PC, PE, PI, PS, SM and TG.

In certain examples, the one or more lipid biomarkers comprise LPC(14:0) and/or PI(38:6). More particularly, the one or more lipid biomarkers may comprise LPC(14:0) and/or PI(18:2_20:4). In other examples, the one or more lipid biomarkers comprise LPC(14:0) and PI(38:6). More particularly, the one or more lipid biomarkers may comprise LPC(14:0) and PI(18:2_20:4).

In some examples, the one or more lipid biomarkers comprise LPC(14:0), or a fragment, variant or derivative thereof. In such examples, the one or more lipid biomarkers can optionally further include one or more other lipid biomarkers selected from the group consisting of Cer(d36:1), Cer(d42:0), Cer(d42:1), DG(34:2), Hex2Cer(d34:1), Hex2Cer(d42:2), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

In other examples, the one or more lipid biomarkers comprise PI(38:6), or a fragment, variant or derivative thereof. For such examples, the one or more lipid biomarkers can optionally further include one or more other lipid biomarkers selected from the group consisting of Cer(d36:1), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

In certain examples, the one or more lipid biomarkers comprise LPC(14:0) and PI(38:6), or a fragment, variant or derivative thereof. In such examples, the one or more lipid biomarkers can optionally further include one or more other lipid biomarkers selected from the group consisting of Cer(d36:1), Cer(d42:0), Cer(d42:1), DG(34:2), Hex2Cer(d34:1), Hex2Cer(d42:2), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

Suitably, the one or more lipid biomarkers are selected from the group consisting of LPC(14:0), PC(32:1), PC(38:5), PE(34:1), PS(38:4), SM(d36:2), SM(d38:4), TG(54:4), TG(56:1) and TG(58:2), or a fragment, variant or derivative thereof. In various examples, the one or more lipid biomarkers comprise or consist of LPC(14:0), PC(32:1), PC(38:5), PE(34:1), PS(38:4), SM(d36:2), SM(d38:4), TG(54:4), TG(56:1) and TG(58:2), or a fragment, variant or derivative thereof.

Suitably, the one or more lipid biomarkers described herein are selected from the group consisting of LPC(14:0), PC(32:1), PC(36:2), PC(38:5), PE(34:1), PI(34:1), PS(38:4), SM(d36:2), SM(d38:4), SM(d44:4), TG(52:3e), TG(53:4), TG(54:4), TG(54:5), TG(54:6), TG(56:1), TG(58:2) and TG(58:3), or a fragment, variant or derivative thereof. In certain examples the one or more lipid biomarkers comprise or consist of LPC(14:0), PC(32:1), PC(36:2), PC(38:5), PE(34:1), PI(34:1), PS(38:4), SM(d36:2), SM(d38:4), SM(d44:4), TG(52:3e), TG(53:4), TG(54:4), TG(54:5), TG(54:6), TG(56:1). TG(58:2) and TG(58:3), or a fragment, variant or derivative thereof. More particularly, the one or more lipid biomarkers can be selected from the group consisting of LPC(14:0), PE(34:1), PS(38:4), SM(d36:2), TG(52:3e) and TG(58:3), or a fragment, variant or derivative thereof. In some examples, the one or more lipid biomarkers comprise or consist of LPC(14:0), PE(34:1), PS(38:4), SM(d36:2), TG(52:3e) and TG(58:3), or a fragment, variant or derivative thereof.

Suitably, the one or more lipid biomarkers described herein are selected from the group consisting of LPC(14:0), LPE(22:6), PI(38:6), PE(34:2p), SM(d35:1), PS(38:4) and PS(38:4), or a fragment, variant or derivative thereof. In certain examples, the one or more lipid biomarkers comprise or consist of LPC(14:0), LPE(22:6), PI(38:6), PE(34:2p), SM(d35:1), PS(38:4) and PS(38:4), or a fragment, variant or derivative thereof.

Suitably, the one or more lipid biomarkers are selected from the group consisting of Cer(d42:1), LPC(14:0), LPC(16:0e), LPC(17:1), LPE(22:6), PE(34:2p). PE(36:4), PE(38:3p), PI(36:1), PI(38:6), PS(38:4), PS(40:6), SM(d33:1), SM(d35:1), SM(d41:2) and TG(57:1), or a fragment, variant or derivative thereof. In particular examples, the one or more lipid biomarkers comprise or consist of Cer(d42:1), LPC(14:0), LPC(16:0e), LPC(17:1), LPE(22:6), PE(34:2p), PE(36:4), PE(38:3p), PI(36:1), PI(38:6), PS(38:4), PS(40:6), SM(d33:1), SM(d35:1), SM(d41:2) and TG(57:1), or a fragment, variant or derivative thereof. In some examples, the one or more lipid biomarkers are selected from the group consisting of LPC(14:0), LPC(16:0e), LPE(22:6), PE(34:2p), PE(38:3p), PI(36:1), PI(38:6), PS(38:4), PS(40:6), SM(d33:1), SM(d35:1) and TG(57:1), or a fragment, variant or derivative thereof. In certain examples, the one or more lipid biomarkers comprise or consist of LPC(14:0), LPC(16:0e), LPE(22:6), PE(34:2p), PE(38:3p), PI(36:1), PI(38:6), PS(38:4), PS(40:6), SM(d33:1), SM(d35:1) and TG(57:1), or a fragment, variant or derivative thereof.

In some examples, the one or more lipid biomarkers comprise or consist of LPC(14:0), PS(38:4) and TG(57:1), or a fragment, variant or derivative thereof. In other examples, the one or more lipid biomarkers comprise or consist of LPC(16:0e), LPE(22:6) and PI(38:6), or a fragment, variant or derivative thereof. In particular examples, the one or more lipid biomarkers comprise or consist of PE(34:2p), PE(38:3p), PI(36:1), PI(38:6) and PS(40:6), or a fragment, variant or derivative thereof. In various examples, the one or more lipid biomarkers comprise or consist of LPC(14:0), PI(38:6), and SM(d33:1), or a fragment, variant or derivative thereof. In certain examples, the one or more lipid biomarkers comprise or consist of LPC(14:0), PI(38:6), and SM(d35:1), or a fragment, variant or derivative thereof. For the above examples, the one or more lipid biomarkers can optionally further include one or more other lipid biomarkers provided herein, such as those selected from the group consisting of LPC(14:0), LPC(16:0e), LPE(22:6), PE(34:2p), PE(38:3p), PI(36:1), PI(38:6), PS(38:4), PS(40:6), SM(d33:1), SM(d35:1) and TG(57:1), or a fragment, variant or derivative thereof.

Also provided are lipid “variants” such as naturally occurring variants, isobars and isomers (including stereoisomers) of the lipid biomarkers provided herein. To this end, it is further envisaged that the lipid biomarkers described herein may encompass a collection of one or more isomers thereof. For example, PC(32:1) is a lipid or lipid biomarker that is the collection of one or more phosphatidylcholine isomers that have 32 carbons in the acyl chain and one double bond in either of the two acyl chains. Exemplary isomers for the lipid biomarkers described herein are provided in the below table. Suitably, each of the lipid biomarker isomers have identical molecular weights. Although a lipid biomarker can encompass a total number of isomers thereof, a biological sample from a subject may only contain one isomer, two isomers, three isomers, four isomers, five isomers etc, or any number of isomers less than the total number of all possible isomers of said lipid biomarker. Accordingly, a lipid biomarker can refer to one or more of the isomers that make up the entire collection of possible isomers.

Table of isomers

| Lipid Biomarker | Isomers and Isobars | m/z* |
|---|---|---|
| Cer(d36:1) | Cer(d18:1_18:0), Cer(d18:0_18:1), Cer(d14:1_22:0), | 565.5434 |
| | Cer(d16:1_20:0), Cer(d18:0/18:1(9Z)) | |
| Cer(d42:0) | Cer(d18:0_24:0), Cer(d20:0_22:0) | 651.6529 |
| Cer(d42:1) | Cer(d18:1_24:0), Cer(d18:0_24:1) | 649.6373 |
| DG(34:2) | DG(17:1_17:1), DG(17:0_17:2), DG(16:1_18:1), | 592.5067 |
| | DG(16:0_18:2), DG(12:0_22:2), DG(14:0_20:2), | |
| | DG(14:1_20:1), DG(16:0/_18:2(9Z,12Z)), | |
| | DG(18:2(9Z,12Z)_/16:0), DG(16:1(9Z)_/18:1(9Z)) | |
| Hex2Cer(d34:1) | Hex2Cer(d18:1_16:0), LacCer (d18:1_16:0), Manβ1-4Glcβ- | 861.6177 |
| | Cer(d18:1_16:0), Galα1-4Galβ-Cer(d18:1_16:0) | |
| Hex2Cer(d42:2) | Hex2Cer(d18:1_24:1), LacCer(d18:1/24:1(15Z)), Manβ1-4Glcβ- | 971.7273 |
| | Cer(d18:1/24:1(15Z)), Galα1-4Galβ-Cer(d18:1/24:1(15Z)) | |
| LPC(14:0) | LPC(14:0), LPC (1-14:0), LPC (2-14:0), LPE (1-17:0) | 467.3012 |
| LPC(16:0e) | LPC(16:0e), LPC (O-16:0) | 481.3532 |
| LPC(17:1) | LPC(17:1) | 507.3325 |
| LPC(18:3) | LPC(18:3), LPC(1-18:3(6Z,9Z,12Z)), LPC(1-18:3(9Z,12Z,15Z)) | 517.3168 |
| LPE(22:6) | LPE(22:6), PE(1-22:6(4Z,7Z,10Z,13Z,16Z,19Z)), PE(2- | 525.2855 |
| | 22:6(4Z,7Z,10Z,13Z,16Z,19Z)) | |
| LPI(20:4) | LPI(20:4) | 620.2962 |
| PC(32:1) | PC(16:0_16:1), PC(14:0_18:1), PC(18:1_14:0), PC(12:0_20:1), | 731.5465 |
| | PC(13:0_19:1), PC(14:1_18:0), PC(15:0_17:1), PC(15:1_17:0), | |
| | PC(16:1_16:0), PC(17:0_15:1), PC(17:1_15:0), PC(18:0_14:1), | |
| | PC(19:1_13:0), PC(20:1_12:0), PC(16:0/16:1(9Z)), | |
| | PC(16:1(9Z)/16:0), PC(14:0/18:1(9Z)) | |
| PC(34:1) | PC(16:0_18:1), PC(18:0_16:1), PC(18:1_16:0), PC(12:0_22:1), | 759.5778 |
| | PC(14:0_20:1), PC(14:1_20:0), PC(15:0_19:1), PC(15:1_19:0), | |
| | PC(16:1_18:0), PC(17:0_17:1), PC(17:1_17:0), PC(19:0_15:1), | |
| | PC(19:1_15:0), PC(20:0_14:1), PC(20:1_14:0), PC(22:1_12:0) | |
| PC(35:4) | PC(15:0_20:4), PC(18:2_17:2), PC(13:0_22:4), PC(15:1_20:3), | 767.5465 |
| | PC(17:0_18:4), PC(17:1_18:3), PC(17:2_18:2), PC(15:1_20:3), | |
| | PC(22:4_13:0) | |
| PC(36:2) | PC(18:0_18:2), PC(18:1_18:1), PC(18:2_18:0), PC(14:0_22:2), | 785.5935 |
| | PC(14:1_22:1), PC(16:0_20:2), PC(16:1_20:1), PC(17:1_19:1), | |
| | PC(17:2_19:0), PC(19:0_17:2), PC(19:1_17:1), PC(20:1_16:1), | |
| | PC(20:2_16:0), PC(22:1_14:1), PC(22:2_14:0), | |
| | PC(18:0/18:2(9Z,12Z)), PC(18:1(9Z)/18:1(9Z)), 18:1 (46-Cis) | |
| | PC, 18:1(11-cis) PC | |
| PC(36:3) | PC(18:1_18:2), PC(16:0_20:3), PC(14:1_22:2), PC(16:1_20:2), | 783.5778 |
| | PC(17:2_19:1), PC(18:0_18:3), PC(18:2_18:1), PC(18:3_18:0), | |
| | PC(19:1_17:2), PC(20:2_16:1), PC(20:3_16:0), PC(22:2_14:1), | |
| | PC(18:1(9Z)/18:2(9Z,12Z)), PC(18:2(9Z,12Z)/18:1(9Z)), | |
| | PC(18:1(11Z)/18:2(9Z,12Z)) | |
| PC(37:4) | PC(17:0_20:4), PC(15:0_22:4), PC(17:1_20:3, PC(17:2_20:2), | 795.5778 |
| | PC(18:3_19:1), PC(18:4_19:0), PC(19:0_18:4), PC(19:1_18:3), | |
| | PC(20:2_17:2), PC(20:3_17:1), PC(20:4_17:0), PC(22:4_15:0), | |
| | PC(17:0/20:4(5Z,8Z,11Z,14Z)), PC(20:4(5Z,8Z,11Z,14Z)/17:0), | |
| | PC(17:1(9Z)/20:3(8Z,11Z,14Z)) | |
| PC(38:5) | PC(18:0_20:5), PC(18:1_20:4), PC(20:3_18:2), PC(16:0_22:5), | 807.5778 |
| | PC(16:1_22:4), PC(18:2_20:3), PC(18:3_20:2), PC(18:4_20:1), | |
| | PC(20:1_18:4), PC(20:2_18:3), PC(20:4_18:1), PC(20:5_18:0), | |
| | PC(22:4_16:1), PC(18:2(9Z,12Z)/20:3(5Z,8Z,11Z)), | |
| | PC(18:2(9Z,12Z)/20:3(8Z,11Z,14Z)), | |
| | PC(20:3(8Z,11Z,14Z)/18:2(9Z,12Z)), | |
| | PC(18:1(9Z)/20:4(5Z,8Z,11Z,14Z)), | |
| | PC(18:1(9Z)/20:4(8Z,11Z,14Z,17Z)), | |
| | PC(18:1(11Z)/20:4(5Z,8Z,11Z,14Z)), | |
| | PC(18:0/20:5(5Z,8Z,11Z,14Z,17Z)), | |
| | PC(18:0/20:5(9Z,11Z,13Z,15Z,17Z)), | |
| | PC(20:5(5Z,8Z,11Z,14Z,17Z)/18:0) | |
| PE(34:1) | PE(16:0_18:1), PE(18:1_16:0), PE(12:0_22:1), PE(14:0_20:1), | 717.5309 |
| | PE(14:1_20:0), PE(15:0_19:1), PE(15:1_19:0), PE(16:1_18:0), | |
| | PE(17:0_17:1), PE(17:1_17:0), PE(19:0_15:1), PE(19:1_15:0), | |
| | PE(20:0_14:1), PE(20:1_14:0), PE(22:1_12:0), PE(18:0_16:1), | |
| | PE(16:0/18:1(9Z)), PE(16:0/18:1(11Z)), PE(18:1(9Z)/16:0) | |
| PE(34:2p)/PE(34:3e)/ | PE(16:0p_18:2), PE(O-16:0_18:3), PE(P-16:0/18:2(9Z,12Z)), | 699.5203 |
| PE(O-34:3) | PE(O-16:0/18:3(9Z,12Z,15Z)), PE(O-16:0/18:3(6Z,9Z,12Z)) | |
| PE(36:4) | PE(16:0_20:4), PE(18:2/18:2), PE(14:0/22:4), PE(16:1/20:3), | 739.5152 |
| | PE(18:1/18:3), PE(18:3/18:1), PE(18:4/18:0), PE(20:3/16:1), | |
| | PE(22:4/14:0), PE(20:4/16:0), PE(18:0/18:4) | |
| PE(38:3p)/PE(O- | PE(20:1p_18:2), PE(P-18:0_20:3), PE(O-18:0_20:4), PE(O- | 753.5672 |
| 38:4)/PE(38:4e) | 20:0_18:4), PE(O-16:0_22:4), PE(P-20:1/18:2(9Z,12Z)), PE(P- | |
| | 20:0/18:3(9Z,12Z,15Z)), PE(P-20:0/18:3(6Z,9Z,12Z)) | |
| PE(38:4) | PE(18:0_20:4), PE(20:0_18:4), PE(18:1_20:3), PE(16:0_22:4), | 767.5465 |
| | PE(18:2_20:2), PE(18:3_20:1), PE(18:4_20:0), PE(20:1_18:3), | |
| | PE(20:2_18:2), PE(20:3_18:1), PE(20:4_18:0), PE(22:4_16:0) | |
| PE(O-38:5)/ | PE(18:1e_20:4), PE(O-16:0_22:5), PE(O-18:0_20:5) | 751.5516 |
| PE(38:4p)/PE | | |
| (38:5e) | | |
| PE(O-40:6) | PE(18:1e_22:5), PE(18:1p_22:4), PE(20:0p_20:5), | 777.5672 |
| (PE(40:6e)/ | | |
| PE(40:5p)) | | |
| PI(32:1) | PI(16:0_16:1), PI(12:0_20:1), PI(13:0_19:1), PI(14:1_18:0), | 808.5102 |
| | PI(15:0_17:1), PI(15:1_17:0), PI(16:1_16:0), PI(17:0_15:1), | |
| | PI(17:1_15:0), PI(18:0_14:1), PI(19:1_13:0), PI(20:1_12:0), | |
| | PI(18:1_14:0), PI(14:0_18:1) | |
| PI(34:1) | PI(16:0_18:1), PI(12:0_22:1), PI(14:0_20:1), PI(14:1_20:0), | 836.5415 |
| | PI(15:0_19:1), PI(15:1_19:0), PI(16:1_18:0), PI(17:0_17:1), | |
| | PI(17:1_17:0), PI(19:0_15:1), PI(19:1_15:0), PI(20:0_14:1), | |
| | PI(20:1_14:0), PI(22:1_12:0), PI(18:0_16:1), PI(18:1_16:0), | |
| | PI(16:0/18:1(9Z)), PI(18:1(9Z)/16:0) | |
| PI(36:1) | PI(18:0_18:1), PI(14:0_22:1), PI(14:1_22:0), PI(15:1_21:0), | 864.5728 |
| | PI(16:1_20:0), PI(17:0_19:1), PI(17:1_19:0), PI(19:0_17:1), | |
| | PI(19:1_17:0), PI(20:0_16:1), PI(20:1_16:0), PI(21:0_15:1), | |
| | PI(22:0_14:1), PI(22:1_14:0), PI(18:1_18:0), PI(16:0_20:1), | |
| | PI(18:0/18:1(9Z)), PI(18:1(9Z)/18:0) | |
| PI(38:6) | PI(16:0_22:6), PI(18:2_20:4), PI(18:3_20:3), PI(18:4_20:2), | 882.5258 |
| | PI(20:2_18:4), PI(20:3_18:3), PI(20:4_18:2), PI(22:6_16:0), | |
| | PI(20:5_18:1), PI(18:1_20:5), | |
| | PI(18:2(9Z,12Z)/20:4(5Z,8Z,11Z,14Z)), | |
| | PI(20:4(5Z,8Z,11Z,14Z)/18:2(9Z,12Z)), | |
| | PI(16:0/22:6(4Z,7Z,10Z,13Z,16Z,19Z)), | |
| | PI(22:6(4Z,7Z,10Z,13Z,16Z,19Z)/16:0) | |
| PS(38:4) | PS(18:0_20:4), PS(18:1_20:3), PS(18:2_20:2), PS(18:3_20:1), | 811.5363 |
| | PS(18:4_20:0), PS(20:1_18:3), PS(20:2_18:2), PS(20:3_18:1), | |
| | PS(20:4_18:0), PS(22:4_16:0), PS(20:0_18:4), PS(16:0_22:4), | |
| | PS(18:0/20:4(5Z,8Z,11Z,14Z)), PS(20:4(5Z,8Z,11Z,14Z)/18:0) | |
| PS(40:6) | PS(18:0_22:6), PS(18:2_22:4), PS(18:4_22:2), PS(20:1_20:5), | 835.5363 |
| | PS(20:2_20:4), PS(20:3_20:3), PS(20:4_20:2), PS(20:5_20:1), | |
| | PS(22:2_18:4), PS(22:4_18:2), PS(22:6_18:0), | |
| | PS(18:0/22:6(4Z,7Z,10Z,13Z,16Z,19Z)), | |
| | PS(22:6(4Z,7Z,10Z,13Z,16Z,19Z)/18:0) | |
| PS(40:7) | PS(20:3_20:4), PS(18:1_22:6), PS(18:3_22:4), PS(20:2_20:5), | 833.5207 |
| | PS(20:4_20:3), PS(20:5_20:2), PS(22:4_18:3), PS(22:6_18:1) | |
| SM(d32:2) | SM(d18:2_14:0) | 672.5206 |
| SM(d33:1) | SM(d18:1_15:0), SM(d16:1_17:0) | 688.5519 |
| SM(d35:1) | SM(d18:1_17:0), SM(d19:1_16:0) | 716.5832 |
| SM(d36:1) | SM(d20:1_16:0), SM(d18:1_18:0), SM(d18:0_18:1), | 730.5989 |
| | SM(d16:1_20:0) | |
| SM(d36:2) | SM(d18:0_18:2), SM(d18:1_18:1), SM(d16:1_20:1), | 728.5832 |
| | SM(d18:2_18:0), SM(d19:1_17:1), SM(d18:0/18:2(9Z,12Z)), | |
| | SM(d18:1/18:1(9Z)) | |
| SM(d38:4) | SM(d38:4), SM(d18:1_20:3), SM(d20:1_18:3), SM(d18:2_20:2) | 752.5832 |
| SM(d40:3) | SM(d18:2_22:1), SM(d18:2(4E, 14Z)/22:1(13Z)) | 782.6302 |
| SM(d41:2) | SM(d18:1_23:1), SM(d17:1_24:1) | 798.6615 |
| SM(d41:3) | SM(d41:3), SM(d18:2(4E, 14Z)/23:1(13Z)) | 796.6458 |
| SM(d42:1) | SM(d18:1_24:0), SM(d18:0_24:1) | 814.6928 |
| SM(d42:4) | SM(d18:1_24:3), SM(d18:2_24:2), | 808.6458 |
| | SM(d18:2(4E, 14Z)/24:2(11Z,14Z)), | |
| | SM(d18:1(4E)/24:3(8Z,11Z,14Z)) | |
| SM(d44:4) | SM(d20:0_24:4), SM(d20:0/24:4(5Z,8Z,11Z,14Z)) | 836.6771 |
| TG(52:3e) | TG(16:0e_18:1_18:2), TG(O-16:0_18:2_18:1), TG(O- | 842.7727 |
| | 16:0/18:1(9Z)/18:2(9Z,12Z)), TG(O- | |
| | 16:0/18:2(9Z,12Z)/18:1(9Z)) | |
| TG(53:4) | TG(17:0_18:2_18:2), TG(17:2_18:1_18:1), | 868.752 |
| | TG(17:1_18:1_18:2), TG(17:2_18:0_18:2), | |
| | TG(17:0_18:1_18:3), TG(17:1_18:0_18:3), | |
| | TG(16:1_17:2_20:1), TG(16:0_17:2_20:2), | |
| | TG(16:1_17:1_20:2), TG(16:0_17:1_20:3), | |
| | TG(16:1_17:0_20:3), TG(16:0_17:0_20:4), | |
| | TG(17:2_17:2_19:0), TG(16:1_18:3_19:0), | |
| | TG(13:0_20:2_20:2), TG(12:0_19:0_22:4), | |
| | TG(12:0_19:1_22:3), TG(12:0_20:4_21:0), | |
| | TG(13:0_18:0_22:4), TG(13:0_18:1_22:3), | |
| | TG(13:0_18:2_22:2), TG(13:0_18:3_22:1), | |
| | TG(13:0_18:3_22:1), TG(13:0_18:4_22:0), | |
| | TG(13:0_20:0_20:4), TG(13:0_20:1_20:3), | |
| | TG(14:0_17:0_22:4), TG(14:0_17:1_22:3), | |
| | TG(14:0_17:2_22:2), TG(14:0_18:4_21:0), | |
| | TG(14:0_19:0_20:4), TG(14:0_19:1_20:3), | |
| | TG(14:1_17:0_22:3), TG(14:1_17:1_22:2), | |
| | TG(14:1_17:2_22:1), TG(14:1_18:3_21:0), | |
| | TG(14:1_18:3_21:0), TG(14:1_19:0_20:3), | |
| | TG(14:1_19:1_20:2), TG(15:0_16:0_22:4), | |
| | TG(15:0_16:1_22:3), TG(15:0_18:0_20:4), | |
| | TG(15:0_18:1_20:3), TG(15:0_18:2_20:2), | |
| | TG(15:0_18:3_20:1), TG(15:0_18:4_20:0), | |
| | TG(15:1_16:0_22:3), TG(15:1_16:1_22:2), | |
| | TG(15:1_18:0_20:3), TG(15:1_18:1_20:2), | |
| | TG(15:1_18:2_20:1), TG(15:1_18:3_20:0), | |
| | TG(16:0_18:3_19:1), TG(16:0_18:4_19:0), | |
| | TG(16:1_18:2_19:1), TG(16:1_18:3_19:0), | |
| | TG(17:0_18:0_18:4), TG(17:0_18:1_18:3), | |
| | TG(17:1_17:2_19:1), TG(17:1_18:0_18:3), | |
| | TG(17:0/18:2(9Z,12Z)/18:2(9Z,12Z)), | |
| | TG(18:2(9Z,12Z)/17:0/18:2(9Z,12Z)), | |
| | TG(17:0/18:1(9Z)/18:3(6Z,9Z,12Z)) | |
| TG(54:3) | TG(18:1_18:1_18:1), TG(17:1_17:2_20:0), | 884.7833 |
| | TG(17:0_17:2_20:1), TG(17:1_17:1_20:1), | |
| | TG(17:0_17:1_20:2), TG(17:0_17:0_20:3), | |
| | TG(18:0_18:1_18:2), TG(18:0_18:0_18:3), | |
| | TG(16:0_18:3_20:0), TG(16:1_18:2_20:0), | |
| | TG(16:0_18:2_20:1), TG(16:1_18:1_20:1), | |
| | TG(16:0_18:1_20:2), TG(16:1_18:0_20:2), | |
| | TG(16:0_18:0_20:3), TG(17:0_18:3_19:0), | |
| | TG(17:1_18:2_19:0), TG(17:2_18:1_19:0), | |
| | TG(16:0_16:0_22:3), TG(16:1_16:1_22:1), | |
| | TG(16:1_17:2_21:0), TG(16:0_16:1_22:2), | |
| | TG(17:2_18:3_20:5), TG(18:1_18:1_18:1), | |
| | TG(18:1_18:1_18:1), TG(14:1_20:1_20:1), | |
| | TG(15:0_20:5_20:5), TG(16:1_16:1_22:1), | |
| | TG(16:1_19:1_19:1), TG(18:0_18:0_18:3), | |
| | TG(12:0_20:0_22:3), TG(12:0_20:1_22:2), | |
| | TG(12:0_20:2_22:1), TG(12:0_20:3_22:0), | |
| | TG(13:0_19:0_22:3), TG(13:0_19:1_22:2), | |
| | TG(13:0_20:3_21:0), TG(13:0_20:4_22:6), | |
| | TG(13:0_20:5_22:5), TG(14:0_18:0_22:3), | |
| | TG(14:0_18:1_22:2), TG(14:0_18:2_22:1), | |
| | TG(14:0_18:3_22:0), TG(14:0_18:3_22:0), | |
| | TG(14:0_20:0_20:3), TG(14:0_20:1_20:2), | |
| | TG(14:1_18:0_22:2), TG(14:1_18:1_22:1), | |
| | TG(14:1_18:2_22:0), TG(14:1_20:0_20:2), | |
| | TG(15:0_17:0_22:3), TG(15:0_17:1_22:2), | |
| | TG(15:0_17:2_22:1), TG(15:0_18:3_21:0), | |
| | TG(15:0_18:3_21:0), TG(15:0_19:0_20:3), | |
| | TG(15:0_19:1_20:2), TG(15:1_17:0_22:2), | |
| | TG(15:1_17:1_22:1), TG(15:1_17:2_22:0), | |
| | TG(15:1_18:2_21:0), TG(15:1_19:0_20:2), | |
| | TG(15:1_19:1_20:1), TG(16:0_18:3_20:0), | |
| | TG(17:0_18:2_19:1), TG(17:0_18:3_19:0), | |
| | TG(17:1_18:1_19:1), TG(17:2_18:0_19:1) | |
| TG(54:4) | TG(18:1_18:1_18:2), TG(17:2_17:2_20:0), | 882.7676 |
| | TG(17:1_17:1_20:2), TG(16:1_16:1_22:2), | |
| | TG(18:1_18:1_18:2), TG(17:0_17:0_20:4), | |
| | TG(16:0_16:0_22:4), TG(18:0_18:0_18:4), | |
| | TG(17:2_18:2_19:0), TG(17:2_18:1_19:1), | |
| | TG(17:1_18:3_19:0), TG(17:1_18:3_19:0), | |
| | TG(17:1_17:2_20:1), TG(17:1_18:2_19:1), | |
| | TG(16:1_18:3_20:0), TG(16:1_18:3_20:0), | |
| | TG(16:1_18:2_20:1), TG(16:1_18:1_20:2), | |
| | TG(16:1_18:0_20:3), TG(15:1_18:3_21:0), | |
| | TG(15:1_18:3_21:0), TG(15:1_17:2_22:1), | |
| | TG(15:1_17:1_22:2), TG(15:1_19:1_20:2), | |
| | TG(15:1_17:0_22:3), TG(15:1_19:0_20:3), | |
| | TG(14:1_20:1_20:2), TG(14:1_18:3_22:0), | |
| | TG(14:1_18:3_22:0), TG(14:1_18:2_22:1), | |
| | TG(14:1_18:1_22:2), TG(14:1_20:0_20:3), | |
| | TG(14:1_18:0_22:3), TG(12:0_20:2_22:2), | |
| | TG(12:0_20:1_22:3), TG(12:0_20:4_22:0), | |
| | TG(12:0_20:3_22:1), TG(12:0_20:0_22:4), | |
| | TG(17:0_18:4_19:0), TG(17:0_18:3_19:1), | |
| | TG(17:0_18:3_19:1), TG(17:0_17:2_20:2), | |
| | TG(17:0_17:1_20:3), TG(16:0_18:4_20:0), | |
| | TG(16:0_18:3_20:1), TG(16:0_18:3_20:1), | |
| | TG(16:0_18:2_20:2), TG(16:0_16:1_22:3), | |
| | TG(16:0_18:1_20:3), TG(16:0_18:0_20:4), | |
| | TG(18:0_18:2_18:2), TG(18:0_18:1_18:3), | |
| | TG(18:0_18:1_18:3), TG(15:0_18:4_21:0), | |
| | TG(15:0_17:2_22:2), TG(15:0_17:1_22:3), | |
| | TG(15:0_19:1_20:3), TG(15:0_17:0_22:4), | |
| | TG(15:0_19:0_20:4), TG(14:0_20:1_20:3), | |
| | TG(14:0_20:2_20:2), TG(14:0_18:4_22:0), | |
| | TG(14:0_18:3_22:1), TG(14:0_18:3_22:1), | |
| | TG(14:0_18:2_22:2), TG(14:0_18:1_22:3), | |
| | TG(14:0_20:0_20:4), TG(14:0_18:0_22:4), | |
| | TG(13:0_20:4_21:0), TG(13:0_19:1_22:3), | |
| | TG(13:0_19:0_22:4), TG(18:1(9Z)/18:1(9Z)/18:2(9Z,12Z)), | |
| | TG(18:1(9Z)/18:2(9Z,12Z)/18:1(9Z)), | |
| | TG(18:0/18:1(9Z)/18:3(6Z,9Z,12Z)) | |
| TG(54:5) | TG(18:1_18:2_18:2), TG(18:2_18:2_18:1), | 880.752 |
| | TG(17:2_17:2_20:1), TG(17:1_17:1_20:3), | |
| | TG(16:1_16:1_22:3), TG(18:1_18:1_18:3), | |
| | TG(18:1_18:1_18:3), TG(17:0_17:0_20:5), | |
| | TG(16:0_16:0_22:5), TG(17:2_18:3_19:0), | |
| | TG(17:2_18:3_19:0), TG(17:2_18:2_19:1), | |
| | TG(17:1_18:4_19:0), TG(17:1_18:3_19:1), | |
| | TG(17:1_18:3_19:1), TG(17:1_17:2_20:2), | |
| | TG(16:1_18:4_20:0), TG(16:1_18:3_20:1), | |
| | TG(16:1_18:3_20:1), TG(16:1_18:2_20:2), | |
| | TG(16:1_18:1_20:3), TG(16:1_18:0_20:4), | |
| | TG(18:1_18:2_18:2), TG(15:1_18:4_21:0), | |
| | TG(15:1_17:2_22:2), TG(15:1_17:1_22:3), | |
| | TG(15:1_19:1_20:3), TG(15:1_17:0_22:4), | |
| | TG(15:1_19:0_20:4), TG(14:1_20:1_20:3), | |
| | TG(14:1_20:2_20:2), TG(14:1_18:4_22:0), | |
| | TG(14:1_18:3_22:1), TG(14:1_18:3_22:1), | |
| | TG(14:1_18:2_22:2), TG(14:1_18:1_22:3), | |
| | TG(14:1_20:0_20:4), TG(14:1_18:0_22:4), | |
| | TG(12:0_20:2_22:3), TG(12:0_20:1_22:4), | |
| | TG(12:0_20:5_22:0), TG(12:0_20:4_22:1), | |
| | TG(12:0_20:3_22:2), TG(12:0_20:0_22:5), | |
| | TG(17:0_18:4_19:1), TG(17:0_17:2_20:3), | |
| | TG(17:0_17:1_20:4), TG(16:0_18:4_20:1), | |
| | TG(16:0_18:3_20:2), TG(16:0_18:3_20:2), | |
| | TG(16:0_18:2_20:3), TG(16:0_16:1_22:4), | |
| | TG(16:0_18:1_20:4), TG(16:0_18:0_20:5), | |
| | TG(18:0_18:2_18:3), TG(18:0_18:2_18:3), | |
| | TG(18:0_18:1_18:4), TG(15:0_17:2_22:3), | |
| | TG(15:0_17:1_22:4), TG(15:0_19:1_20:4), | |
| | TG(15:0_17:0_22:5), TG(15:0_19:0_20:5), | |
| | TG(14:0_20:2_20:3), TG(14:0_20:1_20:4), | |
| | TG(14:0_18:4_22:1), TG(14:0_18:3_22:2), | |
| | TG(14:0_18:3_22:2), TG(14:0_18:2_22:3), | |
| | TG(14:0_18:1_22:4), TG(14:0_20:0_20:5), | |
| | TG(14:0_18:0_22:5), TG(13:0_20:5_21:0), | |
| | TG(13:0_19:1_22:4), TG(13:0_19:0_22:5), | |
| | TG(18:1(9Z)/18:2(9Z,12Z)/18:2(9Z,12Z)), | |
| | TG(18:2(9Z,12Z)/18:1(9Z)/18:2(9Z,12Z)), | |
| | TG(18:1(9Z)/18:1(9Z)/18:3(9Z,12Z,15Z)) | |
| TG(54:6) | TG(18:1_18:2_18:3), TG(17:2_17:2_20:2), | 878.7363 |
| | TG(17:1_17:1_20:4), TG(16:1_16:1_22:4), | |
| | TG(18:1_18:1_18:4), TG(16:0_16:0_22:6), | |
| | TG(17:2_18:4_19:0), TG(17:2_18:3_19:1), | |
| | TG(17:2_18:3_19:1), TG(17:1_18:4_19:1), | |
| | TG(17:1_17:2_20:3), TG(16:1_18:4_20:1), | |
| | TG(16:1_18:3_20:2), TG(16:1_18:3_20:2), | |
| | TG(16:1_18:2_20:3), TG(16:1_18:1_20:4), | |
| | TG(16:1_18:0_20:5), TG(18:1_18:2_18:3), | |
| | TG(18:1_18:2_18:3), TG(15:1_17:2_22:3), | |
| | TG(15:1_17:1_22:4), TG(15:1_19:1_20:4), | |
| | TG(15:1_17:0_22:5), TG(15:1_19:0_20:5), | |
| | TG(14:1_20:2_20:3), TG(14:1_20:1_20:4), | |
| | TG(14:1_18:4_22:1), TG(14:1_18:3_22:2), | |
| | TG(14:1_18:3_22:2), TG(14:1_18:2_22:3), | |
| | TG(14:1_18:1_22:4), TG(14:1_20:0_20:5), | |
| | TG(14:1_18:0_22:5), TG(12:0_20:2_22:4), | |
| | TG(12:0_20:1_22:5), TG(12:0_20:5_22:1), | |
| | TG(12:0_20:4_22:2), TG(12:0_20:3_22:3), | |
| | TG(12:0_20:0_22:6), TG(17:0_17:2_20:4), | |
| | TG(17:0_17:1_20:5), TG(16:0_18:4_20:2), | |
| | TG(16:0_18:3_20:3), TG(16:0_18:3_20:3), | |
| | TG(16:0_18:2_20:4), TG(16:0_16:1_22:5), | |
| | TG(16:0_18:1_20:5), TG(18:0_18:3_18:3), | |
| | TG(18:0_18:3_18:3), TG(18:0_18:3_18:3), | |
| | TG(18:0_18:2_18:4), TG(15:0_17:2_22:4), | |
| | TG(15:0_17:1_22:5), TG(15:0_19:1_20:5), | |
| | TG(15:0_17:0_22:6), TG(14:0_20:2_20:4), | |
| | TG(14:0_20:1_20:5), TG(14:0_20:3_20:3), | |
| | TG(14:0_18:4_22:2), TG(14:0_18:3_22:3), | |
| | TG(14:0_18:3_22:3), TG(14:0_18:2_22:4), | |
| | TG(14:0_18:1_22:5), TG(14:0_18:0_22:6), | |
| | TG(13:0_19:1_22:5), TG(13:0_19:0_22:6), | |
| | TG(18:1(9Z)/18:2(9Z,12Z)/18:3(6Z,9Z,12Z)), | |
| | TG(18:1(9Z)/18:3(6Z,9Z,12Z)/18:2(9Z,12Z)), | |
| | TG(18:3(6Z,9Z,12Z)/18:1(9Z)/18:2(9Z,12Z)) | |
| TG(56:1) | TG(16:0_18:1_22:0), TG(16:1_20:0_20:0), | 916.8459 |
| | TG(18:0_18:1_20:0), TG(16:0_20:0_20:1), | |
| | TG(18:0_18:0_20:1), TG(17:1_19:0_20:0), | |
| | TG(17:0_19:0_20:1), TG(17:0_17:1_22:0), | |
| | TG(17:0_17:0_22:1), TG(16:0_18:1_22:0), | |
| | TG(16:1_18:0_22:0), TG(16:0_18:0_22:1), | |
| | TG(18:1_19:0_19:0), TG(17:0_18:1_21:0), | |
| | TG(17:1_18:0_21:0), TG(16:1_19:0_21:0), | |
| | TG(14:1_21:0_21:0), TG(17:0_17:0_22:1), | |
| | TG(12:0_22:0_22:1), TG(13:0_21:0_22:1), | |
| | TG(14:0_20:0_22:1), TG(14:0_20:1_22:0), | |
| | TG(14:1_20:0_22:0), TG(15:0_19:0_22:1), | |
| | TG(15:0_19:1_22:0), TG(15:0_20:1_21:0), | |
| | TG(15:1_19:0_22:0), TG(15:1_20:0_21:0), | |
| | TG(16:0_18:0_22:1), TG(16:0_19:1_21:0), | |
| | TG(17:0_19:1_20:0), TG(18:0_19:0_19:1), | |
| | TG(16:0/18:1(9Z)/22:0), TG(18:1(9Z)/16:0/22:0) | |
| TG(57:1) | TG(16:0_18:1_23:0), TG(17:1_20:0_20:0), | 930.8615 |
| | TG(17:0_20:0_20:1), TG(18:1_19:0_20:0), | |
| | TG(18:0_19:0_20:1), TG(17:0_18:1_22:0), | |
| | TG(17:1_18:0_22:0), TG(17:0_18:0_22:1), | |
| | TG(16:0_20:1_21:0), TG(16:1_20:0_21:0), | |
| | TG(18:0_18:1_21:0), TG(16:1_19:0_22:0), | |
| | TG(16:0_19:0_22:1), TG(17:1_19:0_21:0), | |
| | TG(15:1_21:0_21:0), TG(19:0_19:0_19:1), | |
| | TG(13:0_22:0_22:1), TG(14:0_21:0_22:1), | |
| | TG(14:1_21:0_22:0), TG(15:0_20:0_22:1), | |
| | TG(15:0_20:1_22:0), TG(15:1_20:0_22:0), | |
| | TG(16:0_19:0_22:1), TG(16:0_19:1_22:0), | |
| | TG(17:0_18:0_22:1), TG(17:0_19:1_21:0), | |
| | TG(18:0_19:1_20:0), TG(16:0/18:1(9Z)/23:0), | |
| | TG(18:1(9Z)/16:0/23:0), TG(16:0/19:1(9Z)/22:0) | |
| TG(58:1) | TG(18:0_18:1_22:0), TG(19:0_19:0_20:1), | 944.8772 |
| | TG(18:0_18:0_22:1), TG(18:0_18:0_22:1), | |
| | TG(17:1_20:0_21:0), TG(17:1_19:0_22:0), | |
| | TG(16:1_21:0_21:0), TG(16:1_20:0_22:0), | |
| | TG(18:1_20:0_20:0), TG(18:1_19:0_21:0), | |
| | TG(15:1_21:0_22:0), TG(14:1_22:0_22:0), | |
| | TG(17:0_20:1_21:0), TG(17:0_19:1_22:0), | |
| | TG(17:0_19:0_22:1), TG(17:0_19:0_22:1), | |
| | TG(16:0_20:1_22:0), TG(16:0_20:0_22:1), | |
| | TG(16:0_20:0_22:1), TG(19:0_19:1_20:0), | |
| | TG(18:0_19:1_21:0), TG(18:0_18:1_22:0), | |
| | TG(18:0_20:0_20:1), TG(15:0_21:0_22:1), | |
| | TG(14:0_22:0_22:1), TG(18:0/18:1(9Z)/22:0), | |
| | TG(18:1(9Z)/18:0/22:0), TG(18:0/18:0/22:1(13Z)) | |
| TG(58:2) | TG(18:1_18:1_22:0), TG(18:2_20:0_20:0), | 942.8615 |
| | TG(18:0_20:1_20:1), TG(18:1_20:0_20:1), | |
| | TG(18:0_20:0_20:2), TG(16:0_20:2_22:0), | |
| | TG(16:1_20:1_22:0), TG(18:0_18:2_22:0), | |
| | TG(18:1_18:1_22:0), TG(16:0_20:1_22:1), | |
| | TG(16:1_20:0_22:1), TG(18:0_18:1_22:1), | |
| | TG(19:0_19:0_20:2), TG(17:0_20:2_21:0), | |
| | TG(17:1_20:1_21:0), TG(17:2_20:0_21:0), | |
| | TG(17:2_19:0_22:0), TG(17:1_19:0_22:1), | |
| | TG(16:0_20:0_22:2), TG(18:0_18:0_22:2), | |
| | TG(17:0_19:0_22:2), TG(18:2_19:0_21:0), | |
| | TG(19:1_19:1_20:0), TG(14:0_22:0_22:2), | |
| | TG(14:1_22:0_22:1), TG(15:0_21:0_22:2), | |
| | TG(15:1_21:0_22:1), TG(16:0_20:1_22:1), | |
| | TG(16:1_20:0_22:1), TG(17:0_19:1_22:1), | |
| | TG(17:1_19:0_22:1), TG(17:1_19:1_22:0), | |
| | TG(18:0_18:1_22:1), TG(18:1_19:1_21:0), | |
| | TG(19:0_19:1_20:1), TG(18:1(9Z)/18:1(9Z)/22:0), | |
| | TG(18:1(9Z)/22:0/18:1(9Z)), TG(18:0/18:1(9Z)/22:1(11Z)) | |
| TG(58:3) | TG(18:1_18:1_22:1), TG(18:3_20:0_20:0), | 940.8459 |
| | TG(18:1_20:1_20:1), TG(18:2_20:0_20:1), | |
| | TG(18:0_20:1_20:2), TG(18:1_20:0_20:2), | |
| | TG(18:0_20:0_20:3), TG(16:0_20:0_22:3), | |
| | TG(18:0_18:0_22:3), TG(16:0_20:3_22:0), | |
| | TG(16:1_20:2_22:0), TG(18:0_18:3_22:0), | |
| | TG(18:1_18:2_22:0), TG(16:0_20:2_22:1), | |
| | TG(16:1_20:1_22:1), TG(18:0_18:2_22:1), | |
| | TG(18:1_18:1_22:1), TG(17:0_19:0_22:3), | |
| | TG(19:0_19:0_20:3), TG(17:0_20:3_21:0), | |
| | TG(17:1_20:2_21:0), TG(17:2_20:1_21:0), | |
| | TG(17:2_19:0_22:1), TG(16:0_20:1_22:2), | |
| | TG(16:1_20:0_22:2), TG(18:0_18:1_22:2), | |
| | TG(17:1_19:0_22:2), TG(18:3_19:0_21:0), | |
| | TG(14:1_22:1_22:1), TG(18:1_18:1_22:1), | |
| | TG(18:3_20:0_20:0), TG(19:1_19:1_20:1), | |
| | TG(14:0_22:0_22:3), TG(14:0_22:1_22:2), | |
| | TG(14:1_22:0_22:2), TG(15:0_21:0_22:3), | |
| | TG(15:1_21:0_22:2), TG(16:0_20:2_22:1), | |
| | TG(16:1_20:1_22:1), TG(17:0_19:1_22:2), | |
| | TG(17:1_19:1_22:1), TG(17:2_19:0_22:1), | |
| | TG(17:2_19:1_22:0), TG(18:0_18:2_22:1), | |
| | TG(18:0_18:3_22:0), TG(18:2_19:1_21:0), | |
| | TG(18:3_19:0_21:0), TG(18:1(9Z)/18:1(9Z)/22:1(11Z)), | |
| | TG(18:1(9Z)/18:1(9Z)/22:1(13Z)) | |
| TG(59:2) | TG(18:1_18:1_23:0), TG(19:0_20:1_20:1), | 956.8772 |
| | TG(19:0_20:0_20:2), TG(17:0_20:2_22:0), | |
| | TG(17:1_20:1_22:0), TG(17:2_20:0_22:0), | |
| | TG(17:0_20:1_22:1), TG(17:1_20:0_22:1), | |
| | TG(17:0_20:0_22:2), TG(18:0_20:2_21:0), | |
| | TG(18:1_20:1_21:0), TG(18:2_20:0_21:0), | |
| | TG(18:2_19:0_22:0), TG(18:1_19:0_22:1), | |
| | TG(18:0_19:0_22:2), TG(16:1_21:0_22:1), | |
| | TG(17:2_21:0_21:0), TG(16:0_21:0_22:2), | |
| | TG(15:0_22:1_22:1), TG(19:1_19:1_21:0), | |
| | TG(15:0_22:0_22:2), TG(15:1_22:0_22:1), | |
| | TG(16:1_21:0_22:1), TG(17:0_20:1_22:1), | |
| | TG(17:1_20:0_22:1), TG(18:0_19:1_22:1), | |
| | TG(18:1_19:0_22:1), TG(18:1_19:1_22:0), | |
| | TG(19:1_20:0_20:1), | |
| TG(60:1) | TG(18:0_18:1_24:0), TG(20:0_20:0_20:1), | 972.9085 |
| | TG(18:0_20:1_22:0), TG(18:1_20:0_22:0), | |
| | TG(18:0_20:0_22:1), TG(16:1_22:0_22:0), | |
| | TG(16:0_22:0_22:1), TG(19:0_20:1_21:0), | |
| | TG(17:1_21:0_22:0), TG(17:0_21:0_22:1), | |
| | TG(19:0_19:0_22:1), TG(18:1_21:0_21:0), | |
| | TG(19:0_19:0_22:1), TG(16:0_22:0_22:1), | |
| | TG(17:0_21:0_22:1), TG(18:0_20:0_22:1), | |
| | TG(19:0_19:1_22:0), TG(19:1_20:0_21:0), | |
| | TG(18:0/18:1(9Z)/24:0), TG(18:1(9Z)/18:0/24:0). | |
| | TG(20:0/18:1(9Z)/22:0) | |
| TG(62:2) | TG(26:0_18:1_18:1), TG(20:0_20:2_22:0), | 998.9241 |
| | TG(20:1_20:1_22:0), TG(20:0_20:1_22:1), | |
| | TG(20:0_20:0_22:2), TG(18:2_22:0_22:0), | |
| | TG(18:0_22:1_22:1), TG(18:1_22:0_22:1), | |
| | TG(18:0_22:0_22:2), TG(20:2_21:0_21:0), | |
| | TG(19:0_21:0_22:2), TG(18:1_22:0_22:1), | |
| | TG(19:1_21:0_22:1), TG(20:0_20:1_22:1), | |
| | TG(18:0_22:1_22:1), TG(18:1(9Z)/18:1(9Z)/26:0), | |
| | TG(18:1(9Z)/26:0)/18:1(9Z), TG(18:1(9Z)/22:1(11Z)/22:0) | |

*as measured by the mass spectrometry methods described herein.

In particular examples, PE(O-40:6) comprises PE(40:5p) and PE(40:6e). As PE(O-40:6) encompasses both PE(40:5p) and PE(40:6e), for example, the one or more lipid biomarkers described herein can be selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5) (e.g., PE(38:4p) and PE(38:5e)), PE(40:5p), PE(40:6e), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

In particular examples, PE(O-38:5) comprises PE(38:4p) and PE(38:5e). As PE(O-38:5) encompasses both PE(38:4p) and PE(38:5e), for example, the one or more lipid biomarkers described herein can be selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(38:4p), PE(38:5e) PE(O-40:6) (e.g., PE(40:5p) and PE(40:6e)), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

In other examples, PE(34:2p) comprises the isomer PE(34:3e) and may be used interchangeably herein.

It is also envisaged that the lipid biomarkers described herein may encompass or be interchangeable with one or more isobars thereof. The term “isobar” typically refers to different lipids that have nearly or substantially the same mass (e.g., m/z ratio) and may not be distinguished from each other on the analytical platform used in their detection (e.g., for mass spectrometry, the different lipids in an isobar can elute at nearly the same time and have similar or the same quant ions, and thus cannot be distinguished).

Lipids can be defined according to the following equation: XXX(YY:ZZ), in which XXX is the abbreviation for the lipid class or group (in many instances indicating the lipid headgroup), YY is the number of carbons in the acyl chain and ZZ is the number of double bonds in the acyl chains. Similar notation (e.g., XXX(YY₁:ZZ₁_YY₂:ZZ₂) or XXX(YY₁:ZZ₁_YY₂:ZZ₂_YY₃_ZZ₃) may be used to define lipid isomers, wherein the numbers refer to the particular acyl chain of the lipid. It is envisaged, however, that the lipids defined herein may be identified by different naming annotations or nomenclature as are known in the art (see, e.g., Liebisch et al., J Lipid Res. 2013 June; 54(6): 1523-1530; Lipidomics Standards Initiative Consortium, Nat Metab, 2019 August; 1(8): 745-747).

It is also envisaged that the recited lipid biomarkers may additionally cover one or more further lipid biomarkers that behave similarly or equivalently (e.g., demonstrate a similar concentration profile) to said lipid biomarker. To this end, the lipid biomarker may demonstrate substantial collinearity with one or more further lipid biomarkers in terms of, for example, being diagnostic or indicative of breast cancer in a subject. Collinearity refers to a strong correlation or linear relationship between a pair of predictors (e.g., a pair of lipid biomarkers), and collinearity between multiple predictors is called multi-collinearity. As such, in some examples, the one or more lipid biomarkers comprises one or more further lipid biomarkers, such as those outlined in Examples 1 through 3 below; that demonstrate collinearity with one or more of the one or more lipid biomarkers recited in the examples provided herein. In other examples, the lipid biomarker demonstrates little or no collinearity with one or more further lipid biomarkers.

Also provided are fragments of the lipid biomarkers, inclusive of a lipid headgroup and an acyl chain or fragments thereof, that comprise less than 100% of an entire lipid biomarker molecule. In this regard, the skilled person will appreciate that MRM analysis of lipid biomarkers by mass spectrometry can include fragmenting lipids into their component parts (e.g., lipid headgroups and one or more acyl chains) so as to assist in identification and quantification of said lipid biomarker, as described in more detail below.

High-resolution accurate-mass MS (HRMS) may also be utilised to perform reliable and sensitive quantitative analyses of lipid biomarkers, similar to that of MRM (see Rochat, Trends in Analytical Chemistry, 2016 for review). Parallel reaction monitoring (PRM) is an ion monitoring technique based on high-resolution and high-precision mass spectrometry. PRM can be based on, for example, a Q Exactive Orbitrap™ (Thermo Scientific™) system or a Sciex 7500 (Sciex™) system as the representative quadrupole-high resolution mass spectrum platform. Unlike MRM, which monitors specific transitions at a time, the high resolution and mass accuracy of full-scan (MS1) and tandem mass spectrometry (MS/MS) scan of PRM can result in sufficient selectivity by monitoring all MS/MS fragment ions for each target precursor lipid.

Suitably, the level (e.g., concentration or expression level) of two or more of the lipid biomarkers (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 lipid biomarkers) provided herein are determined for the methods described herein. In some examples, the methods described herein include the step of determining the level or concentration of three or more lipid biomarkers described herein. In other examples, the methods described herein include the step of determining the level or concentration of four or more lipid biomarkers described herein. In certain examples, the methods described herein include the step of determining the level or concentration of five or more lipid biomarkers described herein. In some examples, the methods described herein include the step of determining the level or concentration of six or more lipid biomarkers described herein. In various examples, the methods described herein include the step of determining the level or concentration of seven or more lipid biomarkers described herein. In particular examples, the methods described herein include the step of determining the level or concentration of eight or more lipid biomarkers described herein. In some examples, the methods described herein include the step of determining the level or concentration of nine or more lipid biomarkers described herein. In other examples, the methods described herein include the step of determining the level or concentration of ten or more lipid biomarkers described herein. In certain examples, the methods described herein include the step of determining the level or concentration of eleven or more lipid biomarkers described herein. In some examples, the methods described herein include the step of determining the level or concentration of twelve or more lipid biomarkers described herein. In various examples, the methods described herein include the step of determining the level or concentration of thirteen or more lipid biomarkers described herein. In particular examples, the methods described herein include the step of determining the level or concentration of fourteen or more lipid biomarkers described herein. In various examples, the methods described herein include the step of determining the level or concentration of fifteen or more lipid biomarkers described herein. In particular examples, the methods described herein include the step of determining the level or concentration of sixteen or more lipid biomarkers described herein. In some examples, the methods described herein include the step of determining the level or concentration of seventeen or more lipid biomarkers described herein. In certain examples, the methods described herein include the step of determining the level or concentration of eighteen or more lipid biomarkers described herein. In particular examples, the methods described herein include the step of determining the level or concentration of nineteen or more lipid biomarkers described herein. In various examples, the methods described herein include the step of determining the level or concentration of twenty or more lipid biomarkers described herein.

In one example, the methods of the present disclosure include the step of determining a level of Cer(d36:1) and at least one further lipid biomarker described herein (e.g., Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), and fragments, variants or derivatives thereof).

In one example, the methods of the present disclosure include the step of determining a level of Cer(d42:0) and at least one further lipid biomarker described herein.

In one example, the methods of the present disclosure include the step of determining a level of Cer(d42:1) and at least one further lipid biomarker described herein.

In one example, the methods of the present disclosure include the step of determining a level of DG(34:2) and at least one further lipid biomarker described herein.

In one example, the methods of the present disclosure include the step of determining a level of Hex2Cer(d34:1) and at least one further lipid biomarker described herein.

In one example, the methods of the present disclosure include the step of determining a level of Hex2Cer(d42:2) and at least one further lipid biomarker described herein.

In one example, the methods of the present disclosure include the step of determining a level of LPC(14:0) and at least one further lipid biomarker described herein.

In one example, the methods of the present disclosure include the step of determining a level of LPC(16:0e) and at least one further lipid biomarker described herein.

In one example, the methods of the present disclosure include the step of determining a level of LPC(17:1) and at least one further lipid biomarker described herein.

In one example, the methods of the present disclosure include the step of determining a level of LPC(18:3) and at least one further lipid biomarker described herein.

In one example, the methods of the present disclosure include the step of determining a level of LPE(22:6) and at least one further lipid biomarker described herein.

In one example, the methods of the present disclosure include the step of determining a level of LPI(20:4) and at least one further lipid biomarker described herein.

In one example, the methods of the present disclosure include the step of determining a level of PC(32:1) and at least one further lipid biomarker described herein.

In one example, the methods of the present disclosure include the step of determining a level of PC(34:1) and at least one further lipid biomarker described herein.

In one example, the methods of the present disclosure include the step of determining a level of PC(35:4) and at least one further lipid biomarker described herein.

In one example, the methods of the present disclosure include the step of determining a level of PC(36:2) and at least one further lipid biomarker described herein.

In one example, the methods of the present disclosure include the step of determining a level of PC(36:3) and at least one further lipid biomarker described herein.

In one example, the methods of the present disclosure include the step of determining a level of PC(37:4) and at least one further lipid biomarker described herein.

In one example, the methods of the present disclosure include the step of determining a level of PC(38:5) and at least one further lipid biomarker described herein.

In one example, the methods of the present disclosure include the step of determining a level of PE(34:1) and at least one further lipid biomarker described herein.

In one example, the methods of the present disclosure include the step of determining a level of PE(34:2p) and at least one further lipid biomarker described herein.

In one example, the methods of the present disclosure include the step of determining a level of PE(36:4) and at least one further lipid biomarker described herein.

In one example, the methods of the present disclosure include the step of determining a level of PE(38:3p) and at least one further lipid biomarker described herein.

In one example, the methods of the present disclosure include the step of determining a level of PE(38:4) and at least one further lipid biomarker described herein.

In one example, the methods of the present disclosure include the step of determining a level of PE(O-38:5) (e.g., PE(38:4p) and/or PE(38:5e)) and at least one further lipid biomarker described herein. More particularly, the methods of the present disclosure can include the step of determining a level of PE(38:4p) and at least one further lipid biomarker described herein. More particularly, the methods of the present disclosure can include the step of determining a level of PE(38:5e) and at least one further lipid biomarker described herein.

In one example, the methods of the present disclosure include the step of determining a level of PE(O-40:6) (e.g., PE(40:5p) and/or PE(40:6e)) and at least one further lipid biomarker described herein. More particularly, the methods of the present disclosure can include the step of determining a level of PE(40:5p) and at least one further lipid biomarker described herein. More particularly, the methods of the present disclosure can include the step of determining a level of PE(40:6e) and at least one further lipid biomarker described herein.

In one example, the methods of the present disclosure include the step of determining a level of PI(32:1) and at least one further lipid biomarker described herein.

In one example, the methods of the present disclosure include the step of determining a level of PI(34:1) and at least one further lipid biomarker described herein.

In one example, the methods of the present disclosure include the step of determining a level of PI(36:1) and at least one further lipid biomarker described herein.

In one example, the methods of the present disclosure include the step of determining a level of PI(38:6) and at least one further lipid biomarker described herein.

In one example, the methods of the present disclosure include the step of determining a level of PS(38:4) and at least one further lipid biomarker described herein.

In one example, the methods of the present disclosure include the step of determining a level of PS(40:6) and at least one further lipid biomarker described herein.

In one example, the methods of the present disclosure include the step of determining a level of PS(40:7) and at least one further lipid biomarker described herein.

In one example, the methods of the present disclosure include the step of determining a level of SM(d32:2) and at least one further lipid biomarker described herein.

In one example, the methods of the present disclosure include the step of determining a level of SM(d33:1) and at least one further lipid biomarker described herein.

In one example, the methods of the present disclosure include the step of determining a level of SM(d35:1) and at least one further lipid biomarker described herein.

In one example, the methods of the present disclosure include the step of determining a level of SM(d36:1) and at least one further lipid biomarker described herein.

In one example, the methods of the present disclosure include the step of determining a level of SM(d36:2) and at least one further lipid biomarker described herein.

In one example, the methods of the present disclosure include the step of determining a level of SM(d38:4) and at least one further lipid biomarker described herein.

In one example, the methods of the present disclosure include the step of determining a level of SM(d40:3) and at least one further lipid biomarker described herein.

In one example, the methods of the present disclosure include the step of determining a level of SM(d41:2) and at least one further lipid biomarker described herein.

In one example, the methods of the present disclosure include the step of determining a level of SM(d41:3) and at least one further lipid biomarker described herein.

In one example, the methods of the present disclosure include the step of determining a level of SM(d42:1) and at least one further lipid biomarker described herein.

In one example, the methods of the present disclosure include the step of determining a level of SM(d42:4) and at least one further lipid biomarker described herein.

In one example, the methods of the present disclosure include the step of determining a level of SM(d44:4) and at least one further lipid biomarker described herein.

In one example, the methods of the present disclosure include the step of determining a level of TG(52:3e) and at least one further lipid biomarker described herein.

In one example, the methods of the present disclosure include the step of determining a level of TG(53:4) and at least one further lipid biomarker described herein.

In one example, the methods of the present disclosure include the step of determining a level of TG(54:3) and at least one further lipid biomarker described herein.

In one example, the methods of the present disclosure include the step of determining a level of TG(54:4) and at least one further lipid biomarker described herein.

In one example, the methods of the present disclosure include the step of determining a level of TG(54:5) and at least one further lipid biomarker described herein.

In one example, the methods of the present disclosure include the step of determining a level of TG(54:6) and at least one further lipid biomarker described herein.

In one example, the methods of the present disclosure include the step of determining a level of TG(56:1) and at least one further lipid biomarker described herein.

In one example, the methods of the present disclosure include the step of determining a level of TG(57:1) and at least one further lipid biomarker described herein.

In one example, the methods of the present disclosure include the step of determining a level of TG(58:1) and at least one further lipid biomarker described herein.

In one example, the methods of the present disclosure include the step of determining a level of TG(58:2) and at least one further lipid biomarker described herein.

In one example, the methods of the present disclosure include the step of determining a level of TG(58:3) and at least one further lipid biomarker described herein.

In one example, the methods of the present disclosure include the step of determining a level of TG(59:2) and at least one further lipid biomarker described herein.

In one example, the methods of the present disclosure include the step of determining a level of TG(60:1) and at least one further lipid biomarker described herein.

In one example, the methods of the present disclosure include the step of determining a level of TG(62:2) and at least one further lipid biomarker described herein.

Any of the methods disclosed herein may not include measuring any other biomarker. Thus, the methods disclosed herein may comprise excluding from analysis any other biomarker.

In particular examples, the methods disclosed herein do not include measuring a level of PC(32:1), PC(38:5) and/or TG(54:4). To this end, the one or more lipid biomarkers may not include PC(32:1), PC(38:5) and/or TG(54:4), or a fragment, variant or derivative thereof.

In some examples, the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(34:1), PC(35:4), PC(36:2), PC(36:3), PC(37:4), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:5), TG(54:6), TG(56:1), TG(57:1), TG(58:1), TG(58:2), TG(58:3), TG(59:2), TG(60:1) and TG(62:2), or a fragment, variant or derivative thereof.

In some examples, the one or more lipid biomarkers may not include Cer(d36:1), LPC(16:0e), PC(32:1), PC(34:1), PC(36:2), PC(36:3), PC(38:5), PE(38:3p), PE(38:4), PE(O-38:5), SM(d36:1), SM(d42:1), TG(52:3e), TG(53:4), TG(54:3), TG(54:4), TG(54:5) and/or TG(54:6), or a fragment, variant or derivative thereof.

In some examples, the one or more lipid biomarkers are selected from the group consisting of Cer(d42:0), Cer(d42:1), DG(34:2), Hex2Cer(d34:1), Hex2Cer(d42:2), LPC(14:0), LPC(17:1), LPC(18:3), LPE(22:6), LPI(20:4), PC(35:4), PC(37:4), PE(34:1), PE(34:2p), PE(36:4), PE(40:5p), PE(40:6e), PI(32:1), PI(34:1), PI(36:1), PI(38:6), PS(38:4), PS(40:6), PS(40:7), SM(d32:2), SM(d33:1), SM(d35:1), SM(d36:2), SM(d38:4), SM(d40:3), SM(d41:2), SM(d41:3), SM(d42:4), SM(d44:4), TG(56:1), TG(57:1), TG(58:1), TG(58:2), TG(58:3), TG(59:2), TG(60:1) and TG(62:2)), or a fragment, variant or derivative thereof.

### Determining the Level of Lipid Biomarkers

It will be understood by the person skilled in the art that the level of expression, abundance or concentration of the one or more lipid biomarkers may be determined by any means known in the art. The terms “determining”, “measuring”, “evaluating”, “assessing”, “quantifying”, “calculating” and “assaying” are used interchangeably herein and may include any form of measurement known in the art, such as those described hereinafter. Such determining may include detecting the presence or absence of one or more of the lipid biomarkers and/or determining a concentration level thereof in the biological sample obtained from the subject.

Suitable means for determining the level of concentration or expression of the one or more lipid biomarkers include, but are not limited to, nuclear magnetic resonance (NMR) spectrometry, surface plasmon resonance (SPR), chromatographic techniques, mass spectrometry, biosensors and any combination of these techniques.

In certain examples, the level of concentration or expression of the one or more lipid biomarkers is measured by mass spectrometry. Mass spectrometry (MS) is an analytical technique that measures the mass-to-charge (m/z) ratio of charged particles. It is primarily used for determining the elemental composition of a sample or molecules, and for elucidating the chemical structures of molecules, such as peptides, lipids and other chemical compounds. MS works by ionizing chemical compounds to generate charged molecules or molecule fragments and measuring their mass-to-charge ratios. MS instruments typically consist of three modules (1) an ion source, which can convert gas phase sample molecules into ions (or, in the case of electrospray ionization, move ions that exist in solution into the gas phase) (2) a mass analyser, which sorts the ions by their masses by applying electromagnetic fields and (3) a detector, which measures the value of an indicator quantity and thus provides data for calculating the abundances of each ion present.

Suitable mass spectrometry methods to be used with the present disclosure include but are not limited to, one or more of electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), tandem liquid chromatography-mass spectrometry (LC-MS/MS) mass spectrometry, desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)n, atmospheric pressure photoionization mass spectrometry (APPI-MS), APPI-MS/MS, and APPI-(MS)n, quadrupole mass spectrometry.

Fourier transform mass spectrometry (FTMS), and ion trap mass spectrometry, where n is an integer greater than zero. In particular examples, the concentration or expression level of the one or more lipid biomarkers is determined at least in part by using liquid chromatography-mass spectrometry (LC-MS).

As noted above. MS ionizes lipids and sorts ions based on their mass-to-charge ratio. It has been widely used to characterize lipids, especially with the development of soft ionization techniques such as electrospray ionization (ESI) and matrix-assisted laser desorption ionization (MALDI). Lipid extraction is usually the first step for lipid analysis, and separates the lipidic components (organic phase) from other components such as proteins and nucleic acids (aqueous phase). Some examples, however, utilise monophasic lipid extraction.

Extraction methods typically include the application of a mixture of methanol, chloroform and water for phase separation. However, shotgun lipidomic methods have also been developed which omit the chromatographic separation and sample processing described above, and analyzes all lipid classes together, and instead using ionization additives to assist in distinguishing between particular lipids. The present MS method may involve lipid digestion, fragmentation or denaturation followed by LC-MS or LC-MS/MS (tandem MS) to derive mass-to-charge ratios for specific lipid headgroups and/or acyl chains that make up the lipid biomarkers described herein.

Suitably, the one or more lipid biomarkers or one or more fragments thereof are subsequently subjected to quantitative mass spectrometry including without limitation, selected reaction monitoring mass spectrometry (SRM), high resolution data independent analyses (SWATH), multiple reaction monitoring (MRM) and/or MSI based quantitation.

In certain examples, an MRM assay is used which employs specific lipids and their fragments (transitions) as discriminators of individual lipid biomarkers.

In particular examples, the present MS method is performed in positive and/or negative ion modes. In general, lipids can form small cation adducts when in the positive-ion mode, due to the ionization process. The formation of cation adducts of lipid molecular species resulted from the affinity of the cations with the dipole that is present in the lipid species depends on the availability of the small cations. By way of example, such adducts can include H⁺, NH4⁺, Li⁺, Na⁺, K⁺, and (—H2O+H)⁺. In the negative-ion mode, lipid species in the deprotonated form or with an anionic adduct are displayed depending on whether the lipid molecule species carry a net ionic bond. For example, PE, PI, PS, PA, and PG, are all of acidic lipid classes (i.e., an ionic bond is present), and thus, may be detected as deprotonated ions. Some lipids are of a polar lipid class without an ionizable bond or PC and SM are strong zwitterionic lipid classes, all of which can form as anionic adducts with small anion(s) (e.g., Cl⁻, CH₃COO⁻, and HCOO⁻) depending on the concentrations present and their affinities with these lipid species.

In some examples, the one or more lipid biomarkers described herein include an ion mode as set out in Tables 8 to 26. Suitably, the ion mode is specific to that method of MS described in the respective Examples.

In other examples, the one or more lipid biomarkers described herein have a retention time or elution time of that or about that as set out in Tables 8 to 26. Suitably, the retention time is determined as per that method of MS described in the respective Examples.

In particular examples, the one or more lipid biomarkers described herein have an accurate mass, neutral mass or mass-to-charge ratio (m/z) of that or about that as set out in the isomer table above or in Tables 8 to 23. Suitably, the neutral mass is determined as per that method of MS described in the respective Examples.

In some applications, various ionization techniques can be coupled to the mass spectrometers provided herein to generate the desired information. Non-limiting exemplary ionization techniques that can be used with the present disclosure include but are not limited to Matrix Assisted Laser Desorption Ionization (MALDI), Desorption Electrospray Ionization (DESI), Direct Assisted Real Time (DART), Surface Assisted Laser Desorption Ionization (SALDI), or Electrospray Ionization (ESI).

In some applications, HPLC and UHPLC can be coupled to a mass spectrometer so that a number of other lipid separation techniques can be performed prior to mass spectrometric analysis. Some exemplary separation techniques which can be used for separation of the desired analyte (e.g., lipid) from the matrix background include but are not limited to Reverse Phase Liquid Chromatography (RP-LC) of lipids, offline Liquid Chromatography (LC) prior to MALDI, 1 dimensional gel separation, 2-dimensional gel separation, Strong Cation Exchange (SCX) chromatography, Strong Anion Exchange (SAX) chromatography, Weak Cation Exchange (WCX), and Weak Anion Exchange (WAX). One or more of the above techniques can be used prior to mass spectrometric analysis.

In some examples, the expression or concentration of a lipid biomarker will be higher in a subject compared to a reference value determined from controls, however for certain lipid biomarkers, expression or concentration of that biomarker is decreased relative to a reference value from controls.

Suitably, an increased level of expression or concentration of a first subset of the one or more lipid biomarkers indicates or correlates with the subject having a breast cancer; and/or a decreased level of expression or concentration of a second subset the one or more lipid biomarkers (e.g., not present in the first subset of the one or more lipid biomarkers) indicates or correlates with the subject having a breast cancer. In other examples, a decreased level of concentration or expression of a first subset of the one or more lipid biomarkers indicates or correlates with the subject not having a breast cancer; and/or an increased level of expression or concentration of a second subset of the one or more lipid biomarkers (e.g., not present in the first subset of the one or more lipid biomarkers) indicates or correlates with the subject not having a breast cancer.

As will be understood by the skilled person, the level or expression level of any one of the lipid biomarkers described herein may be relatively (i) higher, increased or greater: or (ii) lower, decreased or reduced when compared to an expression level in a control or reference sample, or to a threshold expression level. In various examples, an expression level may be classified as higher, increased or greater if it exceeds a mean and/or median expression level of a reference population. In some examples, an expression level may be classified as lower, decreased or reduced if it is less than the mean and/or median expression level of the reference population. In this regard, a reference population may be a group of subjects who have breast cancer. Alternatively, a reference population may be a group of subjects who are known to be free of cancer.

Terms such as “higher”, “increased” and “greater” as used herein refer to an elevated amount or level of a lipid biomarker, such as in a biological sample, when compared to a control or reference level or amount. The concentration or expression level of the lipid biomarker may be relative or absolute (i.e., relatively or absolutely higher, increased or greater). In some examples, the level of a lipid biomarker is higher, increased or greater if its level of concentration or expression is more than about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400% or at least about 500% above the level of concentration or expression of the lipid biomarker in a control or reference level or amount.

The terms, “lower”, “reduced” and “decreased”, as used herein refer to a lower amount or level of a lipid biomarker, such as in a biological sample, when compared to a control or reference level or amount. The concentration or expression level of the lipid biomarker may be relative or absolute (i.e., relatively or absolutely lower, reduced or decreased). In some examples, the concentration or expression of a lipid biomarker is lower, reduced or decreased if its level of concentration or expression is less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level or amount of concentration or expression of the lipid biomarker in a control or reference level or amount.

The term “control sample” typically refers to a biological sample from a (healthy) non-diseased individual not having cancer or, more particularly, not having breast cancer. In some examples, the control sample may be from a subject known to be free of cancer, or more particularly, free of breast cancer. Alternatively, the control sample may be from a subject in remission from cancer.

The control sample may be a pooled, average or an individual sample. An internal control is a marker from the same biological sample being tested.

In some examples, a reference level or amount is determined from measurements of the biomarkers in corresponding panel of biomarkers from a population of healthy individuals. The term “healthy individual” as used herein refers to a person or populations of persons who are known not to have breast cancer. In some examples, the control or reference level is determined from measurements of the corresponding biomarkers in a “typical population”. Preferably, a “typical population” will exhibit a spectrum of breast cancer at different stages of disease progression. It is particularly preferred that a “typical population” exhibits the expression characteristics of a cohort of subjects, such as female subjects, as described herein.

In another example, a reference level or amount may be derived from an established data set including one or more of:
1. a data set comprising measurements of the lipid biomarkers for a subject or a population of subjects known to have breast cancer:
2. a data set comprising measurements of the lipid biomarkers for the subject being tested wherein said measurements have been made previously, such as, for example, when the subject was known to be healthy (i.e., free of breast cancer); and/or
3. a data set comprising measurements of the lipid biomarkers for a healthy individual or a population of healthy individuals.

In certain examples, a data set comprising measurements of the lipid biomarkers may be obtained from a population of subjects known to have breast cancer, a healthy individual or a population of healthy individuals in a fasted state, a non-fasted state or a combination thereof.

As used herein, a concentration or expression level may be an absolute or relative amount of an expressed lipid. Accordingly, in some examples, the concentration or expression level of any one of the one or more lipid biomarkers is compared to a control level of concentration or expression, such as the level of lipid concentration or expression of one or a plurality of “housekeeping” lipids or molecules in the biological sample of the subject.

In further examples, the concentration or expression level of any one of the one or more lipid biomarkers is compared to a threshold level of concentration or expression, such as a level of lipid biomarker concentration or expression in a biological sample from a control subject not having breast cancer and/or an average or median level of lipid biomarker concentration or expression in biological samples derived from a population of breast cancer patients. A threshold level of concentration or expression is generally a quantified level of concentration or expression of a lipid biomarker. Typically, a concentration level or an expression level of a lipid biomarker in a sample that exceeds or falls below the threshold level of concentration or expression is predictive of a particular disease state or outcome, such as the presence or absence of breast cancer. The nature and numerical value (if any) of the threshold level of concentration or expression will typically vary based on the method chosen to determine the concentration or expression of the one or more lipid biomarkers used in determining, for example, a breast cancer diagnosis in the subject.

A person of skill in the art would be capable of determining the threshold level of any one of the one or more lipid biomarkers in a sample that may be used in determining, for example, determining the presence or absence of breast cancer in the relevant subject, using any method of measuring lipid biomarker concentration, abundance or expression known in the art, such as those described herein. In various examples, the threshold level is a mean and/or median concentration or expression level (median or absolute) of the lipid biomarker in a reference population that, for example, have or do not have breast cancer. Additionally, the concept of a threshold level of concentration or expression should not be limited to a single value or result. In this regard, a threshold level of concentration or expression may encompass multiple threshold concentration or expression levels that could signify, for example, a high, medium, or low probability of, for example, the subject having breast cancer.

In view of the foregoing, any of the methods disclosed herein may comprise a step of establishing a reference level or threshold level of concentration or expression of the one or more lipid biomarkers.

Suitably, the predictive accuracy of the methods described herein, as determined by an ROC AUC value, is at least about 0.65 (e.g., at least about 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or any range therein). More particularly, the predictive accuracy of the methods described is suitably at least about 0.70. Even more particularly, the predictive accuracy of the methods described is suitably at least about 0.75. Yet even more particularly, the predictive accuracy of the methods described is suitably at least about 0.80. Still even more particularly, the predictive accuracy of the methods described is suitably at least about 0.85. Yet still even more particularly, the predictive accuracy of the methods described is suitably at least about 0.90.

### Calculating Risk or Diagnostic Scores

For the methods described herein, determining the presence or absence of a breast cancer in a subject may include the step of calculating a risk score or a diagnostic score.

The term “risk score” or “disease risk score” refers to value calculated with one or more feature values or scores that indicates an undesirable physiological state of the patient, such as the presence of cancer. The term “risk score” in certain instances refers to a numerical representation of the current degree of the risk or probability a patient is at for having a particular disease or condition.

A risk score may be calculated using the concentration or expression levels or expression signature of the one or more lipid biomarkers, such as in a panel (e.g., 2, 3, 4, 5 etc or more) of the diagnostic lipid biomarkers, inclusive of those hereinbefore described. To this end, the methods described herein include the step of obtaining a risk score for a lipid biomarker combination hereinbefore described or set forth in any of Examples 1 and 2 (e.g., Tables 8 to 23). A concentration or expression signature of a lipid may be determined using the normalized level of concentration or expression of the lipid biomarker in a sample, and an independent diagnostic value of the lipid biomarker based on the correlation of the concentration or expression of the lipid biomarker with disease presence or absence. Any method of determining a concentration or expression signature for a lipid biomarker known in the art may be utilised. After determining the concentration or expression levels or expression signatures of individual lipid biomarkers, such as in a panel of two or more of the lipid biomarkers described herein, a risk score may be calculated by combining the concentration or expression levels and/or the expression signatures of each lipid biomarker in a panel thereof. Methods of calculating a risk score may be as described in the Examples.

In particular examples, the risk score is calculated at least in part by logistic regression. By way of example, a linear combination of the concentration or expression levels of the one or more lipid biomarkers with various coefficients determined through prior training may be generated and subsequently used to estimate the log odds of cancer. The log odds can then be converted into a probability of a subject having breast cancer via logistic regression. In other examples, the risk score is calculated at least in part by partial least squares discriminant analysis.

Accordingly, a risk score for a patient may be calculated according to the below formula:

$$Probability of cancer = {e}^{I + \sum ci * Li} / ( 1 + {e}^{I + \sum ci * Li} )$$

Wherein the intercept I and coefficients ci are the specific logistic regression model parameters calculated in advance based on the training data, and the values Li represent the normalised lipid abundances measured for the respective patient sample for each lipid in the panel.

A calculated risk score of the disclosure may be used to determine the likelihood of the presence or absence of a breast cancer in a subject. In general, a calculated risk score may be compared to a reference risk score. In certain examples, if (i) the risk score is equal to or higher than the reference risk score, the subject has a breast cancer, and (ii) the risk score is lower than the reference risk score, the subject does not have a breast cancer. It is envisaged that a subject's diagnosis and/or risk score can be utilised to determine whether said subject should be treated with an anti-cancer agent. Accordingly, in other examples, if (i) the risk score is equal to or higher than the reference risk score, the subject is to be administered an anti-cancer treatment, and (ii) the risk score is lower than the reference risk score, the subject is not to be administered an anti-cancer treatment.

In some examples, the risk score is compared to a threshold risk score, such as a median or average risk score, to determine the diagnosis of breast cancer in a subject. If the risk score is equal to or higher than the threshold risk score, the subject suitably has breast cancer. Alternatively, if the risk score is lower than the threshold risk score, the subject suitably does not have breast cancer. A threshold risk score may be the respective median or average of the risk scores calculated for each subject in a population of subjects with breast cancer and/or the respective median or average of the risk scores calculated for each subject in a population of subjects without breast cancer, such as those subjects described in Example 1.

### Kits

The present disclosure also contemplates kits for the detection of lipid biomarkers that may be suitable for use in the methods described herein.

In one broad form, the present disclosure provides a kit for determining the presence or absence of a breast cancer in a subject, the kit comprising one or more reagents for determining a level of one or more lipid biomarkers in a biological sample obtained from the subject, wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

Any agent or probe capable of binding specifically to a biomarker gene product will be useful, such as a set of primers, a labelled nucleic acid probe, an aptamer, an antibody and/or an antibody fragment. Other components of the kits will typically include labels, secondary antibodies, inhibitors, co-factors and control lipid product preparations to allow the user to quantitate concentration or expression levels and/or to assess whether the measurement has worked correctly. Biosensors, including optical (e.g., SPR-based sensors, interferometry-based sensors, waveguide-based sensors), electrochemical and mechanical biosensors are particularly suitable assays that can be carried out easily by the skilled person using kit components.

In some examples, the kit may comprise a substrate, such as a microtitre plate, on which is immobilised capture probes or antibodies corresponding to the lipid biomarkers being measured.

In some examples, the kit comprises beads on which is immobilised capture probes or antibodies corresponding to the lipid biomarkers being measured.

Optionally, the kit further comprises means for the detection of the binding of a probe, such as an antibody, to a lipid biomarker. Such means include a reporter molecule such as, for example, an enzyme (such as horseradish peroxidase or alkaline phosphatase), a dye, a radionucleotide, a luminescent group, a chemiluminescent group, a fluorescent group, biotin or a colloidal particle, such as colloidal gold or selenium. Suitably, such a reporter molecule is directly linked to the antibody.

In one example, a kit may additionally comprise a reference sample. Suitably, a reference sample comprises a reference lipid that is detected by an antibody and/or may be labelled or modified so as to be distinguished from native lipid. Preferably, the reference lipid is of known concentration.

Such a reference lipid is of particular use as a standard. Accordingly, various known concentrations of such a reference lipid may be detected using a diagnostic assay described herein.

Instructions supplied in the kits of the present disclosure are typically written instructions on a label or package insert (e.g., a paper sheet included in the kit), but machine-readable instructions (e.g., instructions carried on a magnetic or optical storage disk) are also acceptable. The instructions relating to the use of the reagents described herein, generally include information as to determining a concentration or expression level of the one or more lipid biomarkers and guidance regarding dosage, dosing schedule, and route of administration for an indicated treatment. The kit may further comprise a description of selecting an individual having breast cancer and thereby suitable for treatment.

In particular examples, the reference data is on a computer-readable medium (e.g., software embodying or utilized by any one or more of the methodologies or functions described herein). The computer-readable medium can be included on a storage device, such as a computer memory (e.g., hard disk drives or solid state drives) and may comprise computer readable code components that when selectively executed by a processor implements one or more aspects of the present disclosure.

### Systems

The present disclosure also contemplates systems for the detection of lipid biomarkers that may be suitable for use in the methods described herein.

In one broad form, the present disclosure provides a system for determining the presence or absence of a breast cancer in a subject, the system comprising:
a mass spectrometry unit configured for determining a level of one or more lipid biomarkers in a biological sample obtained from the subject, wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof; and
a processing unit configured for using or analysing the level of the one or more lipid biomarkers to determine the presence or absence of the breast cancer in the subject.

It is noted that the step of determining a concentration level or an expression level may be performed by the mass spectrometry units and/or may be performed at least in part by a pre-processing unit. That pre-processing unit may be the same as or different to the processing unit performing the steps of analysing the concentration or expression level. For example, the pre-processing unit may receive data from the mass spectrometry unit indicative of a number of fragments (e.g., lipid headgroup and/or acyl chain) of the lipid biomarkers for respective mass values. This data may also be representative of the retention time of particular fragments. The pre-processing unit may then process this data to determine lipid biomarkers as combinations of fragments to thereby calculate the corresponding concentration or expression levels.

Suitably, the mass spectrometry unit and the processing unit are that described herein.

### Computer-Implemented Methods

It is envisaged that one or more steps of the methods described herein may be automated or implemented by a computer in the sense that the disclosed methods are implemented as software code that is stored on a non-volatile data storage medium. The computer executes the software code, which causes the computer to perform the methods disclosed herein.

By way of example, comparing a concentration level or an expression level of the one or more lipid biomarker with, for example, a reference or threshold level or value may be carried out by a computer executing software code describing the comparing step. Thus, the comparison may be carried out by a computer or computing device, such as by a processing unit. The value of the determined or detected amount of the one or more lipid biomarkers in the sample from the subject and the reference amount can be, for example, compared to each other and said comparison can be automatically carried out by a computer program executing an algorithm for the comparison. Additionally, the calculation of a risk or diagnostic score and/or its comparison to a reference risk or diagnostic score can be automatically carried out by a computer program executing an algorithm for the comparison. Suitably, such algorithms may be trained on one or more case and/or control samples. In some examples, a processor may utilize the concentration or expression level data and/or a risk or diagnostic score to calculate a likelihood of the subject in question having a breast cancer.

The computer program carrying out the said evaluation will suitably provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provide the desired assessment in a suitable output format.

In some examples, the methods of the disclosure include one or more of the broad steps of:
(i) optionally performing a measurement of the concentration or expression level of the one or more lipid biomarkers described herein:
(ii) inputting or receiving the values from (i) into a processing system that is configured to determine the presence or absence of a breast cancer in a subject:
(iii) optionally calculating a risk or diagnostic score from the level or expression level of the one or more lipid biomarkers by the processing system:
(iv) comparing the concentration or expression level and/or the risk or diagnostic score obtained in step (iii) with a threshold or reference value by the processing system; and
(v) determining the presence or absence of the breast cancer in the subject.

The methods of the present disclosure suitably permit integration into existing or newly developed pathology architecture or platform systems. For example, the present disclosure contemplates a method of allowing a user to determine the status (e.g., the presence or absence of a breast cancer) of a subject, the method including the steps of:
(a) receiving data in the form of concentration or expression levels of one or more lipid biomarkers for a test sample, wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof;
(b) optionally processing the subject data, such as with a processing unit or system, via univariate and/or multivariate analysis and/or machine learning algorithms (e.g., LASSO-penalised multivariate Cox regression, logistic regression, partial least squares discriminant analysis, random forest, decision tree, gradient boosting) to provide a risk or diagnostic score:
(c) determining a status of the subject in accordance with the results of the concentration or expression levels and/or the risk or diagnostic score in comparison with predetermined or reference concentration or expression levels and/or risk or diagnostic score values, such as with a processing unit or system; and
(d) transferring an indication of the status of the subject to the user.

In some examples, the above method further includes the step of producing or generating the concentration level or expression level data by determining a concentration level or an expression level of one or more lipid biomarkers in the biological sample obtained from the subject, wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof, such as by using one or more of those methodologies described herein.

In one example, the method additionally includes:
(a) having a user determine the data using a remote end station; and
(b) transferring the data from the end station to a base station via a communications network.

The base station can include first and second processing systems, in which case the method can include:
(a) transferring the data to the first processing system:
(b) causing the first processing system to perform a univariate or multivariate analysis function to generate the risk or diagnostic score.

The method may also include:
(a) transferring the results of the univariate or multivariate analysis function and/or the determined concentration or expression levels of the lipid biomarkers to the second processing system; and
(b) causing the second processing system to determine the status of the subject.

The second processing system may be coupled to a database adapted to store predetermined data and/or the univariate or multivariate analysis function, such that the computer-implemented method may include:
(a) querying the database to obtain at least selected predetermined data or access to the univariate or multivariate analysis function from the database; and
(b) comparing the selected predetermined data to the subject data or generating a predicted probability index.

The second processing system can be coupled to a database, the method including storing the data in the database, such as by way of a memory unit.

The reference concentration or expression level data comprises a level or a level of concentration or expression determined for the one or more lipid biomarkers within a biological sample selected from the group consisting of:
(i) a biological sample from a normal or healthy subject, such as normal or healthy subject without breast cancer:
(ii) a biological sample from a subject previously diagnosed or determined as having a breast cancer:
(iii) an extract of any one of (i) to (ii):
(iv) a data set comprising levels of concentration or expression for the lipid biomarkers within a normal or healthy individual or a population of normal or healthy individuals:
(vi) a data set comprising levels of concentration or expression for the lipid biomarkers in an individual or a population of individuals having breast cancer; and
(vii) a data set comprising levels of concentration or expression for the lipid biomarkers in the subject being tested wherein the levels of concentration or expression are determined for a sample having been taken at an earlier time point when the subject was known to not have breast cancer.
   Obtaining a Sample from a Subject

The methods disclosed herein may further include the initial or earlier step of providing or collecting a biological sample from the subject that suitably contains lipid micro-vesicles or extracellular vesicles. Such a sample may be obtained by freshly collecting a sample, or may be obtained from a previously collected and stored sample. By way of example, a sample may be obtained from a previously collected and stored (e.g., refrigerated or frozen) blood or serum sample. Suitably, a sample is obtained by freshly collecting a sample from the subject. Alternatively, a sample can be obtained from a previously collected and stored sample from the subject.

In particular examples, the subject has fasted prior to collection or is in a fasted state at the time of collection of the biological sample for further testing by the methods provided herein. As used herein, the term “fasted” refers to the condition of not having consumed food or beverage during the period between from at least about 3 hours to about 12 hours (e.g., 3, 4, 5, 6, 7, 8, 9, 10, 11, or 12 hours and any range therein) prior to providing a biological sample for testing.

In alternative examples, the subject has not fasted prior to collection or is in a non-fasted (or “fed”) state at the time of collection of the biological sample for further testing by the methods provided herein. The term “non-fasted” as generally used herein refers to the condition of having consumed food and/or beverage within at least about 3 hours to about 12 hours (e.g., 3, 4, 5, 6, 7, 8, 9, 10, 11, or 12 hours and any range therein) of providing a biological sample for testing.

The methods of the present disclosure can be performed on various biological samples. As used herein, the term “biological sample” is suitably a sample obtained from a subject. For example, the biological sample can be a bodily fluid of the subject. Suitable biological samples typically comprise a concentration of lipid micro-vesicles or extracellular vesicles. A suitable biological sample may also comprise circulating lipid micro-vesicles or extracellular vesicles. Circulating lipid micro-vesicles or extracellular vesicles may be found in a bodily fluid (e.g., blood, plasma, serum, urine, vomit, tears, sputum etc.) or other excrement (e.g., faeces). In certain examples, the biological sample is or comprises blood, plasma or serum. In particular examples, the biological sample is blood. In other examples, the biological sample is plasma. In various examples, the biological sample is serum.

Suitably, the biological sample has been enriched for extracellular vesicles and/or a lipid content thereof. In particular examples, the biological sample is or comprises a population of extracellular vesicles or exosomes. For some examples, the biological sample is or comprises a population of extracellular vesicles or exosomes obtained from a patient diagnosed with or suspected of having breast cancer. According to certain examples, the biological sample is purified or partially purified to isolate extracellular vesicles, including cancer-derived extracellular vesicles, therefrom according to the present disclosure. For example, a serum sample may be purified to remove cells. In certain examples, other components (e.g. debris, albumin) originally within the biological sample are removed or partially removed from the biological sample before performing the methods of the present disclosure. In an example, the biological sample is substantially free of cells.

The sample includes extracts, derivatives, fractions or suspensions of an original biological sample obtained from a subject disclosed herein.

Suitably, the biological samples described herein comprise a concentration of extracellular vesicles, such as cancer-derived extracellular vesicles or exosomes. A suitable biological sample may also comprise circulating extracellular vesicles. Circulating extracellular vesicles may be found in a bodily fluid (e.g., blood, plasma, serum, urine, vomit, tears, sputum etc.) or other excrement (e.g., faeces). In particular examples, the biological sample is or comprises an extracellular vesicle sample. In other examples, the biological sample is or comprises an exosome sample or exosomal sample. To this end, aspects of the present disclosure suitably comprise determining a level of EV or exosomal expression or concentration of the lipid biomarkers hereinbefore described in the biological sample obtained from the subject. Put another way, the present methods may include determining or measuring the expression levels of two or more EV- or exosome-associated biomarkers as provided herein to the exclusion of protein biomarkers not associated with EVs or exosomes (e.g., biomarkers associated with free protein or free RNA in the biological sample).

Accordingly, determining a level of EV expression of a biomarker includes determining an expression level of said biomarker associated with an EV or population of EVs. Similarly, determining a level of exosomal expression of a biomarker includes determining an expression level of said biomarker associated with an exosome or population of exosomes. The terms “EV-associated”, “exosome-associated” and the like refer to a biomolecule, such as a lipid biomarker, located within/inside or on the surface of an EV or exosome respectively. To this end, EVs and exosomes may carry or contain cellular biomolecules within their lipid membrane. Since EVs and exosomes are typically formed from membranes of a cell, they may also carry molecules on their surface. Additionally, since some biomolecules bind those on the surface of EVs and exosomes, these biomolecules may also indirectly be considered “EV-associated” or “exosome-associated”. Moreover, the present disclosure envisions detecting an EV- or exosome-associated biomarker even after such a biomarker has been separated from the EV or exosome with which it may have been originally associated.

The biological sample may be subject to any suitable pre-treatment steps before measurement of the level of the one or more lipid biomarkers is performed, in order to improve the accuracy and/or efficiency of the measurement. Such pre-treatment steps may include extraction, centrifugation (e.g., ultracentrifugation), lyophilization, fractionation, separation (e.g., using column or gel chromatography), concentration or evaporation. In some instances, this treatment can include one or more extractions with solutions comprising any suitable solvent or combinations of solvents, such as, but not limited to acetonitrile, water, chloroform, methanol, butylated hydroxytoluene, trichloroacetic acid, toluene, hexane, benzene, or combinations thereof. In some examples, the biological sample may undergo one or more treatment steps so as to isolate, concentrate or enrich for extracellular vesicles and/or a lipid content thereof. Suitably, the biological sample is an extracellular vesicle sample or is a sample that has been enriched for extracellular vesicles.

As used herein, the term “extracellular vesicle” or “EV” refers to a cell-derived vesicle comprising a membrane that encapsulates an interior space. Extracellular vesicles include all membrane-bound vesicles, whose diameter is typically smaller than the diameter of the cell from which they are derived. More particularly, extracellular vesicles can include small extracellular vesicles (50-200 nm), microvesicles (0.2-1 μm), exomeres and exosomes (30-150 nm), and oncosomes (1 μm up to 10 μm). According to various examples, the extracellular vesicles are or comprise exosomes. In certain examples, the extracellular vesicles have an average diameter of between about 10 nm to about 250 nm (e.g., about 10, 20, 30, 40, 50, 60, 70, 80, 90, 100, 110, 120, 130, 140, 150, 160, 170, 180, 190, 200, 210, 220, 230, 240, 250 nm or any range therein), more particularly between about 50 nm to about 200 nm and even more particularly between about 30 nm to about 150 nm).

In view of the above, the biological sample (e.g., blood, plasma and/or serum) may have been enriched for extracellular vesicles, such as cancer-derived extracellular vesicles or exosomes. In some examples, the biological sample is a purified or partially purified population of extracellular vesicles or exosomes. In other examples, the biological sample is a purified population of exosomes. In further examples, the biological sample is a partially purified population of exosomes. As such, in some examples, the methods described herein include the initial or earlier step of at least partly enriching, concentrating, isolating or purifying a population or fraction of extracellular vesicles or exosomes from the biological sample of the patient in question, such as by those methods described herein.

A population or fraction of extracellular vesicles or exosomes may be enriched, concentrated, isolated or purified from a biological fluid, such as those provided above, so as to facilitate the removal of contaminating proteins, lipoproteins etc. To this end, extracellular vesicles or exosomes may be isolated by any means known in the art, such as, but not limited to, polyethylene glycol precipitation, high MW centrifugal filtration, ultracentrifugation, size-exclusion chromatography, exosome precipitation (e.g., ExoQuick from System Biosciences), tangential flow filtration, affinity-based capture of exosomes (e.g., affinity purification with binding molecules or antibodies to CD63, CD81, CD82, CD9, Alix, annexin, EpCAM, and Rab5), immuno-magnetic bead capture (e.g., EXO-NETR system from INOVIQ) and any combination thereof. In various examples, extracellular vesicles or exosomes are at least partly isolated from the biological sample by ultracentrifugation. In certain examples, extracellular vesicles or exosomes are at least partly isolated from the biological sample by size exclusion chromatography. In other examples, extracellular vesicles or exosomes are at least partly isolated from the biological sample by affinity-based methods, and more particularly immuno-magnetic bead capture.

As used herein, the term “isolate”, “isolating” or “isolation” refers to material, such as extracellular vesicles or exosomes, that has been removed from its natural state or otherwise been subjected to human manipulation. Isolated material may be substantially or essentially free from components that normally accompany it in its natural state, or may be manipulated so as to be in an artificial state together with components that normally accompany it in its natural state. These terms include gross physical separation of the material, inclusive of extracellular vesicles, from their natural environment (e.g. removal/purification from a biological sample obtained from a subject suspected of having breast cancer).

Thus, any of the methods disclosed herein may comprise a step of taking a biological sample from a subject and determining the level of expression, concentration or abundance of the one or more lipid biomarkers in the sample. Alternatively, any of the methods disclosed herein may not comprise a step of taking a biological sample from a subject and determining the level of expression, concentration or abundance of the one or more lipid biomarkers in the sample. Instead, the level of expression, concentration or abundance of the one or more lipid biomarkers in the sample may have been determined previously

So that preferred embodiments of the present disclosure may be fully understood and put into practical effect, reference is made to the following non-limiting examples.

### EXAMPLES

### Example 1. Exploratory Assessment of Lipidomics Biomarkers in Four Breast Cancer Cohorts

The aim of the present Example was to establish prediction models, from lipidomics data, for breast cancer.

Table of abbreviations

| | |
|---|---|
| AUC | Area Under the Curve |
| BMI | Body Mass Index |
| DCIS | Ductal Carcinoma In Situ |
| GB | Gradient Boosting |
| HBV | Hepatitis B Virus |
| HCV | Hepatitis C Virus |
| HDL | High Density Lipoprotein |
| HIV | Human Immunodeficiency Virus |
| IDC | Invasive Ductal Carcinoma |
| ILC | Invasive Lobular Carcinoma |
| IQR | Interquartile Range |
| LC-MS | Liquid Chromatography-Mass Spectrometry |
| LDL | Low Density Lipoprotein |
| ML | Machine Learning |
| NPV | Negative Predictive Value |
| PCA | Principal Components Analysis |
| PPV | Positive Predictive Value |
| ROC | Receiver Operating Characteristics |
| SD | Standard Deviation |

### Study Design

Four sets of plasma samples, obtained with informed consent from adult female patients, were utilised for lipidomics analysis during 2018, 2019, 2020 and 2021. The disease characteristics of the individuals from whom the samples were derived spanned invasive ductal carcinoma (IDC) of various stages, ductal carcinoma in situ (DCIS), invasive lobular carcinoma (ILC) and normal controls, as summarised in Table 1. All individuals were fasted prior to blood draw. It is not known whether any individual is included in more than one-dataset.

TABLE 1
Counts by disease classification across the four datasets

| | | | | | | | | |
|---|---|---|---|---|---|---|---|---|
| | IDC | IDC | IDC | IDC | IDC | | | |
| | Stage | Stage | Stage | Stage | Stage | | | |
| Dataset | I | II | III | IV | III/IV | DCIS | ILC | Control |

| | | | | | | | |
|---|---|---|---|---|---|---|---|
| 1 | 15 | 15 | 11 | 4 | | | 45 |
| 2 | 50 | 50 | | | | | 101 |
| 3 | 50 | 50 | | | | | 101 |
| 4 | | | 52 | 50 | 50 | 50 | 100 |

### Cases

In all four datasets, breast cancer type was clinically and morphologically confirmed; stage of breast cancer was confirmed by biopsy.

Breast cancer patients were excluded according to the following criteria:
Pregnant or breastfeeding.
History of HBV, HCV or HIV
Prior breast cancer treatment
Prior breast cancer surgery

In datasets 2-4, cases satisfied additional criteria namely, the absence of:
Other chronic illness
History of cardiovascular disease or diabetes
Hyperlipidemia or dyslipidemia
Radiation or chemotherapy

### Controls

In all four datasets, normal controls were individuals without a diagnosis of breast cancer, category-matched to cases by age and BMI.

In datasets 2-4, normal controls satisfied additional criteria namely the absence of
Chronic illness
History of cardiovascular disease or diabetes
Hyperlipidemia or dyslipidemia
History of any cancer
Radiation or chemotherapy

### Sample Size Considerations

The current analysis was a post-hoc exploratory analysis of existing data. The four datasets comprising the current analysis were pilot datasets, derived without power calculations in relation to the current analysis.

### Analysis Populations

The following analysis populations were considered for discovery purposes:
1. Dataset 1
2. Dataset 2
3. Dataset 3
4. Dataset 4
5. Dataset 2+Dataset 3+Dataset 4

Test performance calculations involved multiple populations as shown in Table 3.

TABLE 3
Analysis populations

| | |
|---|---|
| Discovery | Test Performance |

| | | | |
|---|---|---|---|
| Dataset 1 | Dataset 2 | Dataset 3 | Dataset 4 |
| Dataset 2 | Dataset 1 | Dataset 3 | Dataset 4 |
| Dataset 3 | Dataset 1 | Dataset 2 | Dataset 4 |
| Dataset 4 | Dataset 1 | Dataset 2 | Dataset 3 |
| Dataset 2 + | Dataset 1 | Dataset 2 + | |
| Dataset 3 + | | Dataset 3 + | |
| Dataset 4 | | Dataset 4 | |
| (Training Set) | | (Validation Set) | |

### General Considerations for Data Analysis

### Multiple Batches

The looser inclusion criteria for Dataset 1 meant that it may include individuals with a diagnosis of, or receiving treatment for, traits such as dyslipidemia, which affect the lipid profile.

While datasets 2 and 3 were similar in design, all four datasets were obtained at different time-points and were subjected to separate laboratory runs. There were, in total six laboratory batches because Datasets 2 and 3 were each analysed in two tranches.

In general, amongst the cases, a spectrum of breast cancer types and stages are considered. For these reasons, in the first instance, the search for predictors was conducted in each dataset, one at a time.

### Multiple Comparisons and Multiplicity

In the interests of statistical power, no adjustment for multiple testing was performed.

### Study Population

### Clinical Characteristics

Tabular summaries of clinical characteristics were made, one dataset at a time, according to the availability of data (Tables 4-7). All tables were stratified by case/control status and included entries for age, BMI, HDL and LDL. In addition, total cholesterol and triglycerides, were tabulated for Datasets 2-4. All participants self-reported as being of white, non-Hispanic origin. Categorical variables were summarised as n (%): continuously symmetrically distributed variables were summarised as Mean (SD): continuously distributed variables with skewed distribution were summarised as Median (IQR).

Non-parametric tests were used to compare the distribution of these variables in cases and controls. A Pearson's Chi-Squared test was applied for categorical variables: a Mann-Witney U Test was applied for continuously distributed variables. The resultant was expressed to three decimal places, or as p<0.001. Missing values for clinical data were not imputed. It was noted that for Dataset 2, batch was confounded with case-control status (p<0.001). This means that after adjustment for batch, the statistical power of this dataset will be reduced.

Differences in lipid levels (cholesterol, HDL and LDL) were observed between cases and controls in cohort 3 and/or 4. Due to the complex inter-relationship between lipids and disease status, a decision was made not to impose a statistical adjustment for any of these measures.

TABLE 4
Demographic characteristics of Dataset 1

| | Statistic | Case | Control | P |
|---|---|---|---|---|
| Count | N | 45 | 45 | NA |
| Disease Stage I | n (%) | 15 (33.3) | 0 (0) | NA |
| Disease Stage II | n (%) | 15 (33.3) | 0 (0) | NA |
| Disease Stage | n (%) | 11 (24.4) | 0 (0) | NA |
| III | | | | |
| Disease Stage | n (%) | 4 (8.9) | 0 (0) | NA |
| IV | | | | |
| Tumor Grade | n (%) | 1 (2.2) | 0 (0) | NA |
| G1 | | | | |
| Tumor Grade | n (%) | 38 (84.4) | 0 (0) | NA |
| G2 | | | | |
| Tumor Grade | n (%) | 5 (11.1) | 0 (0) | NA |
| G3 | | | | |
| Age | Mean (SD) | 58.69 (12.62) | 59.67 (11.25) | 0.728 |
| BMI | Mean (SD) | 29.26 (6.81) | 28.33 (5.55) | 0.561 |
| HDL | Mean (SD) | 1.16 (0.4) | 0.97 (0.27) | 0.346 |
| LDL | Mean (SD) | 2.66 (1.01) | 2.43 (0.92) | 0.907 |

TABLE 5
Demographic characteristics of Dataset 2

| | Statistic | Case | Control | P |
|---|---|---|---|---|
| Count | N | 100 | 101 | NA |
| Batch | n (%) | 63 (63) | 36 (35.6) | <0.001 |
| Disease Stage I | n (%) | 50 (50) | 0 (0) | NA |
| Disease Stage II | n (%) | 50 (50) | 0 (0) | NA |
| Tumor Grade | n (%) | 30 (31.6) | 0 (0) | NA |
| G1 | | | | |
| Tumor Grade | n (%) | 56 (58.9) | 0 (0) | NA |
| G2 | | | | |
| Tumor Grade | n (%) | 8 (8.4) | 0 (0) | NA |
| G3 | | | | |
| Tumor Grade | n (%) | 1 (1.1) | 0 (0) | NA |
| G4 | | | | |
| Age | Mean (SD) | 60.01 (10.64) | 63.23 (12.35) | 0.054 |
| BMI | Mean (SD) | 28.41 (4.35) | 28.12 (4.46) | 0.677 |
| Cholesterol | Mean (SD) | 5.79 (1.18) | 5.5 (1.04) | 0.144 |
| HDL | Mean (SD) | 1.66 (0.48) | 1.52 (0.34) | 0.098 |
| LDL | Mean (SD) | 3.46 (0.93) | 3.3 (1) | 0.431 |
| TGL | Median (IQR) | 1.3 (0.9, 1.7) | 1.3 (1, 1.8) | 0.636 |

TABLE 6
Demographic characteristics of Dataset 3

| | Statistic | Case | Control | P |
|---|---|---|---|---|
| Count | N | 100 | 101 | NA |
| Batch | n (%) | 47 (47) | 46 (45.5) | 0.948 |
| Disease Stage I | n (%) | 50 (50) | 0 (0) | NA |
| Disease Stage II | n (%) | 50 (50) | 0 (0) | NA |
| Tumor Grade | n (%) | 5 (5.1) | 0 (0) | NA |
| G1 | | | | |
| Tumor Grade | n (%) | 76 (77.6) | 0 (0) | NA |
| G2 | | | | |
| Tumor Grade | n (%) | 15 (15.3) | 0 (0) | NA |
| G3 | | | | |
| Age | Mean (SD) | 54.56 (11.39) | 52.77 (10.24) | 0.224 |
| BMI | Mean (SD) | 27.04 (4.79) | 26.76 (4.25) | 0.698 |
| Cholesterol | Mean (SD) | 4.95 (0.76) | 5.19 (0.68) | 0.027 |
| HDL | Mean (SD) | 1.34 (0.36) | 1.39 (0.37) | 0.424 |
| LDL | Mean (SD) | 3.34 (0.81) | 3.51 (0.75) | 0.134 |
| TGL | Median (IQR) | 1.23 (0.84, 1.53) | 1.28 (0.96, 1.69) | 0.282 |

TABLE 7
Demographic characteristics of Dataset 4

| | Statistic | Case | Control | P |
|---|---|---|---|---|
| Count | N | 201 | 202 | NA |
| Disease Stage | n (%) | 52 (25.9) | 0 (0) | NA |
| III | | | | |
| Disease Stage | n (%) | 50 (24.9) | 0 (0) | NA |
| IV | | | | |
| Tumor Grade | n (%) | 5 (3.5) | 0 (0) | NA |
| G1 | | | | |
| Tumor Grade | n (%) | 90 (63.8) | 0 (0) | NA |
| G2 | | | | |
| Tumor Grade | n (%) | 40 (28.4) | 0 (0) | NA |
| G3 | | | | |
| Age | Mean (SD) | 56.7 (11.87) | 55.27 (12.25) | 0.321 |
| BMI | Mean (SD) | 27.29 (4.6) | 27.43 (4.85) | 0.920 |
| Cholesterol | Mean (SD) | 4.96 (0.86) | 5.25 (0.74) | 0.004 |
| HDL | Mean (SD) | 1.39 (0.44) | 1.48 (0.35) | 0.018 |
| LDL | Mean (SD) | 3.22 (0.94) | 3.51 (0.94) | 0.012 |
| TGL | Median (IQR) | 1.26 (0.85, 1.69) | 1.2 (0.89, 1.85) | 0.808 |

### Biomarker Data Analysis

### Introduction

Lipidomics data was generated using liquid chromatography-mass spectrometry (LC-MS). Lipids were identified and quantified using LipidSearch™ Software and Skyline. The process involved peak detection and database matching, and the generation of quality scores for filtering purposes. The data to be statistically analysed comprised normalised lipid concentrations.

### Exploratory Data Analysis

The analyses in this section were conducted one dataset at a time, on cases and controls combined. In addition, a merged data set comprising datasets 2-4 was considered. The latter was labelled ‘234’.

Missing values were not present in the data as such however, zeroes were seen. Lipids with coefficient of variation less than 0.20 in the combined case-control set were excluded from further consideration. The distribution of each lipid was plotted as a histogram, and a global natural log-transformation (ln(1+x)) was applied to improve symmetry.

Principal Components Analysis (PCA) was performed, and results were plotted in two and three dimensions, coloured by case-control status. Extreme outliers, if detected were excluded and the plot redrawn. For analysis populations involving multiple laboratory batches, adjustment for batch was applied while accommodating case-control status, by using Empirical Bayes Methodology (Johnson and Rabinovic, 2007). The PCA plot was re-drawn.

After completing the steps above, five analysis-ready data sets were available, comprising 394 lipids, 386 lipids, 382 lipids, 380 lipids and 373 lipids respectively for data sets 1, 2, 3, 4 and 234.

### Training and Validation Set for Data Set 234

Data set ‘234’ was split into training and validation sets in the ratio 2:1, balancing for cohort, case/control status and age. The training set was used for signature discovery: the validation set was used for test performance calculations. The following signature discovery steps were performed for each dataset individually, and for the 234 Training set: there were five rounds in total.

### Initial Coarse Filter

Each lipid was tested, one at a time for association with case/control status in a logistic model, adjusting for any covariates established in the prior step. Any lipid with p-value >=0.05 was excluded from further consideration.

### Determination of Lipid Clusters

For the purposes of deriving knowledge-based signatures of breast cancer, the remaining lipids were grouped according to their numerical correlation. A similarity matrix was created for the 30 strongest associations arising from each dataset. Hierarchical cluster analysis was applied, and a dendrogram was plotted.

### Machine Learning

Machine learning was conducted by gradient boosting (Friedman 2001; Friedman 2002; Chen et al, 2021). Gradient boosting is an approach to determine a regression function that minimises the expectation of a loss function. It is an iterative method, in which the negative gradient of the loss function is calculated, a regression model is fitted, the gradient descent step size is selected, and the regression function is updated. The gradient is approximated by means of a regression tree, and at each iteration the gradient determines the direction in which the function needs to move, to improve the fit to the data.

The loss function for the current binary endpoints was specified as Bernoulli. A learning rate was introduced to dampen proposed moves and to protect against over-fitting. The minimum number of observations in each terminal node was 10. Two-way interactions were allowed. Random sub-sampling, without replacement, of half of the observations was applied to achieve variance reduction in gradient estimation. There were 100 rounds of gradient boosting per model.

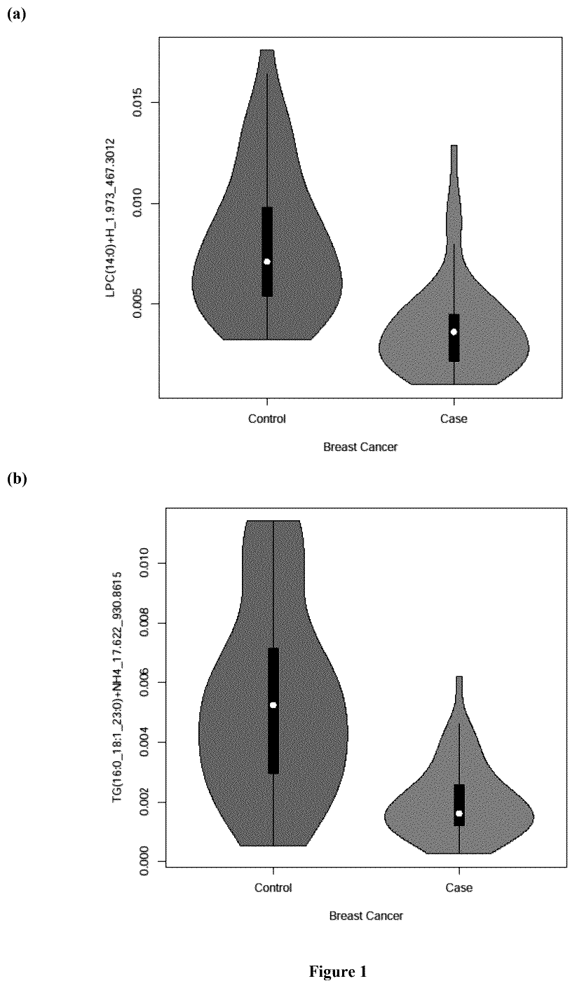

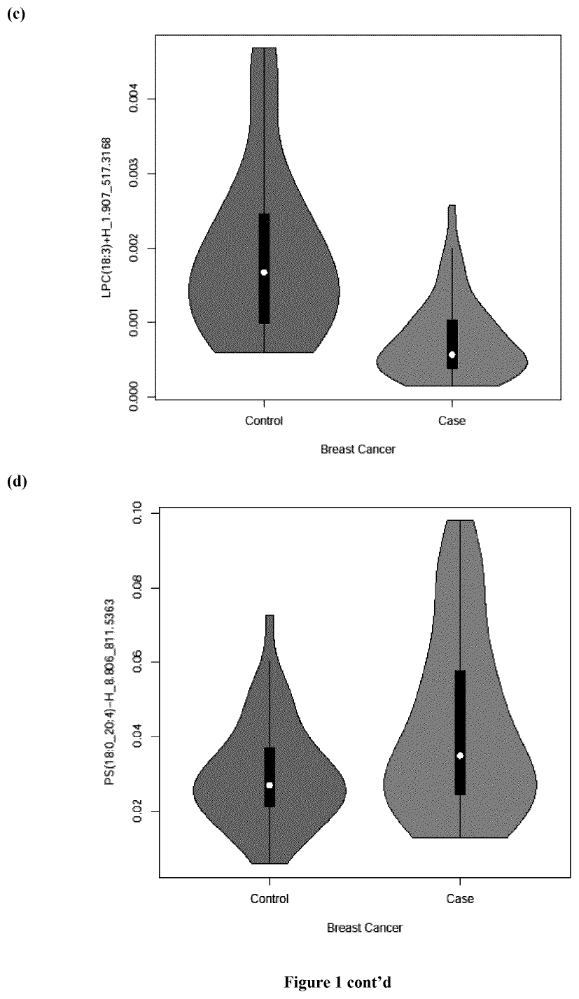

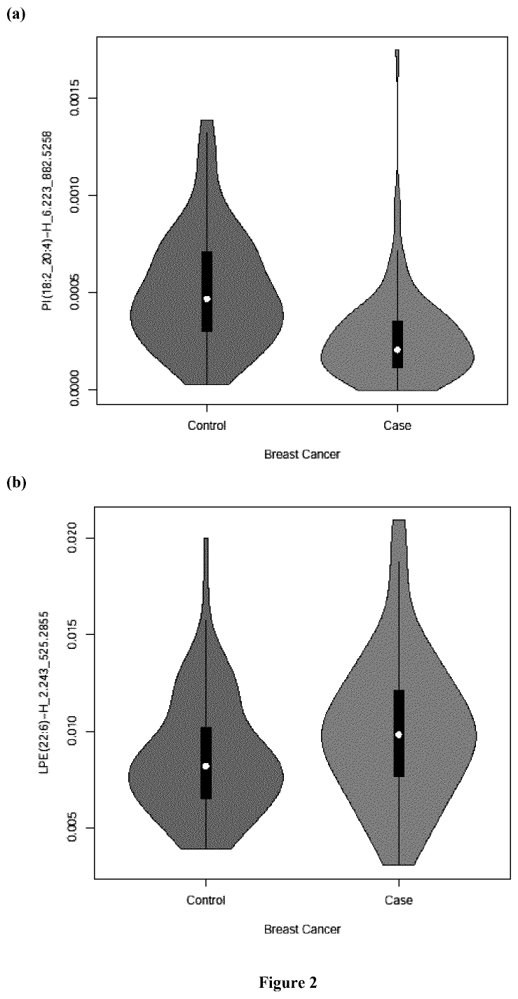

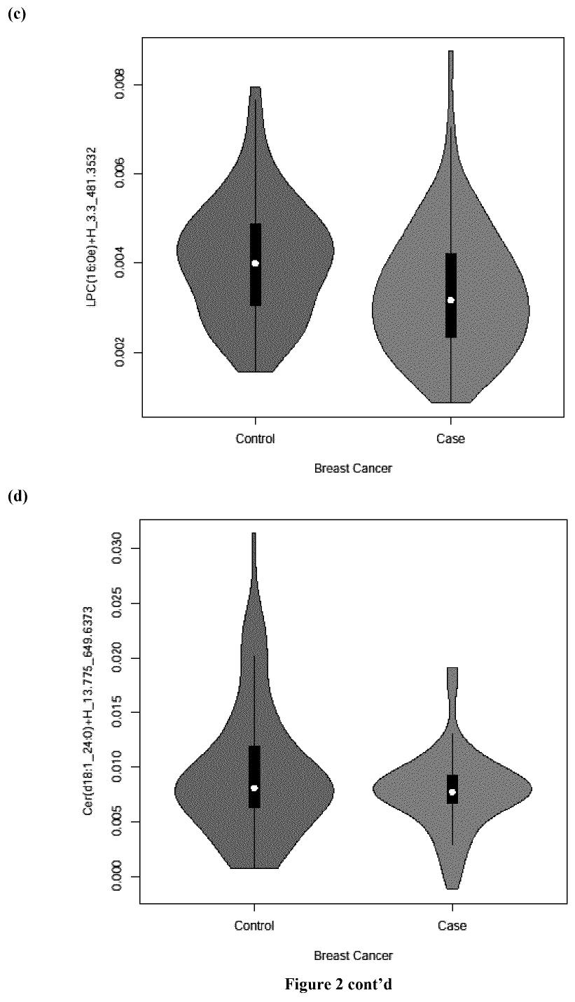

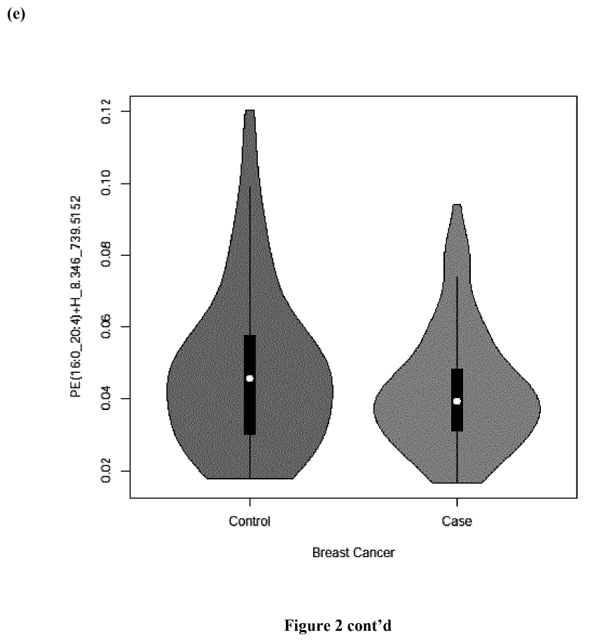

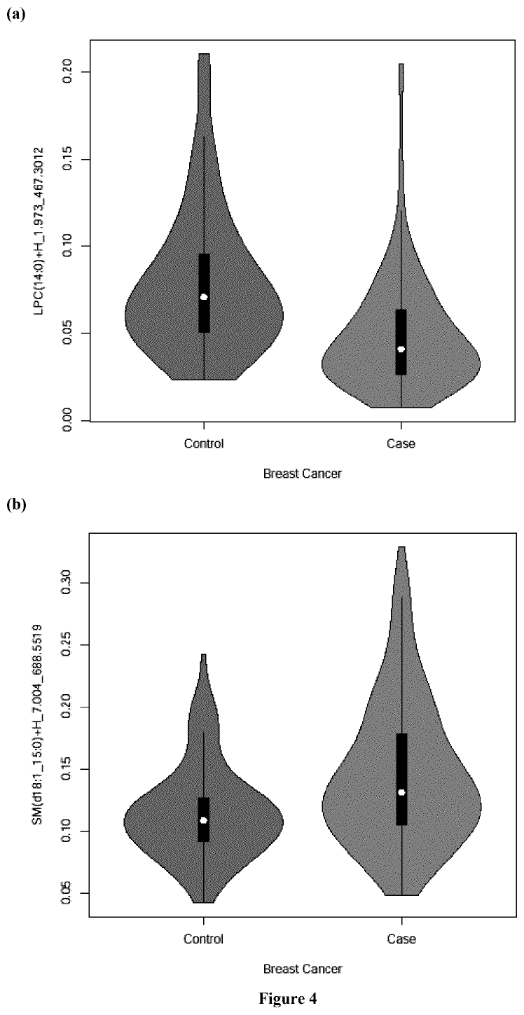

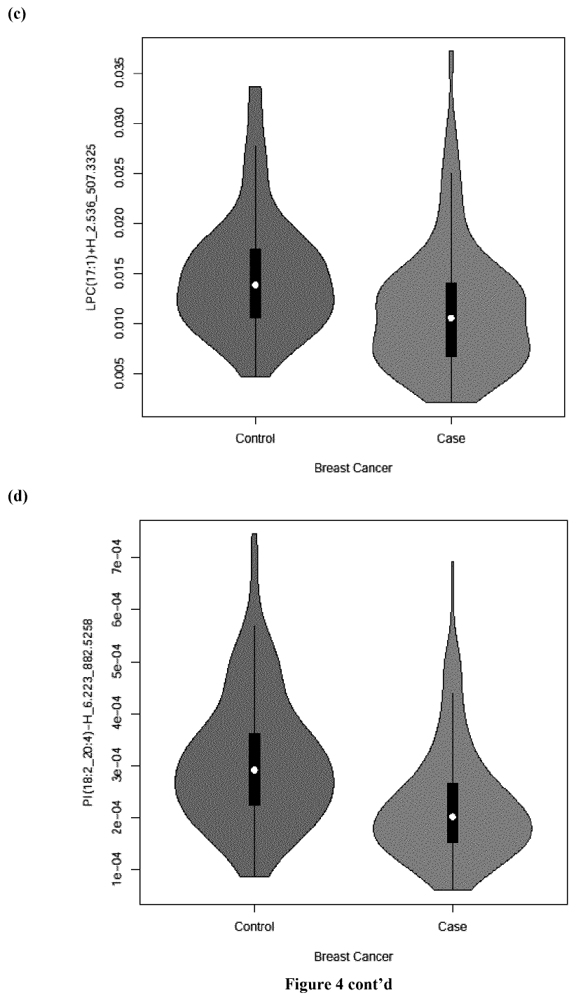

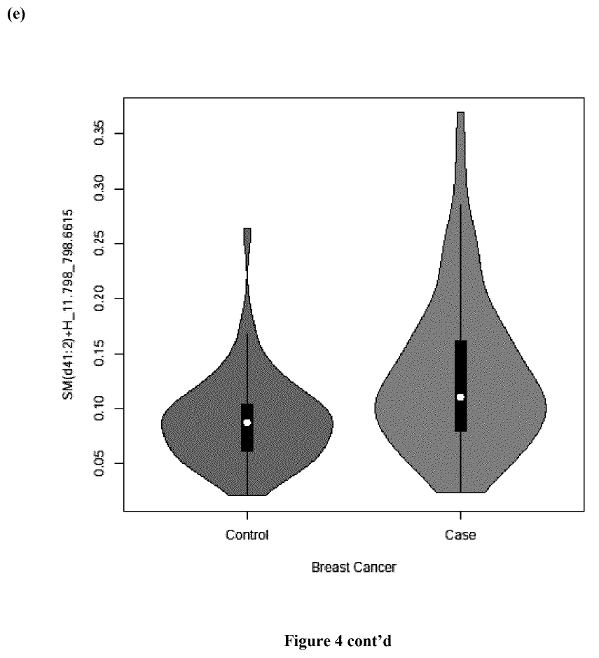

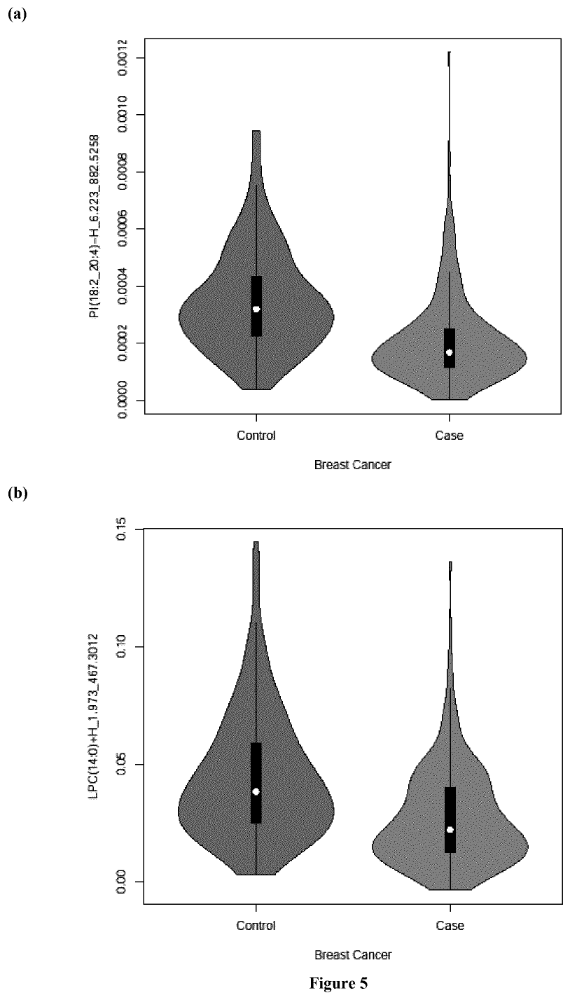

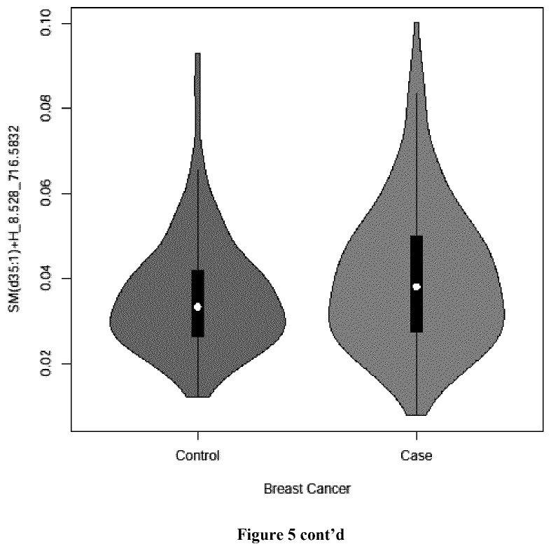

The outcome for each lipid, was an estimate of its relative influence or feature importance. In order to protect against over-fitting, variable selection was achieved by selecting the five lipids with greatest estimated relative influence. One or more of these were excluded if their estimated relative influence was <5%. Variables (lipids) selected and their estimated relative influence/feature importance were tabulated (Tables 8-12). Violin plots show the distribution of the lipid concentrations, by case/control status (FIGS. 1-5).

### Machine Learning Results for Data Set 1

TABLE 8
Five lipids with highest estimated relative
influence/feature importance

| Feature | Feature Importance |
|---|---|
| LPC(14:0) + H_1.973_467.3012 | 0.200695 |
| TG(16:0_18:1_23:0) + NH4_17.622_930.8615 | 0.118482 |
| LPC(18:3) + H_1.907_517.3168 | 0.083962 |
| PS(18:0_20:4) − H_8.806_811.5363 | 0.050535 |
| PC(18:1e_20:4) + H_10.456_793.5985 | 0.045696 |

### Machine Learning Results for Data Set 2

TABLE 9
Five lipids with highest estimated relative
influence/feature importance

| Feature | Feature Importance |
|---|---|
| PI(18:2_20:4) − H_6.223_882.5258 | 0.234965 |
| LPE(22:6) − H_2.243_525.2855 | 0.087382 |
| LPC(16:0e) + H_3.3_481.3532 | 0.065528 |
| Cer(d18:1_24:0) + H_13.775_649.6373 | 0.055938 |
| PE(16:0_20:4) + H_8.346_739.5152 | 0.051667 |

### Machine Learning Results for Data Set 3

TABLE 10
Five lipids with highest estimated relative
influence/feature importance

| Feature | Feature Importance |
|---|---|
| PI(18:2_20:4) − H_6.223_882.5258 | 0.206972816 |
| PE(16:0p_18:2) + H_9.427_699.5203 | 0.111700063 |
| PI(18:0_18:1) − H_10.026_864.5728 | 0.078094035 |
| PS(18:0_22:6) − H_8.422_835.5363 | 0.073597258 |
| PE(20:1p_18:2) + H_11.17_753.5672 | 0.057497532 |

### Machine Learning Results for Data Set 4

TABLE 11
Five lipids with highest estimated relative
influence/feature importance

| Feature | Feature Importance |
|---|---|
| LPC(14:0) + H_1.973_467.3012 | 0.094207 |
| SM(d18:1_15:0) + H_7.004_688.5519 | 0.087243 |
| LPC(17:1) + H_2.536_507.3325 | 0.076116 |
| PI(18:2_20:4) − H_6.223_882.5258 | 0.075970 |
| SM(d41:2) + H_11.798_798.6615 | 0.055660 |

### Machine Learning Results for Data Set 234 (Training Set)

TABLE 12
Five lipids with highest estimated relative
influence/feature importance

| Feature | Feature Importance |
|---|---|
| PI(18:2_20:4) − H_6.223_882.5258 | 0.177488 |
| LPC(14:0) + H_1.973_467.3012 | 0.072399 |
| SM(d35:1) + H_8.528_716.5832 | 0.072387 |
| LPC(18:1e) + H_3.411_507.3689 | 0.034876 |
| PS(18:0_20:4) − H_8.806_811.5363 | 0.034358 |

### Candidate Signatures

For each lipid-set, a logistic model was fitted, by regressing case/control status on lipids selected. Some models were simplified if there was no loss in goodness of fit, for example when two highly correlated lipids were included. The resultant models are given in Tables 13-17.

TABLE 13
Candidate signature arising from data set 1

| | | Std. | | |
|---|---|---|---|---|
| | Estimate | Error | z value | Pr(>\|z\|) |
| (Intercept) | 2.20 | 0.91 | 2.42 | 0.015619 |
| LPC(14:0) + H_1.973_467.3012 | −369.35 | 147.62 | −2.50 | 0.012351 |
| PS(18:0_20:4) − H_8.806_811.5363 | 61.31 | 21.38 | 2.87 | 0.004136 |
| TG(16:0_18:1_23:0) + NH4_17.622_930.8615 | −760.03 | 249.47 | −3.05 | 0.002315 |

TABLE 14
Candidate signature arising from data set 2

| | | Std. | | |
|---|---|---|---|---|
| | Estimate | Error | z value | Pr(>\|z\|) |
| (Intercept) | 1.05 | 0.59 | 1.78 | 0.075118 |
| LPC(16:0e) + H_3.3_481.3532 | −744.89 | 170.74 | −4.36 | 1.28E−05 |
| LPE(22:6) − H_2.243_525.2855 | 331.13 | 70.52 | 4.70 | 2.66E−06 |
| PI(18:2_20:4)-H_6.223_882.5258 | −3696.53 | 814.09 | −4.54 | 5.61E−06 |

TABLE 15
Candidate signature arising from data set 3

| | Estimate | Std. Error | z value | Pr(>\|z\|) |
|---|---|---|---|---|
| (Intercept) | 7.11 | 1.07 | 6.66 | 2.69E−11 |
| PE(16:0p_18:2) + H_9.427_699.5203 | −77.85 | 28.80 | −2.70 | 0.006876 |
| PE(20:1p_18:2) + H_11.17_753.5672 | −165.86 | 66.80 | −2.48 | 0.013034 |
| PI(18:0_18:1) − H_10.026_864.5728 | −111.39 | 47.94 | −2.32 | 0.020153 |
| PI(18:2_20:4) − H_6.223_882.5258 | −10369.53 | 2411.57 | −4.30 | 1.71E−05 |
| PS(18:0_22:6) − H_8.422_835.5363 | −186.23 | 62.53 | −2.98 | 0.002898 |

TABLE 16
Candidate signature arising from data set 4

| | | Std. | | |
|---|---|---|---|---|
| | Estimate | Error | z value | Pr(>\|z]) |
| (Intercept) | 0.47 | 0.53 | 0.89 | 0.375192 |
| LPC(14:0) + H_1.973_467.3012 | −34.17 | 5.61 | −6.10 | 1.09E−09 |
| PI(18:2_20:4) − H_6.223_882.5258 | −4651.38 | 1335.37 | −3.48 | 0.000495 |
| SM(d18:1_15:0) + H_7.004_688.5519 | 27.99 | 4.62 | 6.05 | 1.42E−09 |

TABLE 17
Candidate signature arising from data set 234 (Training)

| | | Std. | | |
|---|---|---|---|---|
| | Estimate | Error | z value | Pr(>\|z\|) |
| (Intercept) | 1.44 | 0.36 | 3.96 | 7.62E−05 |
| LPC(14:0) + H_1.973_467.3012 | −32.34 | 5.34 | −6.05 | 1.41E−09 |
| PI(18:2_20:4) − H_6.223_882.5258 | −4536.83 | 713.03 | −6.36 | 1.98E−10 |
| SM(d35:1) + H_8.528_716.5832 | 32.17 | 8.11 | 3.97 | 7.31E−05 |

### Test Performance Calculations

The test performance of each candidate signature arising from a single dataset was determined by applying it to the remaining three individual datasets, as independent patient sets.

The test performance of the candidate signature derived from Dataset 234 (Training Set) was determined by application to Dataset 1 and Dataset 234 (Validation Set). Test performance calculations took the following form. The sensitivity, specificity, positive predictive value (PPV) and negative predictive value (NPV) were calculated for the range of predicted probability thresholds. A table was generated to summarise performance over a range of classification thresholds, to allow greater or lesser weight to be applied to true positive versus false positive findings. A Receiver Operating Characteristic (ROC) curve was generated to plot sensitivity as a function of (1-specificity). In addition, the Area Under the Curve (AUC) was estimated.

### Performance of Candidate Signature 1

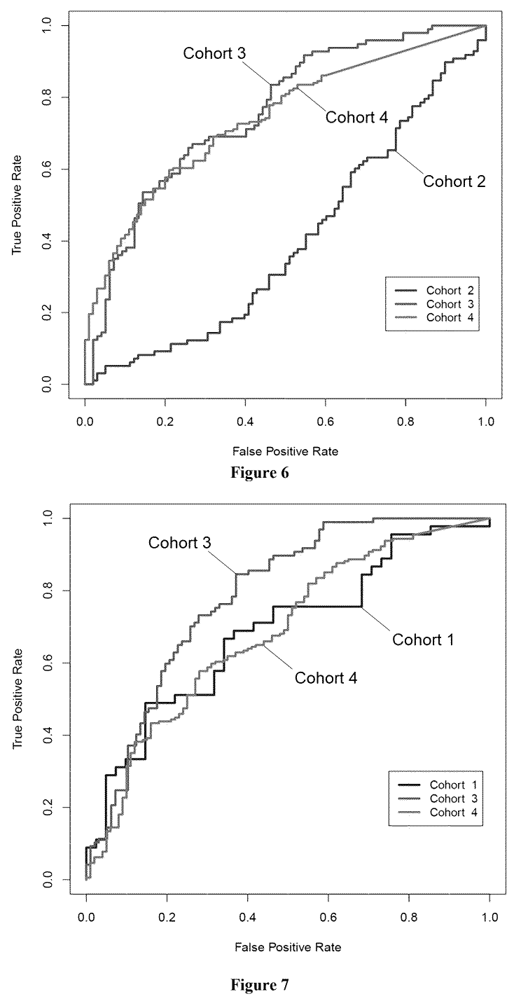

This signature was supported by data in cohorts 3 and 4 (AUC=0.76 and 0.74 respectively). FIG. 6 shows the overlaid ROC curve.

### Performance of Candidate Signature 2

This signature was supported by data in cohort 3 (AUC=0.79). Performance in cohorts 1 and 4 was weaker (AUC-0.69 and 0.68 respectively), but still supported. FIG. 7 shows the overlaid ROC curve.

### Performance of Candidate Signature 3

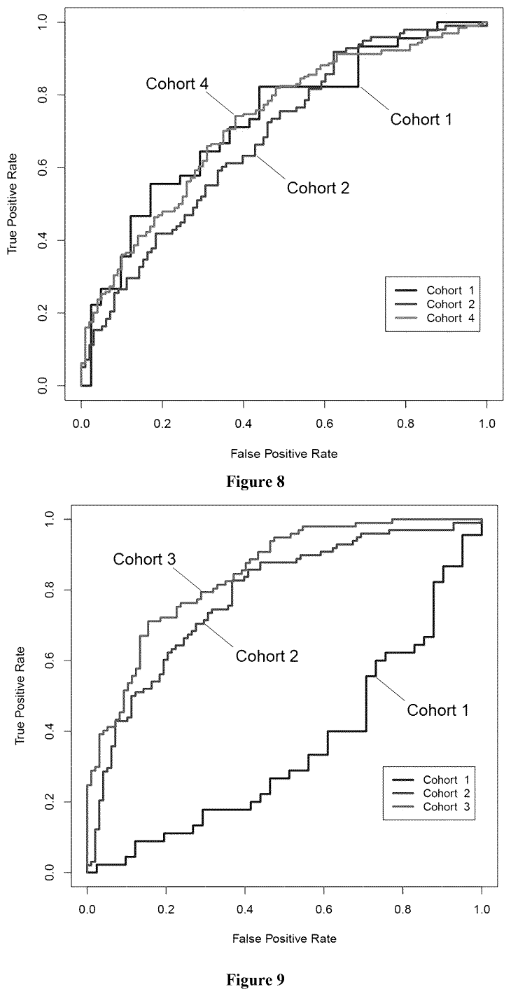

This signature was supported by data in cohorts 1 and 4 (AUC=0.73 and 0.72 respectively). Performance in cohort 2 was slightly weaker (AUC-0.68), but still supported. FIG. 8 shows the overlaid ROC curve.

### Performance of Candidate Signature 4

This signature was supported by data in cohorts 2 and 3 (AUC=0.78 and 0.85 respectively). FIG. 9 shows the overlaid ROC curve.

### Performance of Candidate Signature 234

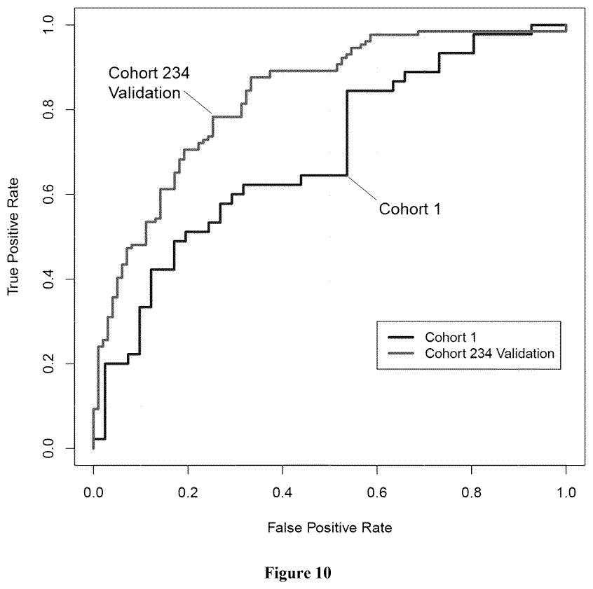

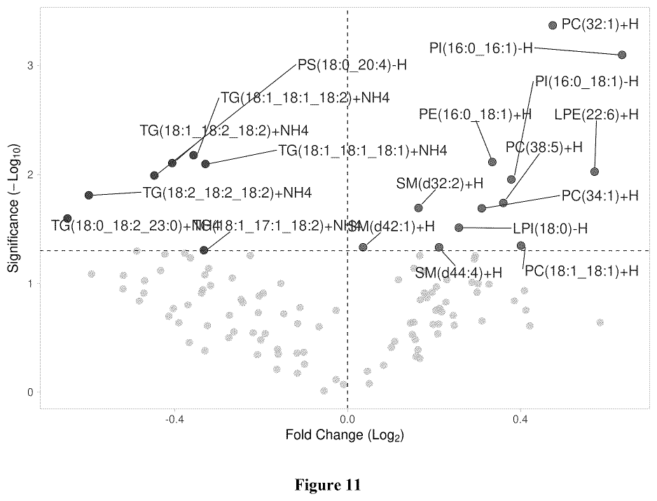

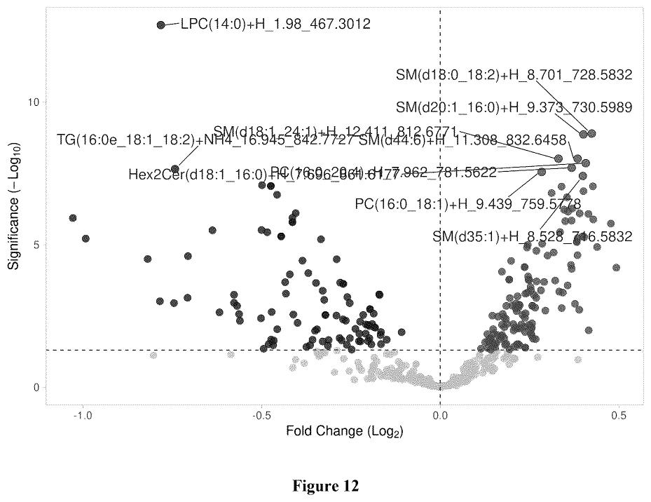

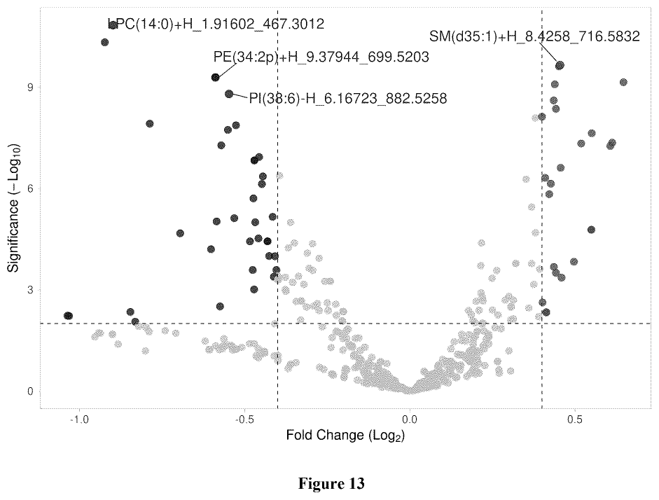

This signature was very close in composition to candidate signature 4. It was supported by data in dataset 234 Validation (AUC-0.83). Performance in cohort 1 was weaker (AUC=0.69), but still supported. FIG. 10 shows the overlaid ROC curve.

### Example 2

In the present Example, the inventors processed and analysed 4 cohorts containing blood plasma, sequentially over time, with the first cohort available in 2018 and Cohort 4 available in 2021 (as per the above cohorts for Example 1). Over this time, various statistical analyses were undertaken, with the goal to find panels of lipids that differentiate cancer from control samples in the cohorts provided.

### Study Design

The study design, number and description of cases in each cohort, the processing batches (described as Set 1 through Set 6), as well as clinical characteristics are described in Example 1 above.

### General Consideration for Data Analysis

At all stages of the data analysis the emphasis was on the diagnosis of early stage cancer (Stages I and II): in earlier phases of the analysis stage specific differences were also considered.

### Datasets Used and Improvements Over Time in Data Structure

A merged, manually curated dataset of common lipids was compiled in 2020 after Cohorts 1-3 became available; this improved version of the data contains the ˜450 lipid identifiers which could be matched across all later cohorts, and to which Cohort 4 was added subsequently. This is referred to as the BCAL lipid database.

### Data Merging and Lipid Identifiers

Prior to the manually curated BCAL lipid database, data merging was done at the bioinformatics analysis level, by a combination of matching either exact lipid ions wherever possible, and in addition employing m/z values matching combined with a retention time ordering. That approach was employed for the original three cohorts, and was superseded by using the lipid database once available.

### Data Processing

For all analyses, the following steps were taken:
lipids with low average values <10⁻⁵ were removed
lipids with missing values were discarded (this only impacts the earlier analyses since the lipid database does not contain missing values)
the remaining data was normalised to adjust for possible differences in total sample amount
a natural log transform was applied
the distribution of the resulting data was checked visually with boxplots and density plots
the overall clustering was checked by generating principal component plots
no outliers were removed

### Batch Normalisation

Batch processing differences were apparent whenever different datasets were combined and thus batch normalisation was evaluated. Starting with Cohort 2, Set 3 batch sample repeats became available in addition to technical injection repeats, and they were used to assess the usefulness of batch normalisation using repeats correlations and coefficients of variation. An adapted version of internal reference scaling (IRS) normalisation was found to give good performance (Plubell, 2017) and was used subsequently for batch normalisation.

### Variable Selection

At each stage of the analysis, lipid biomarkers were selected based on their ability to distinguish between control and cancer in the datasets currently available, with the selections validated in subsequent sets.

The selection of the initial 18 lipid biomarkers was carried out using Set 1 and Set 2, merged bioinformatically as described above (see Table 18 below). Mixed effects models with random batch effects were run to find differentially expressed markers for the available sets using the nlme R package implementation (Pinheiro, 2000).

TABLE 18
Initial 18 Step 1 - Set 1 and Set 2 Panel

| Skyline ID (cohort3; sum comp; | Skyline ID (cohort4; LI + AD_RT | |
|---|---|---|
| LI + AD_RT 3dp_MzNoAD 4dp) | 3dp_MzNoAD 4dp) | m/z |
| LPE(22:6) − H_2.243_525.2855 | LPE(22:6) − H_2.213_525.2855 | 525.2855 |
| PC(32:1) + H_7.891_731.5465 | PC(16:0_16:1) + H_7.814_731.5465 | 731.5465 |
| PC(34:1) + H_9.435_759.5778 | PC(16:0_18:1) + H_9.363_759.5778 | 759.5778 |
| PC(36:2) + H_9.593_785.5935 | PC(18:1_18:1) + H_9.504_785.5935 | 785.5935 |
| PC(36:2) + H_9.901_785.5935 | PC(18:0_18:2) + H_9.826_785.5935 | 785.5935 |
| PC(36:2) + H_9.901_785.5935 | PC(18:1_18:1) + H_9.826_785.5935 | 785.5935 |
| PC(38:5) + H_7.596_807.5778 | PC(20:3_18:2) + H_7.52_807.5778 | 807.5778 |
| PC(38:5) + H_8.05_807.5778 | PC(18:1_20:4) + H_7.974_807.5778 | 807.5778 |
| PC(38:5) + H_8.076_807.5778_all | PC(18:1_20:4) + H_8.046_807.5778_all | 807.5778 |
| PC(38:5) + H_8.536_807.5778 | PC(18:0_20:5) + H_8.496_807.5778 | 807.5778 |
| PE(34:1) + H_9.855_717.5309 | PE(16:0_18:1) + H_9.807_717.5309 | 717.5309 |
| PE(34:1) − H_9.853_717.5309 | PE(16:0_18:1) − H_9.81_717.5309 | 717.5309 |
| PI(32:1) − H_6.993_808.5102 | PI(16:0_16:1) − H_6.924_808.5102 | 808.5102 |
| PI(34:1) − H_8.373_836.5415 | PI(16:0_18:1) − H_8.277_836.5415 | 836.5415 |
| PS(38:4) − H_10.319_811.5363 | PS(18:0_20:4) − H_10.279_811.5363 | 811.5363 |
| PS(38:4) − H_8.806_811.5363 | PS(18:0_20:4) − H_8.663_811.5363 | 811.5363 |
| SM(d32:2) + H_5.375_672.5206 | SM(d32:2) + H_5.312_672.5206 | 672.5206 |
| SM(d42:1) + H_13.686_814.6928 | SM(d18:1_24:0) + H_13.613_814.6928 | 814.6928 |
| SM(d42:1) + H_13.686_814.6928 | SM(d42:1) + H_13.613_814.6928 | 814.6928 |
| SM(d44:4) + H_11.511_836.6771 | SM(d44:4) + H_11.432_836.6771 | 836.6771 |
| SM(d44:4) + H_13.687_836.6771 | SM(d20:0_24:4) + H_13.603_836.6771 | 836.6771 |
| TG(53:4) + NH4_16.297_868.752 | TG(17:0_18:2_18:2) + NH4_16.308_868.752 | 868.752 |
| TG(54:3) + NH4_16.761_884.7833 | TG(18:1_18:1_18:1) + NH4_16.782_884.7833 | 884.7833 |
| TG(54:3) + NH4_16.761_884.7833 | TG(18:1_18:1_18:1) + NH4_16.782_884.7833 | 884.7833 |
| TG(54:4) + NH4_16.462_882.7676 | TG(18:1_18:1_18:2) + NH4_16.467_882.7676 | 882.7676 |
| TG(54:5) + NH4_16.152_880.752 | TG(18:1_18:2_18:2) + NH4_16.149_880.752 | 880.752 |
| TG(54:5) + NH4_16.154_880.752 | TG(18:2_18:2_18:1) + NH4_16.157_880.752 | 880.752 |
| TG(54:6) + H_16.503_878.7363 | TG(18:1_18:2_18:3) + H_16.493_878.7363 | 878.7363 |
| TG(54:6) + NH4_15.783_878.7363 | TG(18:1_18:2_18:3) + NH4_15.78_878.7363 | 878.7363 |
| TG(54:6) + NH4_16.029_878.7363 | TG(18:1_18:2_18:3) + NH4_16.025_878.7363 | 878.7363 |
| TG(59:2) + NH4_17.624_956.8772 | TG(18:1_18:1_23:0) + NH4_17.647_956.8772 | 956.8772 |

An initial course filter was run to retain all markers that showed evidence of differential expression between cither Control-Early or Control-Stage1-Stage2, using both ANOVA or mixed effects models with batch effect.

Any lipids with a Benjamini and Hochberg adjusted p-value <0.05 (Benjamini, 1995) for any of these models were selected, resulting in a subset of 110 markers of initial interest.

This filtered set of markers generated to run differential expression between Control and Early cancer samples, selecting the top differentially expressed markers based on p-value<0.05 and fold change criteria only (Initial panel of 18—“Initial 18 Step 1 Set 1 and Set 2”). The volcano plots below show the lipid fold change and p-values (Goedhart, 2020), with the lipid ion identifiers as available at the time.

The steps above were repeated using the whole of Cohort 2 when Set 3 became available. After this process the initial panel of 18 was enhanced with 9 more markers using Set 2 and Set 3 data, resulting in the panel “Initial 18+9 more Cohort2 NewVS”, which represented an intermediate panel of interest (Table 19).

TABLE 19
Initial 18 + 9 more Cohort2 NewVS

| Skyline ID (cohort3; sum comp; | Skyline ID (cohort4; LI + AD_RT | |
|---|---|---|
| LI + AD_RT 3dp_MzNoAD 4dp) | 3dp_MzNoAD 4dp) | m/z |
| Cer(d42:0) + H_14.782_651.6529 | Cer(d18:0_24:0) + H_14.782_651.6529 | 651.6529 |
| LPE(22:6) − H_2.243_525.2855 | LPE(22:6) − H_2.213_525.2855 | 525.2855 |
| LPI(20:4) − H_2.036_620.2962 | LPI(20:4) − H_2.006_620.2962 | 620.2962 |
| PC(32:1) + H_7.891_731.5465 | PC(16:0_16:1) + H_7.814_731.5465 | 731.5465 |
| PC(34:1) + H_9.435_759.5778 | PC(16:0_18:1) + H_9.363_759.5778 | 759.5778 |
| PC(35:4) + H_7.246_767.5465 | PC(15:0_20:4) + H_7.184_767.5465 | 767.5465 |
| PC(36:2) + H_9.593_785.5935 | PC(18:1_18:1) + H_9.504_785.5935 | 785.5935 |
| PC(36:2) + H_9.901_785.5935 | PC(18:0_18:2) + H_9.826_785.5935 | 785.5935 |
| PC(36:2) + H_9.901_785.5935 | PC(18:1_18:1) + H_9.826_785.5935 | 785.5935 |
| PC(38:5) + H_7.596_807.5778 | PC(20:3_18:2) + H_7.52_807.5778 | 807.5778 |
| PC(38:5) + H_8.05_807.5778 | PC(18:1_20:4) + H_7.974_807.5778 | 807.5778 |
| PC(38:5) + H_8.076_807.5778_all | PC(18:1_20:4) + H_8.046_807.5778_all | 807.5778 |
| PC(38:5) + H_8.536_807.5778 | PC(18:0_20:5) + H_8.496_807.5778 | 807.5778 |
| PE(34:1) + H_9.855_717.5309 | PE(16:0_18:1) + H_9.807_717.5309 | 717.5309 |
| PE(34:1) − H_9.853_717.5309 | PE(16:0_18:1) − H_9.81_717.5309 | 717.5309 |
| PE(38:4) + H_10.056_767.5465 | PE(18:0_20:4) + H_10.003_767.5465 | 767.5465 |
| PE(38:4) − H_10.055_767.5465 | PE(18:0_20:4) − H_10.016_767.5465 | 767.5465 |
| PE(38:4p) + H_10.304_751.5516 | PE(16:0p_22:4) + H_10.247_751.5516 | 751.5516 |
| PE(38:5e) + H_10.888_751.5516 | PE(18:1e_20:4) + H_10.841_751.5516 | 751.5516 |
| PE(38:5e) − H_10.888_751.5516 | PE(18:1e_20:4) − H_10.844_751.5516 | 751.5516 |
| PE(40:5p) + H_10.434_777.5672 | PE(18:1p_22:4) + H_10.384_777.5672 | 777.5672 |
| PE(40:6e) − H_10.941_777.5672 | PE(18:1e_22:5) − H_10.867_777.5672 | 777.5672 |
| PE(40:6e) − H_10.941_777.5672 | PE(18:1e_22:5) − H_10.867_777.5672 | 777.5672 |
| PI(32:1) − H_6.993_808.5102 | PI(16:0_16:1) − H_6.924_808.5102 | 808.5102 |
| PI(34:1) − H_8.373_836.5415 | PI(16:0_18:1) − H_8.277_836.5415 | 836.5415 |
| PI(38:6) − H_6.223_882.5258 | PI(18:2_20:4) − H_6.167_882.5258 | 882.5258 |
| PI(38:6) − H_6.759_882.5258 | PI(16:0_22:6) − H_6.708_882.5258 | 882.5258 |
| PS(38:4) − H_10.319_811.5363 | PS(18:0_20:4) − H_10.279_811.5363 | 811.5363 |
| PS(38:4) − H_8.806_811.5363 | PS(18:0_20:4) − H_8.663_811.5363 | 811.5363 |
| PS(40:7) − H_8.792_833.5207 | PS(20:3_20:4) − H_8.628_833.5207 | 833.5207 |
| SM(d32:2) + H_5.375_672.5206 | SM(d32:2) + H_5.312_672.5206 | 672.5206 |
| SM(d42:1) + H_13.686_814.6928 | SM(d18:1_24:0) + H_13.613_814.6928 | 814.6928 |
| SM(d42:1) + H_13.686_814.6928 | SM(d42:1) + H_13.613_814.6928 | 814.6928 |
| SM(d44:4) + H_11.511_836.6771 | SM(d44:4) + H_11.432_836.6771 | 836.6771 |
| SM(d44:4) + H_13.687_836.6771 | SM(d20:0_24:4) + H_13.603_836.6771 | 836.6771 |
| TG(53:4) + NH4_16.297_868.752 | TG(17:0_18:2_18:2) + NH4_16.308_868.752 | 868.752 |
| TG(54:3) + NH4_16.761_884.7833 | TG(18:1_18:1_18:1) + NH4_16.782_884.7833 | 884.7833 |
| TG(54:3) + NH4_16.761_884.7833 | TG(18:1_18:1_18:1) + NH4_16.782_884.7833 | 884.7833 |
| TG(54:4) + NH4_16.462_882.7676 | TG(18:1_18:1_18:2) + NH4_16.467_882.7676 | 882.7676 |
| TG(54:5) + NH4_16.152_880.752 | TG(18:1_18:2_18:2) + NH4_16.149_880.752 | 880.752 |
| TG(54:5) + NH4_16.154_880.752 | TG(18:2_18:2_18:1) + NH4_16.157_880.752 | 880.752 |
| TG(54:6) + H_16.503_878.7363 | TG(18:1_18:2_18:3) + H_16.493_878.7363 | 878.7363 |
| TG(54:6) + NH4_15.783_878.7363 | TG(18:1_18:2_18:3) + NH4_15.78_878.7363 | 878.7363 |
| TG(54:6) + NH4_16.029_878.7363 | TG(18:1_18:2_18:3) + NH4_16.025_878.7363 | 878.7363 |
| TG(59:2) + NH4_17.624_956.8772 | TG(18:1_18:1_23:0) + NH4_17.647_956.8772 | 956.8772 |

After the completion of Cohort 3 (Set 4 and Set 5), differential expression using ANOVA on merged and IRS batch normalised data was used, and the top up and down regulated lipids were added to the previously existing panel (400 Reoptimised lipid panel of 18-“Cohort 2-3 final”; See Table 21 below). Similarly, the distribution of fold changes and p-values is visualised on the volcano plot below. The intermediate additions of 18 most differentially expressed markers for Cohort 3 (top 9 most up-regulated and top 9 most down regulated based on fold change cancer/control from amongst the differentially expressed lipids based on p-value <0.05) were retained as panel “Cohort 3 additions Cohort 3 Plsda add 9 up 9 down” (Table 20).

TABLE 20
Cohort 3 additions Cohort 3 Plsda add 9 up 9 down

| Skyline ID (cohort3; sum comp; | Skyline ID (cohort4; LI + AD_RT | |
|---|---|---|
| LI + AD_RT 3dp_MzNoAD 4dp) | 3dp_MzNoAD 4dp) | m/z |
| DG(34:2) + NH4_11.381_592.5067 | DG(16:0_18:2) + NH4_11.35_592.5067 | 592.5067 |
| Hex2Cer(d34:1) + H_7.694_861.6177 | Hex2Cer(d18:1_16:0) + H_7.677_861.6177 | 861.6177 |
| Hex2Cer(d42:2) − H_12.27_971.7273 | Hex2Cer(d18:1_24:1) − H_12.244_971.7273 | 971.7273 |
| LPC(14:0) + H_1.973_467.3012 | LPC(14:0) + H_1.916_467.3012 | 467.3012 |
| PC(36:3) + H_7.966_783.5778 | PC(18:1_18:2) + H_7.899_783.5778 | 783.5778 |
| PC(36:3) + H_8.49_783.5778 | PC(18:1_18:2) + H_8.502_783.5778 | 783.5778 |
| PC(37:4) + H_8.471_795.5778 | PC(17:0_20:4) + H_8.377_795.5778 | 795.5778 |
| PC(37:4) + H_8.786_795.5778 | PC(17:0_20:4) + H_8.713_795.5778 | 795.5778 |
| PC(38:5) + H_7.596_807.5778 | PC(20:3_18:2) + H_7.52_807.5778 | 807.5778 |
| PC(38:5) + H_8.05_807.5778 | PC(18:1_20:4) + H_7.974_807.5778 | 807.5778 |
| PC(38:5) + H_8.076_807.5778_all | PC(18:1_20:4) + H_8.046_807.5778_all | 807.5778 |
| PC(38:5) + H_8.536_807.5778 | PC(18:0_20:5) + H_8.496_807.5778 | 807.5778 |
| SM(d36:2) + H_7.99_728.5832 | SM(d18:0_18:2) + H_7.931_728.5832 | 728.5832 |
| SM(d38:4) + H_9.342_752.5832 | SM(d38:4) + H_9.269_752.5832 | 752.5832 |
| SM(d41:3) + H_10.493_796.6458 | SM(d41:3) + H_10.41_796.6458 | 796.6458 |
| TG(52:3e) + NH4_16.938_842.7727 | TG(16:0e_18:1_18:2) + NH4_16.944_842.7727 | 842.7727 |
| TG(56:1) + NH4_17.513_916.8459 | TG(16:0_18:1_22:0) + NH4_17.517_916.8459 | 916.8459 |
| TG(58:1) + NH4_17.517_944.8772 | TG(18:0_18:1_22:0) + NH4_17.521_944.8772 | 944.8772 |
| TG(58:2) + NH4_17.521_942.8615 | TG(18:1_18:1_22:0) + NH4_17.523_942.8615 | 942.8615 |
| TG(58:3) + NH4_17.293_940.8459 | TG(18:1_18:1_22:1) + NH4_17.3_940.8459 | 940.8459 |
| TG(60:1) + NH4_17.971_972.9085 | TG(18:0_18:1_24:0) + NH4_17.983_972.9085 | 972.9085 |
| TG(62:2) + NH4_17.956_998.9241 | TG(26:0_18:1_18:1) + NH4_17.972_998.9241 | 998.9241 |

TABLE 21
BCAL current panel of 18 400 Reoptimised (Cohort 2-3 final)

| Skyline ID (cohort3; sum comp; | Skyline ID (cohort4; LI + AD_RT | |
|---|---|---|
| LI + AD_RT 3dp_MzNoAD 4dp) | 3dp_MzNoAD 4dp) | m/z |
| LPC(14:0) + H_1.973_467.3012 | LPC(14:0) + H_1.916_467.3012 | 467.3012 |
| PC(32:1) + H_7.891_731.5465 | PC(16:0_16:1) + H_7.814_731.5465 | 731.5465 |
| PC(36:2) + H_9.593_785.5935 | PC(18:1_18:1) + H_9.504_785.5935 | 785.5935 |
| PC(36:2) + H_9.901_785.5935 | PC(18:0_18:2) + H_9.826_785.5935 | 785.5935 |
| PC(36:2) + H_9.901_785.5935 | PC(18:1_18:1) + H_9.826_785.5935 | 785.5935 |
| PC(38:5) + H_7.596_807.5778 | PC(20:3_18:2) + H_7.52_807.5778 | 807.5778 |
| PC(38:5) + H_8.05_807.5778 | PC(18:1_20:4) + H_7.974_807.5778 | 807.5778 |
| PC(38:5) + H_8.076_807.5778_all | PC(18:1_20:4) + H_8.046_807.5778_all | 807.5778 |
| PC(38:5) + H_8.536_807.5778 | PC(18:0_20:5) + H_8.496_807.5778 | 807.5778 |
| PE(34:1) + H_9.855_717.5309 | PE(16:0_18:1) + H_9.807_717.5309 | 717.5309 |
| PE(34:1) − H_9.853_717.5309 | PE(16:0_18:1) − H_9.81_717.5309 | 717.5309 |
| PI(34:1) − H_8.373_836.5415 | PI(16:0_18:1) − H_8.277_836.5415 | 836.5415 |
| PS(38:4) − H_10.319_811.5363 | PS(18:0_20:4) − H_10.279_811.5363 | 811.5363 |
| PS(38:4) − H_8.806_811.5363 | PS(18:0_20:4) − H_8.663_811.5363 | 811.5363 |
| SM(d36:2) + H_7.99_728.5832 | SM(d18:0_18:2) + H_7.931_728.5832 | 728.5832 |
| SM(d38:4) + H_9.342_752.5832 | SM(d38:4) + H_9.269_752.5832 | 752.5832 |
| SM(d44:4) + H_11.511_836.6771 | SM(d44:4) + H_11.432_836.6771 | 836.6771 |
| SM(d44:4) + H_13.687_836.6771 | SM(d20:0_24:4) + H_13.603_836.6771 | 836.6771 |
| TG(52:3e) + NH4_16.938_842.7727 | TG(16:0e_18:1_18:2) + NH4_16.944_842.7727 | 842.7727 |
| TG(53:4) + NH4_16.297_868.752 | TG(17:0_18:2_18:2) + NH4_16.308_868.752 | 868.752 |
| TG(54:4) + NH4_16.462_882.7676 | TG(18:1_18:1_18:2) + NH4_16.467_882.7676 | 882.7676 |
| TG(54:5) + NH4_16.152_880.752 | TG(18:1_18:2_18:2) + NH4_16.149_880.752 | 880.752 |
| TG(54:5) + NH4_16.154_880.752 | TG(18:2_18:2_18:1) + NH4_16.157_880.752 | 880.752 |
| TG(54:6) + H_16.503_878.7363 | TG(18:1_18:2_18:3) + H_16.493_878.7363 | 878.7363 |
| TG(54:6) + NH4_15.783_878.7363 | TG(18:1_18:2_18:3) + NH4_15.78_878.7363 | 878.7363 |
| TG(54:6) + NH4_16.029_878.7363 | TG(18:1_18:2_18:3) + NH4_16.025_878.7363 | 878.7363 |
| TG(56:1) + NH4_17.513_916.8459 | TG(16:0_18:1_22:0) + NH4_17.517_916.8459 | 916.8459 |
| TG(58:2) + NH4_17.521_942.8615 | TG(18:1_18:1_22:0) + NH4_17.523_942.8615 | 942.8615 |
| TG(58:3) + NH4_17.293_940.8459 | TG(18:1_18:1_22:1) + NH4_17.3_940.8459 | 940.8459 |

Subsequent filtering to smaller panels was done by a combination of stepwise regression or further dropping lipids without significant contributions to logistic models differentiating between control and early stages, using the implementations available in the core stats R package (Dobson, 1990). This resulted in the selection of the smaller subsets of lipids (9 Lipid Panel of the initial 18-“18-9 lipids Restrict9Lipids”, Subset of 10 from the 400 optimised panel of 18, “Top 10 optimised for patent Top10 Coeff5050train”; see Tables 22 and 23 below, and the subset “Cohort 2 optimised” obtained in the same manner from the Cohort 2 lipid selection “Initial 18+9 more Cohort2 NewVS”; see Table 24 below).

TABLE 22
9 Lipid Panel of the initial 18 - “18-9 lipids Restrict9Lipids”

| Skyline ID (cohort3; sum comp; | Skyline ID (cohort4; LI + AD_RT | |
|---|---|---|
| LI + AD_RT 3dp_MzNoAD 4dp) | 3dp_MzNoAD 4dp) | m/z |
| PC(32:1) + H_7.891_731.5465 | PC(16:0_16:1) + H_7.814_731.5465 | 731.5465 |
| PC(34:1) + H_9.435_759.5778 | PC(16:0_18:1) + H_9.363_759.5778 | 759.5778 |
| PC(36:2) + H_9.593_785.5935 | PC(18:1_18:1) + H_9.504_785.5935 | 785.5935 |
| PC(36:2) + H_9.901_785.5935 | PC(18:0_18:2) + H_9.826_785.5935 | 785.5935 |
| PC(36:2) + H_9.901_785.5935 | PC(18:1_18:1) + H_9.826_785.5935 | 785.5935 |
| PE(34:1) + H_9.855_717.5309 | PE(16:0_18:1) + H_9.807_717.5309 | 717.5309 |
| PE(34:1) − H_9.853_717.5309 | PE(16:0_18:1) − H_9.81_717.5309 | 717.5309 |
| PI(32:1) − H_6.993_808.5102 | PI(16:0_16:1) − H_6.924_808.5102 | 808.5102 |
| PS(38:4) − H_10.319_811.5363 | PS(18:0_20:4) − H_10.279_811.5363 | 811.5363 |
| PS(38:4) − H_8.806_811.5363 | PS(18:0_20:4) − H_8.663_811.5363 | 811.5363 |
| SM(d42:1) + H_13.686_814.6928 | SM(d18:1_24:0) + H_13.613_814.6928 | 814.6928 |
| SM(d42:1) + H_13.686_814.6928 | SM(d42:1) + H_13.613_814.6928 | 814.6928 |
| TG(54:5) + NH4_16.152_880.752 | TG(18:1_18:2_18:2) + NH4_16.149_880.752 | 880.752 |
| TG(54:5) + NH4_16.154_880.752 | TG(18:2_18:2_18:1) + NH4_16.157_880.752 | 880.752 |
| TG(59:2) + NH4_17.624_956.8772 | TG(18:1_18:1_23:0) + NH4_17.647_956.8772 | 956.8772 |

TABLE 23
Subset of 10 from the 400 optimised panel of 18, “Top
10 optimised for patent Top10Coeff5050train”

| Skyline ID (cohort3; sum comp; | Skyline ID (cohort4; LI + AD_RT | |
|---|---|---|
| LI + AD_RT 3dp_MzNoAD 4dp) | 3dp_MzNoAD 4dp) | m/z |
| LPC(14:0) + H_1.973_467.3012 | LPC(14:0) + H_1.916_467.3012 | 467.3012 |
| PC(32:1) + H_7.891_731.5465 | PC(16:0_16:1) + H_7.814_731.5465 | 731.5465 |
| PC(38:5) + H_7.596_807.5778 | PC(20:3_18:2) + H_7.52_807.5778 | 807.5778 |
| PC(38:5) + H_8.05_807.5778 | PC(18:1_20:4) + H_7.974_807.5778 | 807.5778 |
| PC(38:5) + H_8.076_807.5778_all | PC(18:1_20:4) + H_8.046_807.5778_all | 807.5778 |
| PC(38:5) + H_8.536_807.5778 | PC(18:0_20:5) + H_8.496_807.5778 | 807.5778 |
| PE(34:1) + H_9.855_717.5309 | PE(16:0_18:1) + H_9.807_717.5309 | 717.5309 |
| PE(34:1) − H_9.853_717.5309 | PE(16:0_18:1) − H_9.81_717.5309 | 717.5309 |
| PS(38:4) − H_10.319_811.5363 | PS(18:0_20:4) − H_10.279_811.5363 | 811.5363 |
| PS(38:4) − H_8.806_811.5363 | PS(18:0_20:4) − H_8.663_811.5363 | 811.5363 |
| SM(d36:2) + H_7.99_728.5832 | SM(d18:0_18:2) + H_7.931_728.5832 | 728.5832 |
| SM(d38:4) + H_9.342_752.5832 | SM(d38:4) + H_9.269_752.5832 | 752.5832 |
| TG(54:4) + NH4_16.462_882.7676 | TG(18:1_18:1_18:2) + NH4_16.467_882.7676 | 882.7676 |
| TG(56:1) + NH4_17.513_916.8459 | TG(16:0_18:1_22:0) + NH4_17.517_916.8459 | 916.8459 |
| TG(58:2) + NH4_17.521_942.8615 | TG(18:1_18:1_22:0) + NH4_17.523_942.8615 | 942.8615 |

TABLE 24
Cohort 2 optimised

| Skyline ID (cohort3; sum comp; | Skyline ID (cohort4; LI + AD_RT | |
|---|---|---|
| LI + AD_RT 3dp_MzNoAD 4dp) | 3dp_MzNoAD 4dp) | m/z |
| PC(35:4) + H_7.246_767.5465 | PC(15:0_20:4) + H_7.184_767.5465 | 767.5465 |
| PC(38:5) + H_7.596_807.5778 | PC(20:3_18:2) + H_7.52_807.5778 | 807.5778 |
| PC(38:5) + H_8.05_807.5778 | PC(18:1_20:4) + H_7.974_807.5778 | 807.5778 |
| PC(38:5) + H_8.076_807.5778_all | PC(18:1_20:4) + H_8.046_807.5778_all | 807.5778 |
| PC(38:5) + H_8.536_807.5778 | PC(18:0_20:5) + H_8.496_807.5778 | 807.5778 |
| PE(34:1) + H_9.855_717.5309 | PE(16:0_18:1) + H_9.807_717.5309 | 717.5309 |
| PE(34:1) − H_9.853_717.5309 | PE(16:0_18:1) − H_9.81_717.5309 | 717.5309 |
| PE(38:4) + H_10.056_767.5465 | PE(18:0_20:4) + H_10.003_767.5465 | 767.5465 |
| PE(38:4) − H_10.055_767.5465 | PE(18:0_20:4) − H_10.016_767.5465 | 767.5465 |
| PE(40:5p) + H_10.434_777.5672 | PE(18:1p_22:4) + H_10.384_777.5672 | 777.5672 |
| PE(40:6e) − H_10.941_777.5672 | PE(18:1e_22:5) − H_10.867_777.5672 | 777.5672 |
| PE(40:6e) − H_10.941_777.5672 | PE(18:1e_22:5) − H_10.867_777.5672 | 777.5672 |
| PI(32:1) − H_6.993_808.5102 | PI(16:0_16:1) − H_6.924_808.5102 | 808.5102 |
| PI(38:6) − H_6.223_882.5258 | PI(18:2_20:4) − H_6.167_882.5258 | 882.5258 |
| PI(38:6) − H_6.759_882.5258 | PI(16:0_22:6) − H_6.708_882.5258 | 882.5258 |
| PS(38:4) − H_10.319_811.5363 | PS(18:0_20:4) − H_10.279_811.5363 | 811.5363 |
| PS(38:4) − H_8.806_811.5363 | PS(18:0_20:4) − H_8.663_811.5363 | 811.5363 |
| PS(40:7) − H_8.792_833.5207 | PS(20:3_20:4) − H_8.628_833.5207 | 833.5207 |
| SM(d44:4) + H_11.511_836.6771 | SM(d44:4) + H_11.432_836.6771 | 836.6771 |
| SM(d44:4) + H_13.687_836.6771 | SM(d20:0_24:4) + H_13.603_836.6771 | 836.6771 |

Cohort 4 was not used directly for variable selection, but consistency of fold change Control/Cancer across all cohorts was used to select a smaller panel of 6 lipid biomarkers from amongst the 400Reoptimised panel showing consistent performance in all batches. (“Panel of 6 FourMarkers”: see Table 25).

TABLE 25
Panel of 6 FourMarkers

| Skyline ID (cohort3; sum comp; | Skyline ID (cohort4; LI + AD_RT | |
|---|---|---|
| LI + AD_RT 3dp_MzNoAD 4dp) | 3dp_MzNoAD 4dp) | m/z |
| LPC(14:0) + H_1.973_467.3012 | LPC(14:0) + H_1.916_467.3012 | 467.3012 |
| PE(34:1) + H_9.855_717.5309 | PE(16:0_18:1) + H_9.807_717.5309 | 717.5309 |
| PE(34:1) − H_9.853_717.5309 | PE(16:0_18:1) − H_9.81_717.5309 | 717.5309 |
| PS(38:4) − H_10.319_811.5363 | PS(18:0_20:4) − H_10.279_811.5363 | 811.5363 |
| PS(38:4) − H_8.806_811.5363 | PS(18:0_20:4) − H_8.663_811.5363 | 811.5363 |
| SM(d36:2) + H_7.99_728.5832 | SM(d18:0_18:2) + H_7.931_728.5832 | 728.5832 |
| TG(52:3e) + NH4_16.938_842.7727 | TG(16:0e_18:1_18:2) + NH4_16.944_842.7727 | 842.7727 |
| TG(58:3) + NH4_17.293_940.8459 | TG(18:1_18:1_22:1) + NH4_17.3_940.8459 | 940.8459 |

Additionally, the top 10 differentially abundant lipids for Cohort 4 were also identified by a combination of fold change and p-value cut-offs, similar to the previous cohorts, and they were used to select a panel of 6 lipids common with those 12 lipid biomarkers identified in Example 1 (“Common 6-ML_GBM”; see Table 26).

TABLE 26
Common 6 - ML GBM

| Lipid biomarker | m/z |
|---|---|
| LPC(14:0) + H_1.973_467.3012 | 467.3012 |
| LPE(22:6) − H_2.243_525.2855 | 525.2855 |
| PI(38:6) − H_6.759_882.5258 | 882.5258 |
| PE(34:2p) + H_9.427_699.5203 | 699.5203 |
| SM(d35:1) + H_8.528_716.5832 | 716.5832 |
| PS(38:4) − H_10.319_811.5363 | 811.5363 |
| PS(38:4) − H_8.806_811.5363 | 811.5363 |

### Classification and Machine Learning Approaches

Initial analysis performed on sets 1 and 2 evaluated random forests (Breiman, 2001), PLSDA using the caret R package implementation (Kuhn, 2020), logistic regression and support vector machines (R package e 1071 implementation) in the context of Sets 1 and 2; logistic regression was selected as giving best and more readily interpretable results using smaller lipid panels on the initial datasets.

Wherever lipid panels were evaluated, their classification potential was assessed using leave one out cross validation, where a model is trained on all but one sample, and the model performance is evaluated on the remaining sample. The process is repeated for all samples in turn, ensuring that at no point the data used for testing is also used for training. This approach was particularly relevant in the early stages where only a few datasets were available.

For each panel obtained via variable selection on one cohort or selection of sets, its classification success was assessed on the next available cohort.

As noted above, with the exception of refining the later small panels, the variable selection was carried out using Cohorts 1, 2 and 3 only, which in terms of cancer subcategories contained only invasive ductal carcinoma (IDC) samples This ensures that the results obtained on Cohort 4 show that the panels still have classification potential on datasets containing different early cancer subcategories (DCIS and ILC).

This sequential analysis path, combined with the different selection of methods for variable selection may explain, at least in part, the differences in lipid biomarkers identified by the statistical data analysis of Example 1 and the approach described for Example 2. Notwithstanding this, a number of lipid biomarkers (see overlapping panel of 6 biomarkers below) overlap between the diagnostic panels of Examples 1 and 2, which thereby demonstrates a high degree of consistency in identifying relevant lipid biomarkers by the two different methodologies.

### Panel Details

### Restricted Panel of 6 Markers-“Panel 6 FourMarkers”

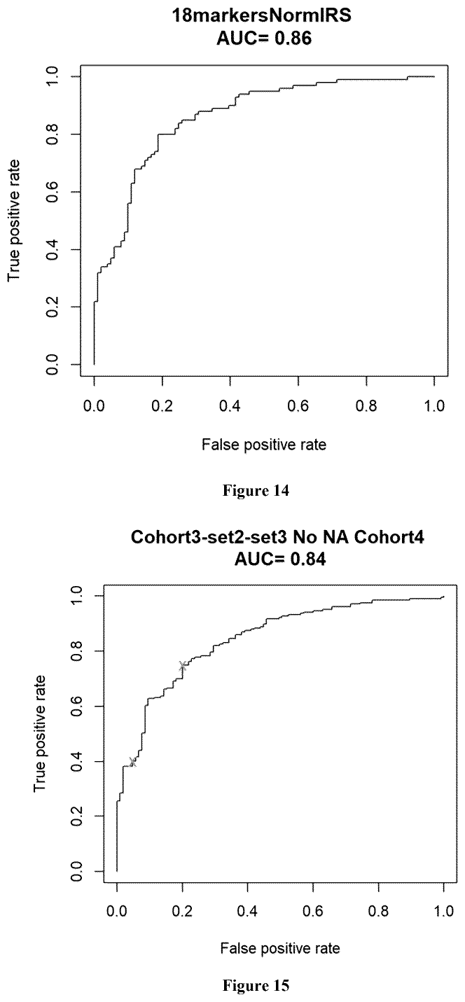

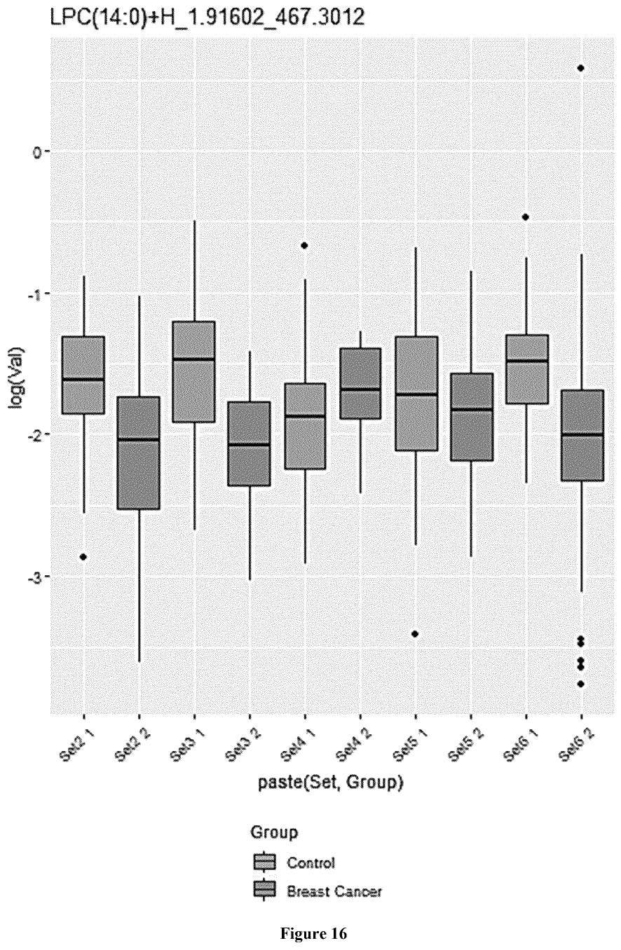

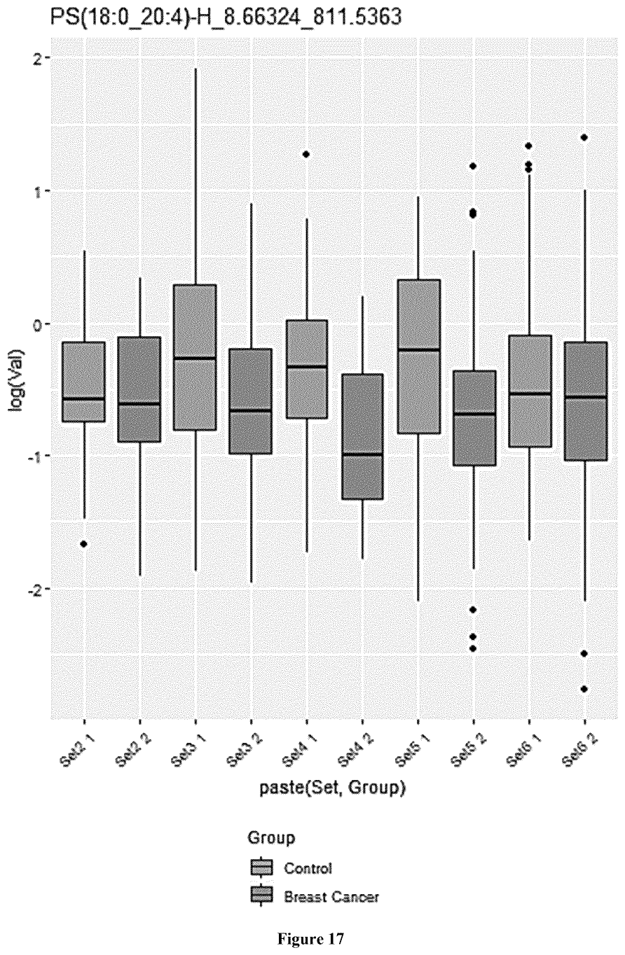

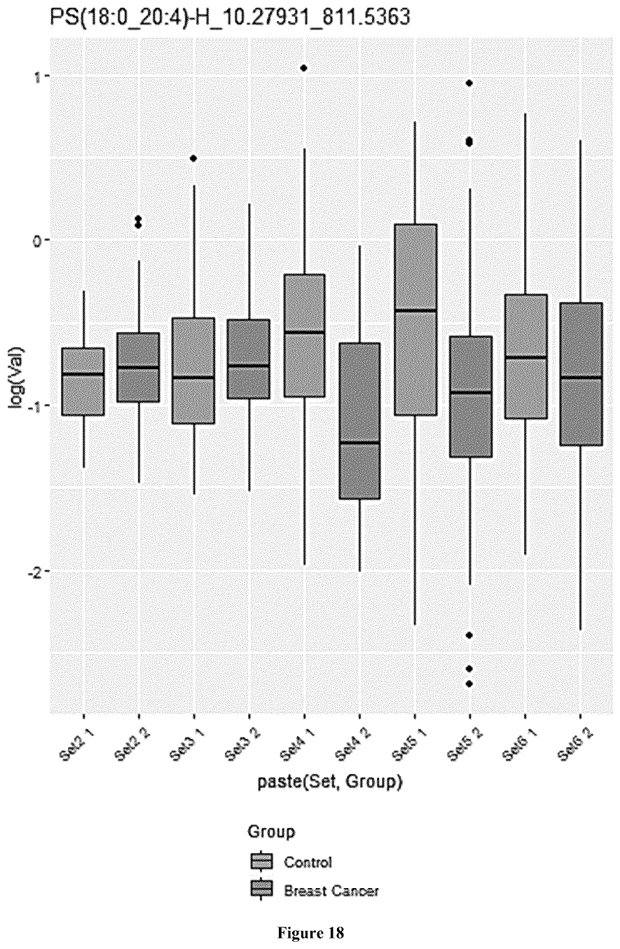

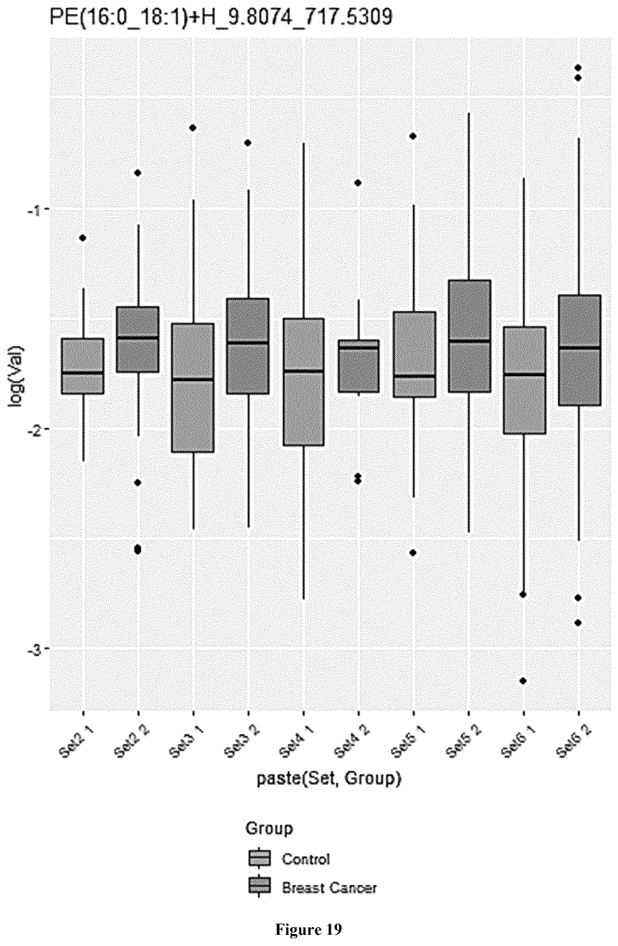

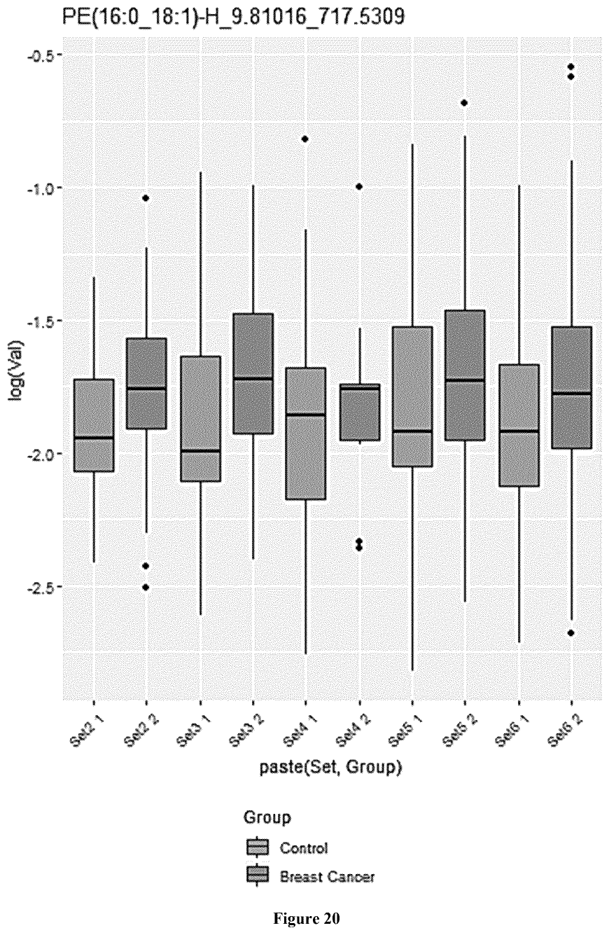

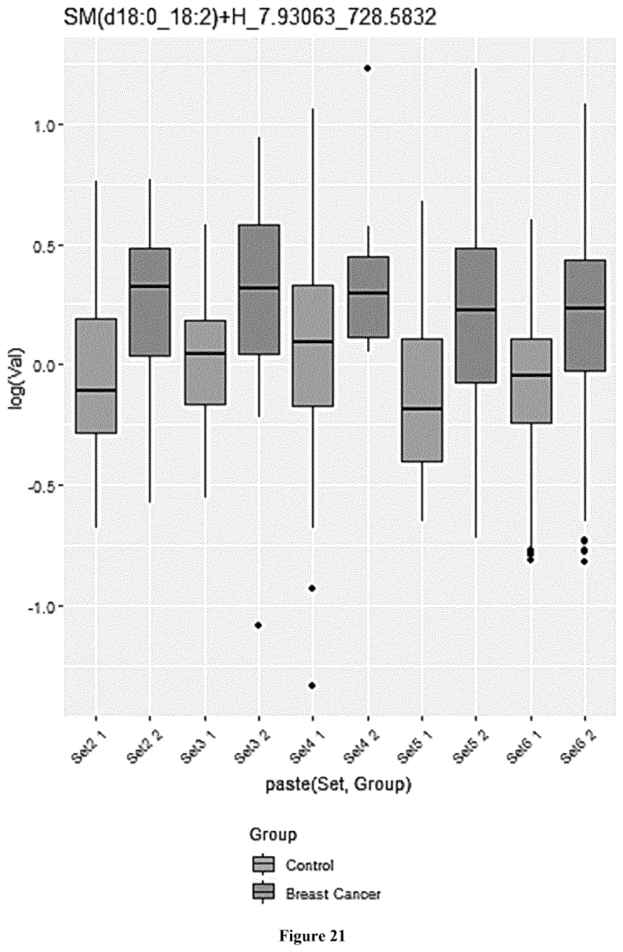

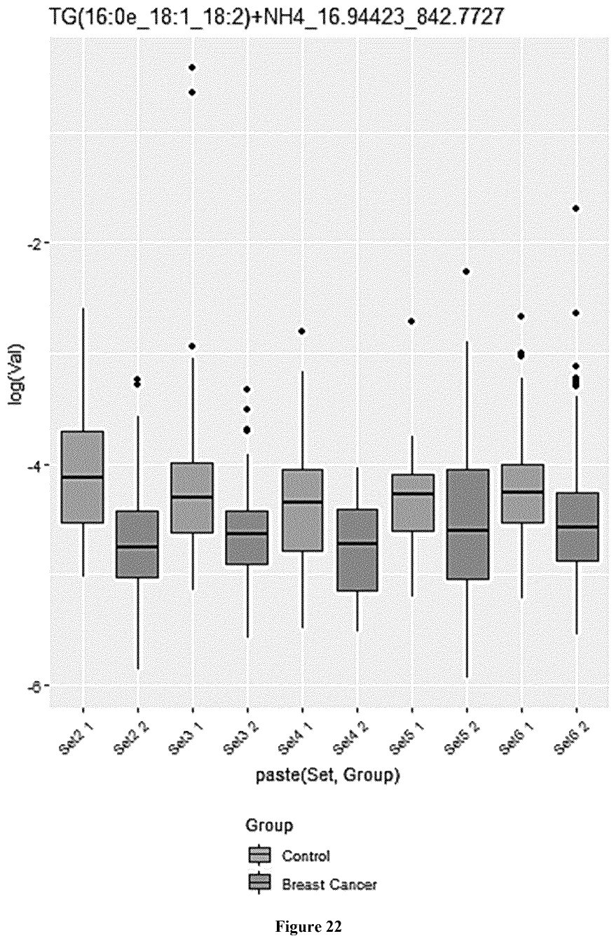

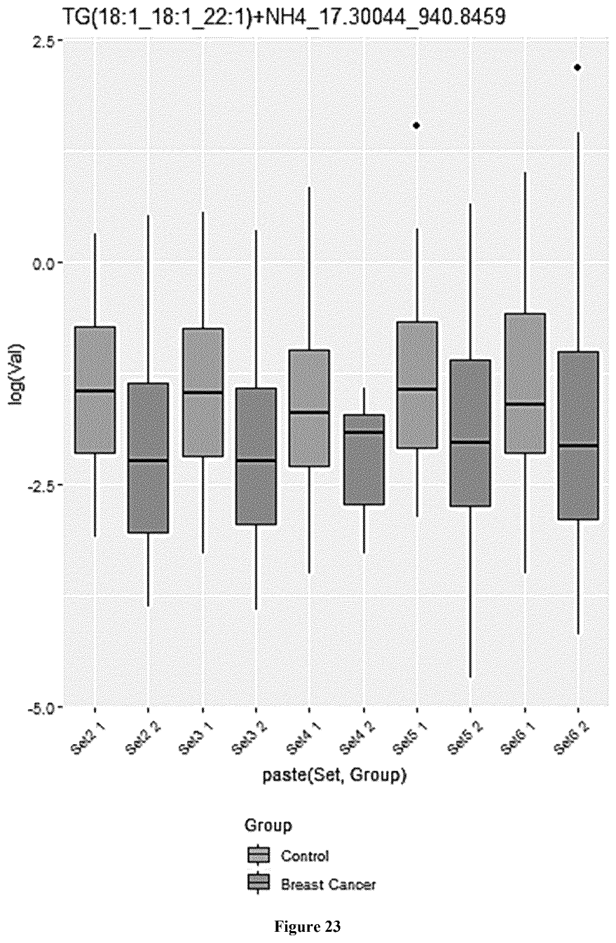

This panel contains two markers common to Example 1. As an extended example, the boxplots in FIGS. 16 to 23 show in detail the conserved pattern of expression or concentration over several available sets.

Panel of 6 Markers Common with the Validation Report “Common 6-ML_GBM”

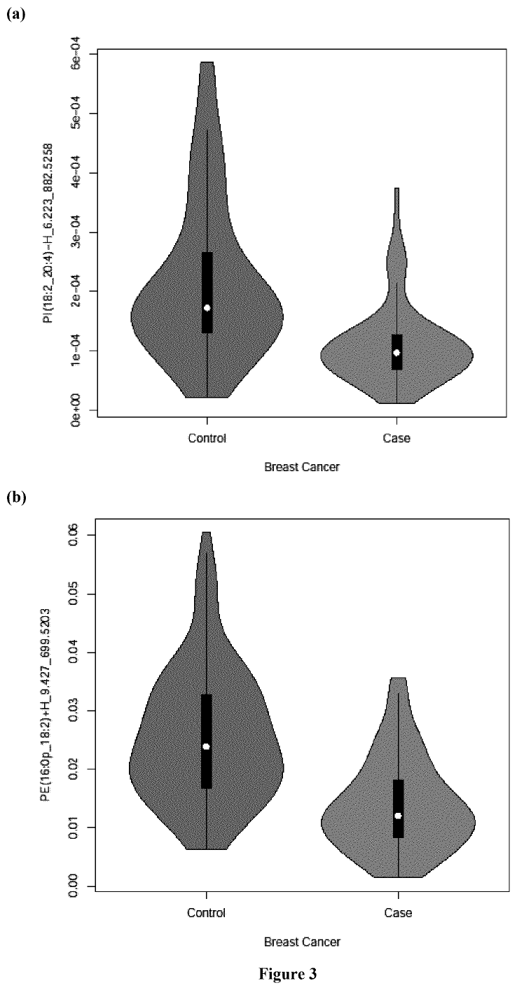

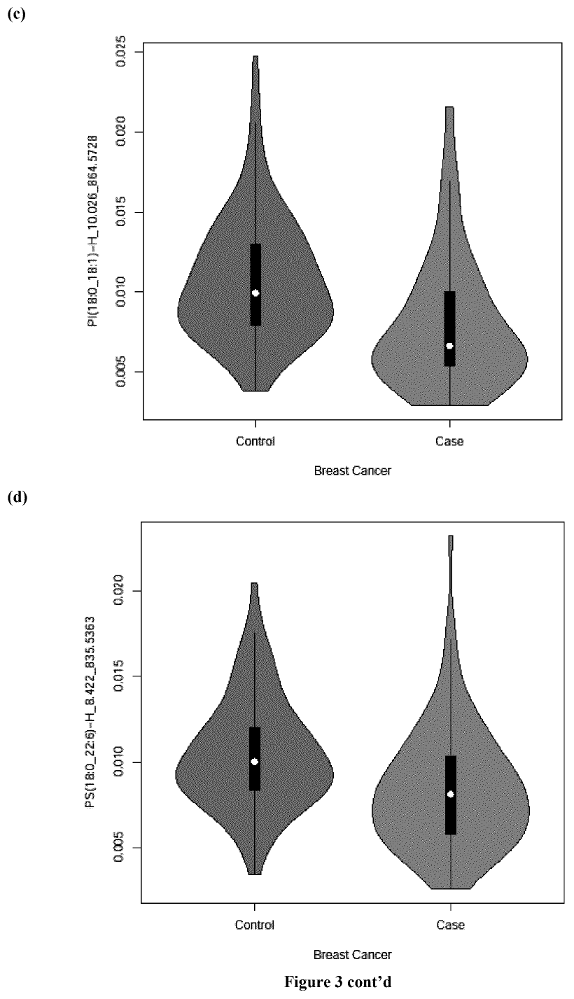

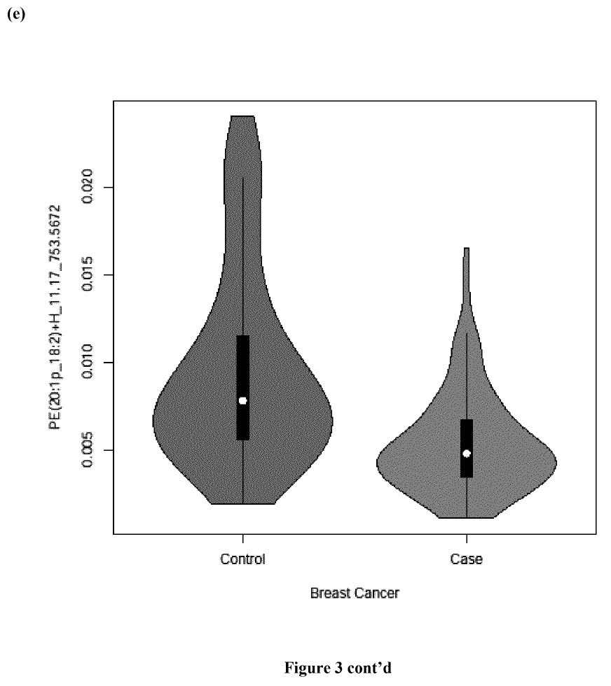

This panel is an important overlap panel, which contains all the lipid markers identified by both Examples 1 and 2 as being diagnostically relevant, and includes two of the m/z values in the previous panel of 6, as well as all the lipids from FIG. 3 (Cohort 4 volcano plot).

### ROC Curves

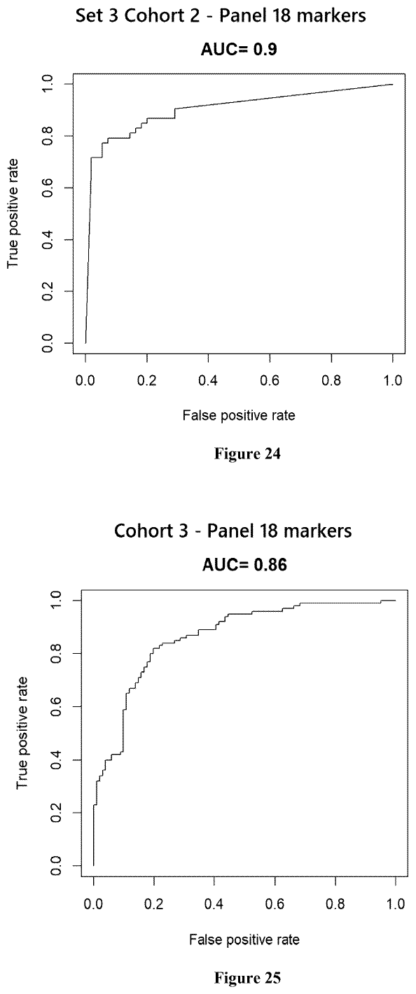

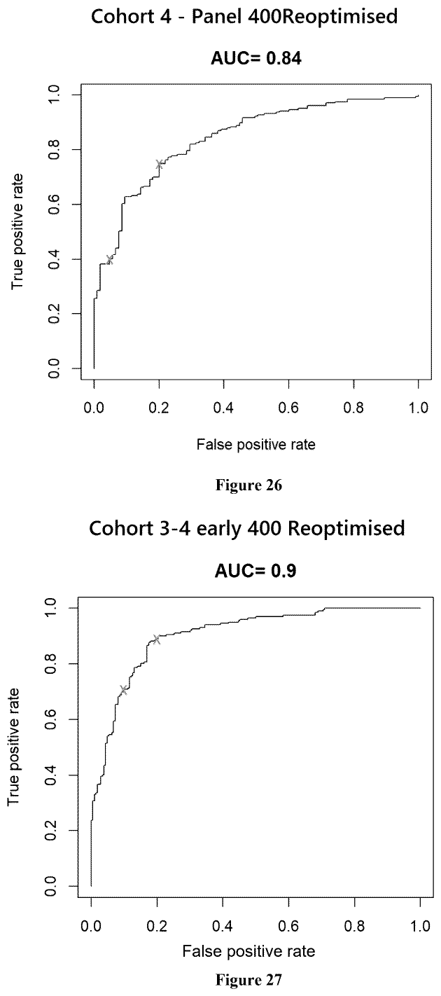

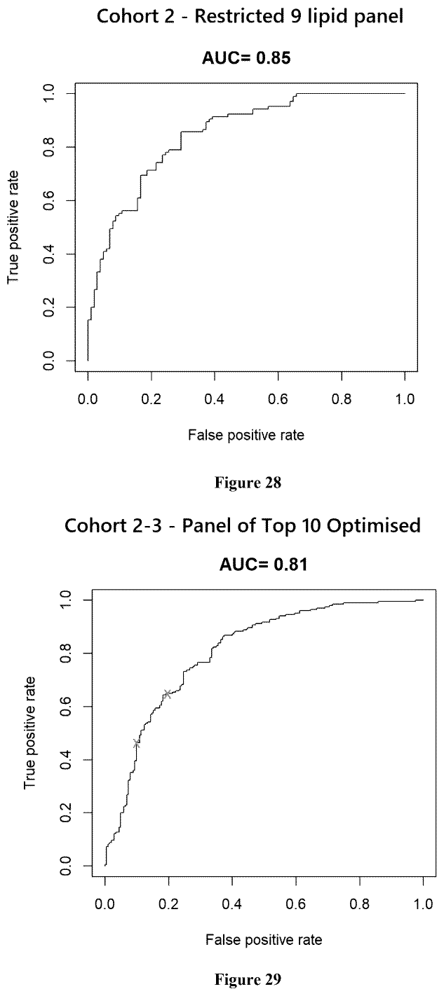

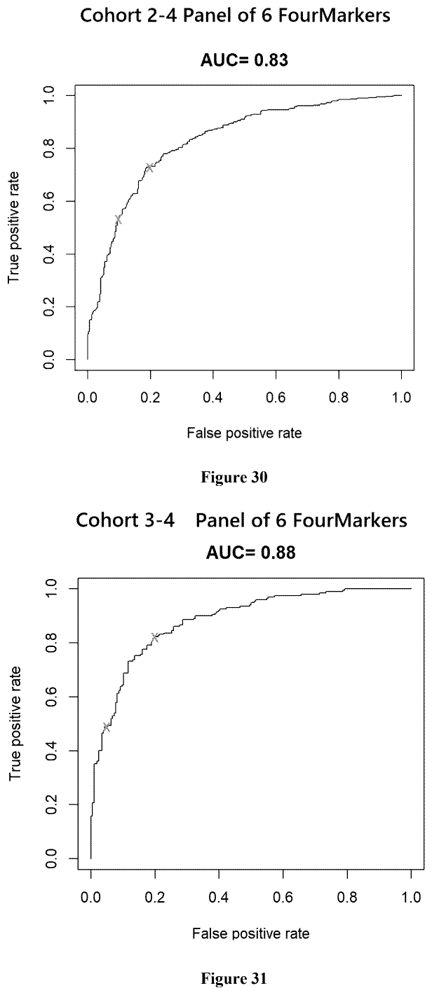

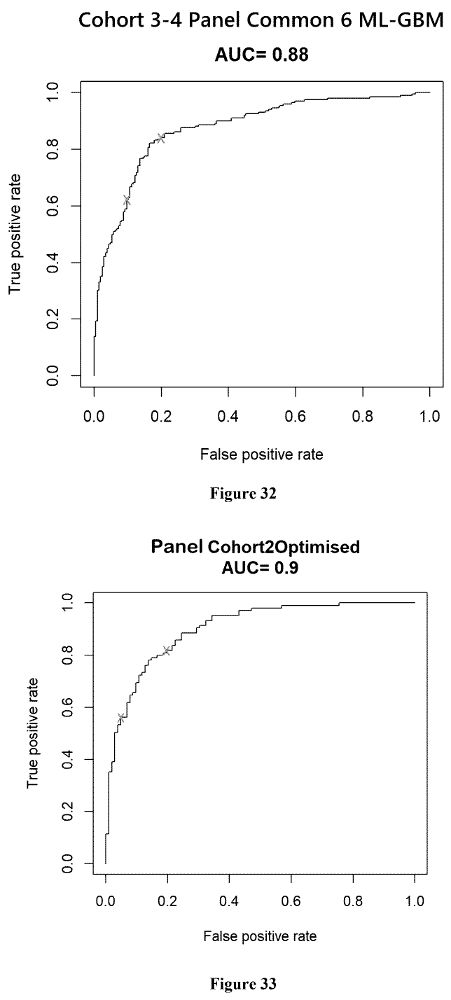

ROC curves for each of the panels from Example 2 are provided in FIGS. 14, 15 and 24 to 33. These curves demonstrate the diagnostic potential of each of the panels in patients with breast cancer, including early breast cancer. All curves were generated using leave one out cross validation.

### Example 3

Using Cohorts 3 and 4, the present inventors randomly drew m/z values (panels of either 6 or 18) to compare with the diagnostic value of the diagnostic panels identified in Examples 1 and 2. For the random panels of 6, 1000 simulations were run. For the random panel of 18, 100 simulations were run. We then tabulated the random panels that achieved a high random accuracy (defined as >75%), and compared their make-up to the earlier identified panels (see Tables 27 to 29 below).

### Results

The panels of 6 lipid biomarkers were amongst the best performers, with the ML_GBM from Table 23 being the best performer in its analysis (see Table 24), whilst the Panel of 6 FourMarkers from Table 22 was ranked third in its analysis (see Table 25). A high proportion of the top performing panels (random or otherwise) also contained the LPC(14:0) marker. A few of the random panels achieved a similar diagnostic performance.

When summarizing the most commonly occurring lipids from the well-performing random panels, three of the ML_GBM panel are in the top 5 (see Table 27).

TABLE 27
Analysis of 6 lipid biomarker panel ML_GBM

| Random | | | | | | | | |
|---|---|---|---|---|---|---|---|---|
| Run No. | RandomAccuracy | X1 | X2 | X3 | X4 | X5 | X6 | RunType |
| 1001 | 0.807018 | 467.3012 | 882.5258 | 716.5832 | 699.5203 | 811.5363 | 525.2855 | TRUE |
| 957 | 0.793372 | 517.3168 | 971.7273 | 467.3012 | 952.752 | 741.5672 | 795.5778 | RandomRun |
| 682 | 0.773879 | 954.7676 | 541.3168 | 924.7207 | 699.5203 | 567.3325 | 755.5829 | RandomRun |
| 116 | 0.766082 | 749.5359 | 859.6021 | 805.5622 | 784.6458 | 551.3951 | 904.752 | RandomRun |
| 627 | 0.766082 | 541.3168 | 781.5622 | 809.6745 | 779.5465 | 751.5516 | 862.7989 | RandomRun |
| 625 | 0.764133 | 777.5672 | 623.6216 | 798.6615 | 809.6745 | 809.5935 | 551.3951 | RandomRun |
| 424 | 0.762183 | 467.3012 | 896.7833 | 804.7207 | 743.5465 | 716.5832 | 799.6091 | RandomRun |
| 949 | 0.762183 | 750.6737 | 859.6021 | 737.4996 | 699.5203 | 797.6745 | 505.3532 | RandomRun |
| 817 | 0.756335 | 783.5778 | 796.6458 | 690.5676 | 772.6581 | 507.3325 | 467.3012 | RandomRun |
| 195 | 0.754386 | 858.5258 | 808.6458 | 910.5571 | 853.5622 | 749.5359 | 838.705 | RandomRun |
| 412 | 0.750487 | 795.5778 | 774.5676 | 753.5672 | 806.7363 | 926.7363 | 785.5935 | RandomRun |
| 830 | 0.750487 | 747.5778 | 900.7207 | 771.5778 | 467.3012 | 767.5465 | 791.5465 | RandomRun |
| 850 | 0.750487 | 805.5622 | 757.5622 | 807.5778 | 904.752 | 781.5622 | 699.5203 | RandomRun |

TABLE 28
Analysis of 6 lipid biomarker panel “Panel of 6 FourMarkers”

| Random | | | | | | | | |
|---|---|---|---|---|---|---|---|---|
| Run No. | Accuracy | MZ1 | MZ2 | MZ3 | MZ4 | MZ5 | MZ6 | RunType |
| 700 | 0.80117 | 751.5516 | 802.6928 | 743.5465 | 467.3012 | 834.5258 | 836.6771 | Random Run |
| 144 | 0.783626 | 674.5363 | 624.5845 | 467.3012 | 816.7084 | 777.5672 | 936.8146 | RandomRun |
| 1001 | 0.777778 | 717.5309 | 811.5363 | 467.3012 | 728.5832 | 842.7727 | 940.8459 | TRUE |
| 155 | 0.775828 | 543.3325 | 775.5516 | 620.538 | 467.3012 | 783.5778 | 826.6928 | RandomRun |
| 529 | 0.764133 | 914.5884 | 831.5778 | 467.3012 | 820.752 | 543.3325 | 605.442 | RandomRun |
| 415 | 0.762183 | 789.6248 | 932.7833 | 541.3168 | 820.752 | 727.5516 | 812.6771 | RandomRun |
| 220 | 0.760234 | 771.5778 | 467.3012 | 791.5829 | 567.3325 | 774.5676 | 835.5363 | RandomRun |
| 447 | 0.760234 | 861.6177 | 840.7207 | 565.5434 | 777.5672 | 828.7084 | 898.7989 | RandomRun |
| 13 | 0.750487 | 467.3012 | 936.8146 | 501.2855 | 746.6424 | 781.5622 | 784.6458 | Random Run |
| 73 | 0.750487 | 796.6458 | 811.6091 | 910.7989 | 739.5152 | 781.5622 | 751.5516 | RandomRun |
| 627 | 0.750487 | 537.3794 | 569.3481 | 808.6458 | 811.5363 | 781.5622 | 727.5516 | RandomRun |
| 963 | 0.750487 | 807.5778 | 844.752 | 547.3638 | 715.5152 | 782.6302 | 878.7363 | RandomRun |

TABLE 29
Analysis of BCAL current panel of 18 400 Reoptimised from Table 19

| Random | Random | | | | | | | | | | |
|---|---|---|---|---|---|---|---|---|---|---|---|
| Run no. | Accuracy | X1 | X2 | X3 | X4 | X5 | X6 | X7 | X8 | X9 | X10 |
| 28 | 0.769981 | 852.7207 | 783.5778 | 535.3638 | 811.5363 | 882.5258 | 803.5465 | 620.2962 | 739.5152 | 819.6142 | 507.3325 |
| 52 | 0.764133 | 914.5884 | 757.5985 | 826.705 | 878.7363 | 834.7676 | 770.6302 | 696.5206 | 876.7207 | 818.7727 | 730.5989 |
| 29 | 0.762183 | 902.7363 | 787.6091 | 819.6142 | 607.4577 | 767.5829 | 727.5516 | 809.5935 | 906.7676 | 719.5465 | 507.3325 |
| 54 | 0.756335 | 765.5309 | 860.7833 | 805.5622 | 699.5203 | 898.7989 | 748.6581 | 719.5829 | 777.5672 | 890.5884 | 745.5985 |
| 35 | 0.754386 | 890.5884 | 569.3481 | 800.6771 | 767.5829 | 479.3376 | 727.5516 | 831.5778 | 765.5309 | 765.5672 | 579.4264 |
| 73 | 0.754386 | 803.5465 | 998.9241 | 844.752 | 493.3168 | 769.5985 | 918.8615 | 747.5778 | 888.5728 | 805.5622 | 882.7676 |
| 7 | 0.750487 | 842.7727 | 771.5778 | 876.7207 | 688.5519 | 769.5985 | 521.3481 | 607.4577 | 798.6615 | 481.3532 | 864.5728 |
| 34 | 0.750487 | 743.5829 | 861.6177 | 800.6771 | 837.6248 | 812.6771 | 912.5728 | 814.6928 | 782.6302 | 743.5465 | 826.6928 |
| [symbol] | [symbol] | [symbol] | [symbol] | [symbol] | [symbol] | [symbol] | [symbol] | [symbol] | [symbol] | [symbol] | [symbol] |

| Random | | | | | | | | | |
|---|---|---|---|---|---|---|---|---|---|
| Run no. | X11 | X12 | X13 | X14 | X15 | X16 | X17 | X18 | RunType |
| 28 | 861.6177 | 812.6771 | 751.5516 | 716.5832 | 773.5935 | 722.5363 | 830.7363 | 837.6248 | RandomRun |
| 52 | 882.7676 | 741.5672 | 537.3794 | 852.7207 | 862.5571 | 537.3794 | 773.5935 | 811.5363 | RandomRun |
| 29 | 880.752 | 751.5516 | 747.5203 | 571.3638 | 805.5622 | 453.2855 | 850.705 | 820.6458 | RandomRun |
| 54 | 809.6745 | 924.7207 | 715.5152 | 813.6248 | 783.5778 | 725.5359 | 751.5516 | 644.4893 | RandomRun |
| 35 | 842.7727 | 704.5832 | 793.5985 | 757.5622 | 788.6894 | 765.5672 | 802.705 | 882.7676 | RandomRun |
| 73 | 882.5258 | 622.5689 | 467.3012 | 481.3168 | 688.5519 | 674.5363 | 749.5359 | 721.5046 | RandomRun |
| 7 | 830.7363 | 817.5985 | 528.7207 | 770.6302 | 549.3794 | 809.5935 | 829.5622 | 910.7989 | RandomRun |
| 34 | 694.6111 | 507.3325 | 788.6771 | 715.5152 | 858.7676 | 826.705 | 727.5516 | 621.606 | RandomRun |
| [symbol] | [symbol] | [symbol] | [symbol] | [symbol] | [symbol] | [symbol] | [symbol] | [symbol] | [symbol] |

[symbol]

indicates data missing or illegible when filed

TABLE 30
Frequency of lipid biomarkers appearing in random runs

| | Number of times it occurred in |
|---|---|
| MZ | high performing draws |
| 467.3012 | 14 |
| 751.5516 | 6 |
| 811.5363 | 6 |
| 699.5203 | 5 |
| 727.5516 | 5 |
| 781.5622 | 5 |
| 805.5622 | 5 |
| 507.3325 | 4 |
| 777.5672 | 4 |
| 783.5778 | 4 |
| 842.7727 | 4 |
| 882.7676 | 4 |
| 537.3794 | 3 |
| 541.3168 | 3 |
| 715.5152 | 3 |
| 716.5832 | 3 |
| 743.5465 | 3 |
| 749.5359 | 3 |
| 771.5778 | 3 |
| 807.5778 | 3 |
| 809.5935 | 3 |
| 809.6745 | 3 |
| 812.6771 | 3 |
| 861.6177 | 3 |
| 878.7363 | 3 |
| 882.5258 | 3 |
| 543.3325 | 2 |
| 551.3951 | 2 |
| 567.3325 | 2 |
| 569.3481 | 2 |
| 607.4577 | 2 |
| 674.5363 | 2 |
| 688.5519 | 2 |
| 717.5309 | 2 |
| 728.5832 | 2 |
| 739.5152 | 2 |
| 741.5672 | 2 |
| 747.5778 | 2 |
| 757.5622 | 2 |
| 765.5309 | 2 |
| 765.5672 | 2 |
| 767.5829 | 2 |
| 769.5985 | 2 |
| 770.6302 | 2 |
| 773.5935 | 2 |
| 774.5676 | 2 |
| 782.6302 | 2 |
| 784.6458 | 2 |
| 785.5935 | 2 |
| 795.5778 | 2 |
| 796.6458 | 2 |
| 798.6615 | 2 |
| 800.6771 | 2 |
| 803.5465 | 2 |
| 808.6458 | 2 |
| 819.6142 | 2 |
| 820.752 | 2 |
| 826.6928 | 2 |
| 826.705 | 2 |
| 830.7363 | 2 |
| 831.5778 | 2 |
| 836.6771 | 2 |
| 837.6248 | 2 |
| 844.752 | 2 |
| 852.7207 | 2 |
| 859.6021 | 2 |
| 876.7207 | 2 |
| 880.752 | 2 |
| 890.5884 | 2 |
| 898.7989 | 2 |
| 904.752 | 2 |
| 910.7989 | 2 |
| 914.5884 | 2 |
| 924.7207 | 2 |
| 936.8146 | 2 |
| 940.8459 | 2 |
| 453.2855 | 1 |
| 479.3376 | 1 |
| 481.3168 | 1 |
| 481.3532 | 1 |
| 493.3168 | 1 |
| 501.2855 | 1 |
| 505.3532 | 1 |
| 517.3168 | 1 |
| 521.3481 | 1 |
| 525.2855 | 1 |
| 535.3638 | 1 |
| 547.3638 | 1 |
| 549.3794 | 1 |
| 565.5434 | 1 |
| 571.3638 | 1 |
| 579.4264 | 1 |
| 605.442 | 1 |
| 620.2962 | 1 |
| 620.538 | 1 |
| 621.606 | 1 |
| 622.5689 | 1 |
| 623.6216 | 1 |
| 624.5845 | 1 |

### Example 4

In the present Example, the inventors conducted an experiment to examine whether fed extracellular vesicle (EV) samples can be used to detect breast cancer alongside fasted EV samples, as well as whether plasma samples, fasted or fed, can be used directly to detect breast cancer. The inventors also aimed to establish whether any of their existing lipid signatures determined using EV fasted samples from various past cohorts could be used to assess fed EV samples, or fasted plasma samples.

### Study Design

Lipids in fasted and non-fasted EV and plasma samples were extracted from 60 control patients. The matched samples were analyzed in two separate labs, internally at BCAL (n=26 of the total 60 samples), and externally at the Baker Heart and Diabetes Institute (n=60 samples). The BCAL lab samples contained the full complement of lipids previously measured in past cohorts and hence in previous lipid signatures of interest. The Baker lab samples did not identify the full complement of BCAL lipids, however assessed a larger number of patient samples.

The resulting lipid quantification was tested against the existing BCAL signatures wherever possible, and correlations of classification scores obtained between matched fasted and fed samples were reported.

### Data Analysis

Identification of Lipids that Change with Fasted Fed Status

The matched control samples were used to find which lipids change in concentration between fasted and fed states in EV samples and separately in plasma, and similarly between plasma and EV samples.

In terms of data processing, lipids with missing values were removed. The data was shifted by a small value eps=0.001 to allow for log transform of zero values. A two-tailed paired t-test was applied separately for all lipids, and any lipid was regarded as significantly different if the FDR-adjusted (Benjamini and Hochberg) p-value was less than 0.05.

Identify Lipid Signatures Showing Robust Results with Fasted or Fed Samples

The present lipid signatures were examined to see if the concentrations of the lipids change depending on fasted/fed status: it was conjectured that lipid signatures which do not change upon feeding could potentially be used to classify fed samples as well. The lipids panels investigated were the 400 Reoptimised lipid panel of 18, Panel of 6-Four Markers and Common 6-ML_GBM panels.

For each of the lipid signatures considered (typically trained using Cohorts 2-3), the model prediction script was run to normalise the new data, apply the signature and generate cancer probabilities for each of the samples.

The fed/fasted samples were matched side by side, and the resulting scores were compared, generating fit lines and statistics (fit line coefficients, R-squared, correlation, correlation significance).

### Results

Differential Abundance with Fasting Status

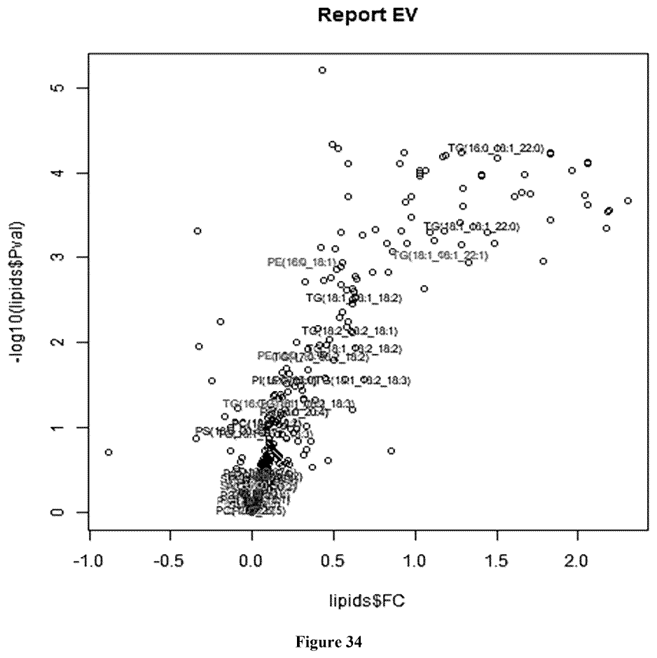

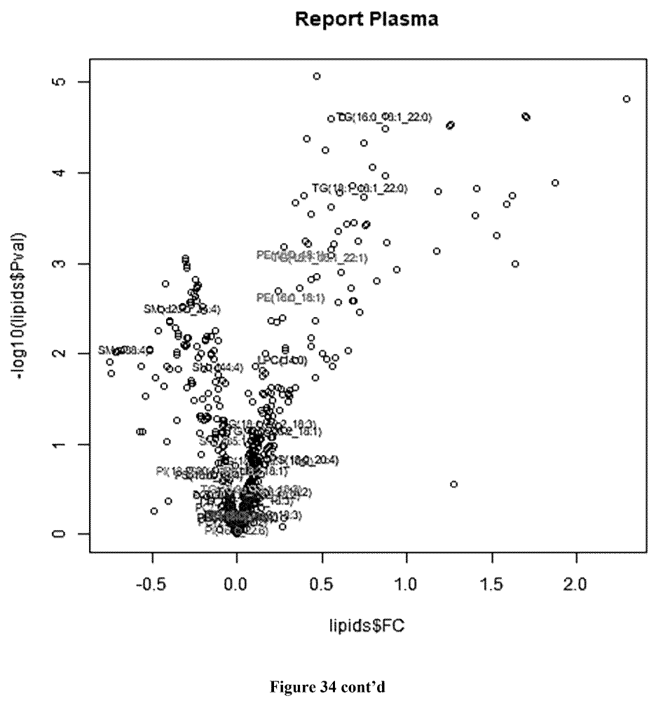

Statistically significant differences were not observed in the concentration of lipids in the BCAL panel Common 6-ML_GBM between fed and fasted states in either EV or plasma samples (FIG. 34 and Table 31). The other two panels contained several lipids which showed significant differences in their concentrations between fed and fasted states. Table 31 details all panel m/z values of the lipids, of which the concentrations changed with fasted/fed status.

TABLE 31
Signature Lipids changing in abundance with fast/fed status; FALSE
means no significant change observed. None of the lipids present in the
ML_GBM panel change with fasting status in either EV or plasma.

| | | | Differentially expressed | Differentially expressed with |
|---|---|---|---|---|
| | Panel | M/z | with fast/fed status EV | fast/fed status plasma |
| 1 | 400Reoptimised | 467.3012 | FALSE | FALSE |
| 2 | 400Reoptimised | 731.5465 | FALSE | FALSE |
| 3 | 400Reoptimised | 807.5778 | FALSE | FALSE |
| 4 | 400Reoptimised | 717.5309 | TRUE | TRUE |
| 5 | 400Reoptimised | 836.5415 | FALSE | FALSE |
| 6 | 400Reoptimised | 811.5363 | FALSE | FALSE |
| 7 | 400Reoptimised | 728.5832 | FALSE | FALSE |
| 8 | 400Reoptimised | 752.5832 | FALSE | FALSE |
| 9 | 400Reoptimised | 836.6771 | FALSE | TRUE |
| 10 | 400Reoptimised | 842.7727 | FALSE | FALSE |
| 11 | 400Reoptimised | 882.7676 | TRUE | FALSE |
| 12 | 400Reoptimised | 880.7520 | TRUE | FALSE |
| 13 | 400Reoptimised | 916.8459 | TRUE | TRUE |
| 14 | 400Reoptimised | 942.8615 | TRUE | TRUE |
| 15 | 400Reoptimised | 940.8459 | TRUE | TRUE |
| 16 | 400Reoptimised | 785.5935 | FALSE | FALSE |
| 17 | 400Reoptimised | 868.7520 | FALSE | FALSE |
| 18 | 400Reoptimised | 878.7363 | FALSE | FALSE |
| 19 | Panel of 6 FourMarkers | 717.5309 | TRUE | TRUE |
| 20 | Panel of 6 FourMarkers | 811.5363 | FALSE | FALSE |
| 21 | Panel of 6 FourMarkers | 467.3012 | FALSE | FALSE |
| 22 | Panel of 6 FourMarkers | 728.5832 | FALSE | FALSE |
| 23 | Panel of 6 FourMarkers | 842.7727 | FALSE | FALSE |
| 24 | Panel of 6 FourMarkers | 940.8459 | TRUE | TRUE |
| 25 | ML_GBM | 467.3012 | FALSE | FALSE |
| 26 | ML_GBM | 882.5258 | FALSE | FALSE |
| 27 | ML_GBM | 716.5832 | FALSE | FALSE |
| 28 | ML_GBM | 699.5203 | FALSE | FALSE |
| 29 | ML_GBM | 811.5363 | FALSE | FALSE |
| 30 | ML_GBM | 525.2855 | FALSE | FALSE |

Correlation of Lipid Signature Scores for BCAL Dataset (n=26)

Table 32 summarises the score alignment fit for the signatures of interest. Common 6-ML_GBM yields the best scores for correlations between fasted and fed matched EV samples, second only to the baseline example consisting of frozen repeat samples previously analyzed by BCAL. The fasted EV and plasma samples are also highly correlated.

TABLE 32
Correlation of classification scores obtained with various lipid signatures
for the samples analyzed (n = 26 control samples); panels containing
fewer markers changing with fasting status yield better scores correlations

| | | Pearson | Correlation | Number DE | |
|---|---|---|---|---|---|
| Panel used | R-Squared | Correlation | p-value | lipids in panel | SET |
| ML_GBM | 86% | 93% | 1.00E−19 | NA | Baseline: |
| | | | | | frozen repeats |
| ML_GBM | 63% | 79% | 1.37E−06 | none | EV fed vs |
| | | | | | fasted |
| ML_GBM | 52% | 72% | 3.00E−05 | NA | Fasted Evs vs |
| | | | | | fasted Plasma |
| Panel of 6 | 50% | 71% | 5.32E−05 | NA | Fasted Evs vs |
| FourMarkers | | | | | fasted Plasma |
| Panel of 6 | 44% | 66% | 2.23E−04 | 2 out of 6 | EV fed vs |
| FourMarkers | | | | | fasted |
| ML_GBM | 39% | 62% | 6.88E−04 | none | Plasma fed vs |
| | | | | | fasted |
| 400Reoptimised | 35% | 59% | 1.45E−03 | NA | Fasted Evs vs |
| | | | | | fasted Plasma |
| Panel of 6 | 32% | 56% | 2.85E−03 | 2 out of 6 | Plasma fed vs |
| FourMarkers | | | | | fasted |
| 400Reoptimised | 29% | 54% | 4.78E−03 | 5 out of 18 | Plasma fed vs |
| | | | | | fasted |
| 400Reoptimised | 23% | 48% | 1.40E−02 | 6 out of 18 | EV fed vs |
| | | | | | fasted |

Similarity of Lipid Signature Scores for External Laboratory Dataset (n=60)

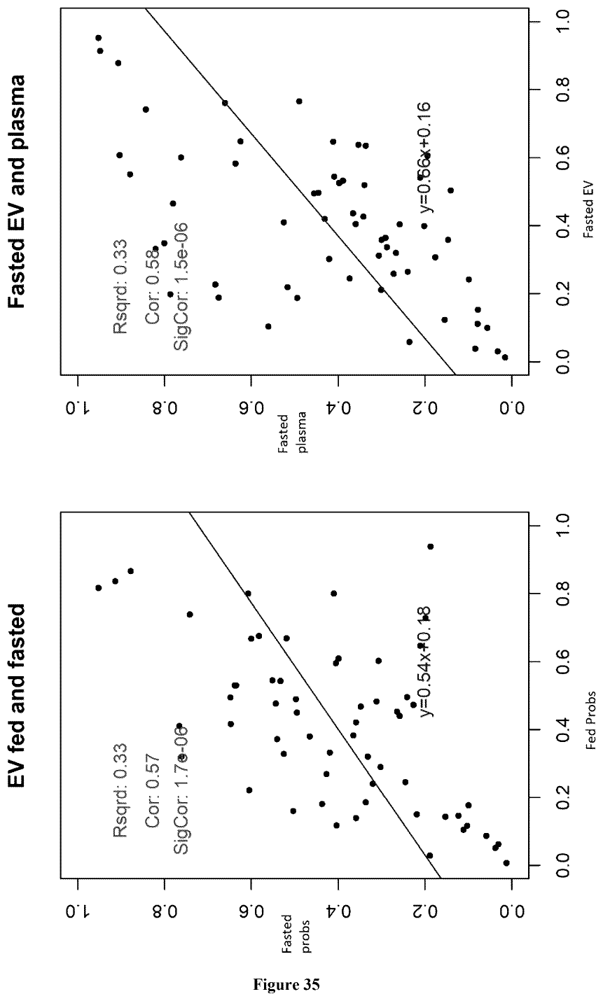

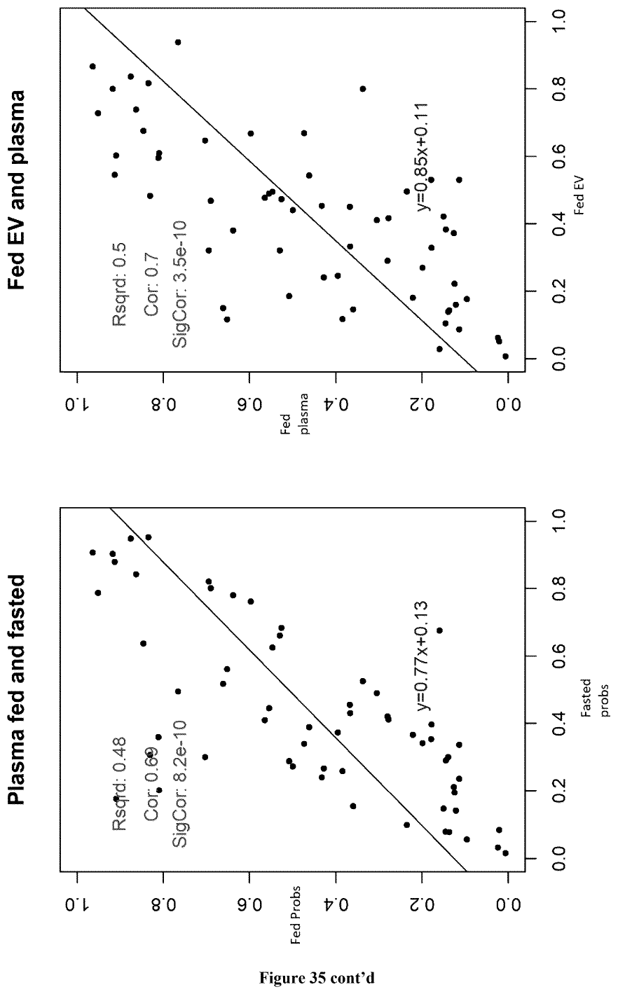

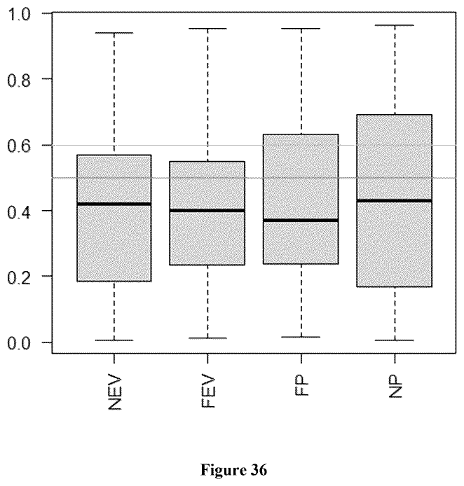

Although not all lipids from the BCAL signatures were included in this analysis, all 6 lipids from the Common 6-ML_GBM could be matched between both datasets; FIG. 35 and Table 33 below summarizes the score alignment obtained with this signature on the new dataset (n=60 controls). The matched scores obtained were significantly correlated. As all samples were controls, samples with low cancer probability scores represent a correct classification (FIG. 36). For both fasted samples and non-fasted samples, the median score was less than 0.5 while for fasted and non-fasted EV samples more than 75% of the samples have model probability <0.6.

TABLE 33
Correlation of scores obtained with ML_GBM panel for samples
analyzed on new dataset (n = 60 control samples)

| Signature | | Pearson | Correlation | |
|---|---|---|---|---|
| used | R-Squared | Correlation | P-value | SET |
| ML_GBM | 0.50 | 0.70 | 3.48E−10 | Fed EV and |
| | | | | plasma |
| ML_GBM | 0.48 | 0.69 | 8.19E−10 | Plasma fed and |
| | | | | fasted |
| ML_GBM | 0.33 | 0.58 | 1.50E−06 | Fasted EV and |
| | | | | plasma |
| ML_GBM | 0.33 | 0.57 | 1.73E−06 | EV fed and |
| | | | | fasted |

### CONCLUSION

The experiments conducted show that the lipid biomarker panels described herein (e.g., Common 6-ML_GBM) may not contain any lipids that change significantly in concentration between fed and fasted states. Hence, the Common 6-ML_GBM lipid signature could potentially be used to assess non-fasted patient EV samples as well as fasted EV samples. The classification algorithm scores generated on the new test data are significantly correlated between patient-matched fasted and non-fasted EV samples, as well as between fasted plasma and fasted EV samples, both in the data generated internally by BCAL, and on the external laboratory data.

### REFERENCES

Chen T, He T, Benesty M et al (2021). xgboost: Extreme Gradient Boosting. R package version 1.4.1.1. https://CRAN.R-project.org/package=xgboost
Friedman J H (2001). Greedy function approximation: a gradient boosting machine. Ann Statist 29 (5): 1189-1232.
Friedman J H (2002). Stochastic gradient boosting. Comput Stat Data Anal 38 (4): 367-378.
Johnson W E, Li C, Rabinovic A (2007). Adjusting batch effects in microarray expression data using empirical Bayes methods. Biostatistics 8(1):118-27.
Mistry D A H, French P W (2016). Circulating phospholipids as biomarkers of breast cancer: a review. Breast Cancer: Basic and Clinical Research 10:191-196.
Society A C. Cancer Facts and Figures 2016. Atlanta: American Cancer Society, Inc. Available at: http://www.cancer.org/cancer/breastcancerinmen/detailedguide/breast-cancer-in-men-key-statistics

1. A method of diagnosing a subject with a breast cancer, said method including the step of measuring a level of one or more lipid biomarkers in a biological sample from the subject, wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof, and wherein the level of the one or more lipid biomarkers is diagnostic or indicative of the subject having the breast cancer.

2. The method of claim 1, wherein an increased level of Cer(d36:1), Cer(d42:0), DG(34:2), Hex2Cer(d34:1), Hex2Cer(d42:2), LPE(22:6), LPI(20:4), PC(32:1), PC(34:1), PC(35:4), PC(36:2), PC(36:3), PC(37:4), PC(38:5), PE(34:1), PI(32:1), PI(34:1), PS(38:4), PS(40:6), SM(d32:2), SM(d33:1), SM(d35:1), SM(d36:1), SM(d36:2), SM(d38:4), SM(d40:3), SM(d41:2), SM(d41:3), SM(d42:1), SM(d42:4) and/or SM(d44:4), or a fragment, variant or derivative thereof, and/or a decreased level of Cer(42:1), LPC(14:0), LPC(16:0e), LPC(17:1), LPC(18:3), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and/or TG(62:2), or a fragment, variant or derivative thereof, is diagnostic or indicative of the subject having the breast cancer.

3. The method of claim 1 or claim 2, further including the step of administering a treatment for the breast cancer to the subject.

4. A method for measuring a level of one or more lipid biomarkers in a biological sample from a subject, said method including the steps of:
(a) providing the biological sample; and
(b) measuring the level of the one or more lipid biomarkers in the biological sample, wherein the one or more lipid biomarkers are selected from the group consisting of Cer(d36:1), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

5. The method of claim 4, wherein the subject is suspected of having a breast cancer or has been previously diagnosed with a breast cancer.

6. The method of claim 4 or claim 5, wherein the measuring step includes determining the presence or absence of: (i) an increased level of Cer(d36:1), Cer(d42:0), DG(34:2), Hex2Cer(d34:1), Hex2Cer(d42:2), LPE(22:6), LPI(20:4), PC(32:1), PC(34:1), PC(35:4), PC(36:2), PC(36:3), PC(37:4), PC(38:5), PE(34:1), PI(32:1), PI(34:1), PS(38:4), PS(40:6), SM(d32:2), SM(d33:1), SM(d35:1), SM(d36:1), SM(d36:2), SM(d38:4), SM(d40:3), SM(d41:2), SM(d41:3), SM(d42:1), SM(d42:4) and/or SM(d44:4), or a fragment, variant or derivative thereof, and/or (ii) a decreased level of Cer(42:1), LPC(14:0), LPC(16:0e), LPC(17:1), LPC(18:3), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and/or TG(62:2), or a fragment, variant or derivative thereof, in the biological sample of the subject.

7. A method of treating a breast cancer in a subject, said method including the step of performing a treatment in respect of the subject in which a level of one or more lipid biomarkers has been measured in a biological sample therefrom that is diagnostic or indicative of the subject having the breast cancer, wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

8. The method of claim 7, wherein the treatment includes administering a therapeutically effective amount of an anti-cancer treatment to the subject.

9. The method of claim 7 or claim 8, in which an increased level of Cer(d36:1), Cer(d42:0), DG(34:2), Hex2Cer(d34:1), Hex2Cer(d42:2), LPE(22:6), LPI(20:4), PC(32:1), PC(34:1), PC(35:4), PC(36:2), PC(36:3), PC(37:4), PC(38:5), PE(34:1), PI(32:1), PI(34:1), PS(38:4), PS(40:6), SM(d32:2), SM(d33:1), SM(d35:1), SM(d36:1), SM(d36:2), SM(d38:4), SM(d40:3), SM(d41:2), SM(d41:3), SM(d42:1), SM(d42:4) and/or SM(d44:4), or a fragment, variant or derivative thereof, and/or a decreased level of Cer(42:1), LPC(14:0), LPC(16:0e), LPC(17:1), LPC(18:3), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and/or TG(62:2), or a fragment, variant or derivative thereof, was measured from the biological sample of the subject.

10. The method of any one of the preceding claims, wherein the one or more lipid biomarkers comprise LPC(14:0), or a fragment, variant or derivative thereof, and one or more other lipid biomarkers selected from the group consisting of Cer(d36:1), Cer(d42:0), Cer(d42:1), DG(34:2), Hex2Cer(d34:1), Hex2Cer(d42:2), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

11. The method of any one of the preceding claims, wherein the one or more lipid biomarkers comprise PI(38:6), or a fragment, variant or derivative thereof, and one or more other lipid biomarkers selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

12. The method of any one of the preceding claims, wherein the one or more lipid biomarkers comprise LPC(14:0) and PI(38:6), or a fragment, variant or derivative thereof, and optionally one or more other lipid biomarkers selected from the group consisting of Cer(d36:1), Cer(d42:0), Cer(d42:1), DG(34:2), Hex2Cer(d34:1), Hex2Cer(d42:2), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

13. The method of any one of claims 1 to 10, wherein the one or more lipid biomarkers are selected from the group consisting of LPC(14:0), PC(32:1), PC(38:5), PE(34:1), PS(38:4), SM(d36:2), SM(d38:4), TG(54:4), TG(56:1) and TG(58:2), or a fragment, variant or derivative thereof.

14. The method of any one of claims 1 to 10, wherein the one or more lipid biomarkers are selected from the group consisting of LPC(14:0), PC(32:1), PC(36:2), PC(38:5), PE(34:1), PI(34:1), PS(38:4), SM(d36:2), SM(d38:4), SM(d44:4), TG(52:3e), TG(53:4), TG(54:4), TG(54:5), TG(54:6), TG(56:1), TG(58:2) and TG(58:3), or a fragment, variant or derivative thereof.

15. The method of claim 14, wherein the one or more lipid biomarkers are selected from the group consisting of LPC(14:0), PE(34:1), PS(38:4), SM(d36:2), TG(52:3e) and TG(58:3), or a fragment, variant or derivative thereof.

16. The method of any one of claims 1 to 11, wherein the one or more lipid biomarkers are selected from the group consisting of LPC(14:0), LPC(16:0e), LPE(22:6), PE(34:2p), PE(38:3p), PI(36:1), PI(38:6), PS(38:4), PS(40:6), SM(d33:1), SM(d35:1) and TG(57:1), or a fragment, variant or derivative thereof.

17. The method of claim 16, wherein the one or more lipid biomarkers comprise:
(a) LPC(14:0), PS(38:4) and TG(57:1), or a fragment, variant or derivative thereof:
(b) LPC(16:0e), LPE(22:6) and PI(38:6), or a fragment, variant or derivative thereof;
(c) PE(34:2p), PE(38:3p), PI(36:1), PI(38:6) and PS(40:6), or a fragment, variant or derivative thereof:
(d) LPC(14:0), PI(38:6), and SM(d33:1), or a fragment, variant or derivative thereof: or
(e) LPC(14:0), PI(38:6), and SM(d35:1), or a fragment, variant or derivative thereof:
and optionally one or more other lipid biomarkers selected from the group consisting of LPC(14:0), LPC(16:0e), LPE(22:6), PE(34:2p), PE(38:3p), PI(36:1), PI(38:6), PS(38:4), PS(40:6), SM(d33:1), SM(d35:1) and TG(57:1), or a fragment, variant or derivative thereof.

18. The method of any one of claims 1 to 12, wherein the one or more lipid biomarkers described herein are selected from the group consisting of LPC(14:0), LPE(22:6), PI(38:6), PE(34:2p), SM(d35:1), PS(38:4) and PS(38:4), or a fragment, variant or derivative thereof.

19. The method of any one of the preceding claims, wherein the level of the one or more lipid biomarkers is or has been measured, at least in part, by mass spectrometry.

20. The method of any one of the preceding claims, wherein the predictive accuracy of the method, as determined by an ROC AUC value, is at least about 0.65, at least about 0.70, at least about 0.75 or at least about 0.80.

21. A system for determining the presence or absence of a breast cancer in a subject, the system comprising:
a mass spectrometry unit configured for determining a level of one or more lipid biomarkers in a biological sample obtained from the subject, wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof; and
a processing unit configured for using or analysing the level of the one or more lipid biomarkers to determine the presence or absence of the breast cancer in the subject.

22. A kit for determining the presence or absence of a breast cancer in a subject, the kit comprising one or more reagents for determining a level of one or more lipid biomarkers in a biological sample obtained from the subject, wherein the one or more lipid biomarkers are selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

23. The kit of claim 22, wherein the one or more reagents comprise one or more probes, each probe being specific or selective for one of the one or more lipid biomarkers.

24. The method, system or kit of any one of the preceding claims, wherein the one or more lipid biomarkers comprises two or more lipid biomarkers, three or more lipid biomarkers, four or more lipid biomarkers or five or more lipid biomarkers selected from the group consisting of Cer(d36:1), Cer(d42:0), Cer(d42:1), DG(34:2), Hex2Cer(d34:1), Hex2Cer(d42:2), LPC(14:0), LPC(16:0e), LPC(17:1), LPC(18:3), LPE(22:6), LPI(20:4), PC(32:1), PC(34:1), PC(35:4), PC(36:2), PC(36:3), PC(37:4), PC(38:5), PE(34:1), PE(34:2p), PE(36:4), PE(38:3p), PE(38:4), PE(O-38:5), PE(O-40:6), PI(32:1), PI(34:1), PI(36:1), PI(38:6), PS(38:4), PS(40:6), PS(40:7), SM(d32:2), SM(d33:1), SM(d35:1), SM(d36:1), SM(d36:2), SM(d38:4), SM(d40:3), SM(d41:2), SM(d41:3), SM(d42:1), SM(d42:4), SM(d44:4), TG(52:3e), TG(53:4), TG(54:3), TG(54:4), TG(54:5), TG(54:6), TG(56:1), TG(57:1), TG(58:1), TG(58:2), TG(58:3), TG(59:2), TG(60:1) and TG(62:2), or a fragment, variant or derivative thereof.

25. The method, system or kit of claim 24, wherein the one or more lipid biomarkers comprises two or more lipid biomarkers, three or more lipid biomarkers, four or more lipid biomarkers or five or more lipid biomarkers selected from the group consisting of LPC(14:0), PC(32:1), PC(38:5), PE(34:1), PS(38:4), SM(d36:2), SM(d38:4), TG(54:4), TG(56:1) and TG(58:2), or a fragment, variant or derivative thereof.

26. The method, system or kit of claim 24, wherein the one or more lipid biomarkers comprises two or more lipid biomarkers, three or more lipid biomarkers, four or more lipid biomarkers or five or more lipid biomarkers selected from the group consisting of LPC(14:0), PC(32:1), PC(36:2), PC(38:5), PE(34:1), PI(34:1), PS(38:4), SM(d36:2), SM(d38:4), SM(d44:4), TG(52:3e), TG(53:4), TG(54:4), TG(54:5), TG(54:6), TG(56:1), TG(58:2) and TG(58:3), or a fragment, variant or derivative thereof.

27. The method, system or kit of claim 26, wherein the one or more lipid biomarkers comprises two or more lipid biomarkers, three or more lipid biomarkers, four or more lipid biomarkers or five or more lipid biomarkers selected from the group consisting of LPC(14:0), PE(34:1), PS(38:4), SM(d36:2), TG(52:3e) and TG(58:3), or a fragment, variant or derivative thereof.

28. The method, system or kit of claim 24, wherein the one or more lipid biomarkers comprises two or more lipid biomarkers, three or more lipid biomarkers, four or more lipid biomarkers or five or more lipid biomarkers selected from the group consisting of LPC(14:0), LPC(16:0e), LPE(22:6), PE(34:2p), PE(38:3p), PI(36:1), PI(38:6), PS(38:4), PS(40:6), SM(d33:1), SM(d35:1) and TG(57:1), or a fragment, variant or derivative thereof.

29. The method, system or kit of any one of the preceding claims, wherein the biological sample is or comprises a blood sample, a plasma sample and/or a serum sample.

30. The system or kit of any one of claims 21 to 29, for use in the method of any one of 1 to 20.
